SEQUENCE LISTING (49 sequences)

SEQ ID NO: 1 - DNA, 1117 bp, Homo sapiens
tctagagctagcgaattcctttctccccgccgcattcccggtgtcgacttactagctgcaagcctctgcctgccttcctgcgcgccgttccccgctagtcgctgctgctggcgcgcactcgccgggtttttcctcccacggcctcgagatggtggtgaatgtggcacggaggagccgggccttccaacccggtgggcccgagctccgaaaggccccctcggcagtgagaggggcgggagcccgcgggggccgcgcccttctctcgcttcggactgcgcaacgctgcgctctgggctgacaggcggataaaacggtcccatcaagactgagaaaaagcacaccagctattggcacagcgtgggcagtggggcctacaggatgactgacttagtctacagagatcccggcgtacttaagcagatgaagactcttaagatgacagaaggtgatttttctggtgatcgaggacttccggggtaatgacagtgatgaaatgcaggggacctggttgcccccaagtttcctggcagtgtgtgatactgaggaggtgagcttgtttctggagctgtgctttaaggtaaagttgatcagcttaatcctcctgatccctttcccatcggatctgaacactggtcttggtggtcgtaaaaggaggaaaagtaatagtgaagctggcctaaatgttgtaatctggtatatggcatgtgggctagtttcagacaggtttcagagatggttggatctctgaaattgtaaaatgaagtataatcttaggctaagggaaggatgcgtgtgaagctctggaggttggtatagtaatagctgacctattactgcatttgggagggatctgtcatagcttccttgcctcttaattaagggtggtgtttttttcttttagattcatgttacatgtaaagctgtcctcatttgtgactatggacctatggagttgggacaatctctatgggaagcagaaggcaaggaccccggtcattttaggtagaaacaacagcatgctaatgcaaaaaattatgcagtgtgctactgaacttcagaggtgatcaataaaagaagaataaaaagactaataaaagtagaattctgatcaggatcc

SEQ ID NO: 2 - DNA, 557 bp, Homo sapiens
tcggatctgaacactggtcttggtggtcgtaaaaggaggaaaagtaatagtgaagctggcctaaatgttgtaatctggtatatggcatgtgggctagtttcagacaggtttcagagatggttggatctctgaaattgtaaaatgaagtataatcttaggctaagggaaggatgcgtgtgaagctctggaggttggtatagtaatagctgacctattactgcatttgggagggatctgtcatagcttccttgcctcttaattaagggtggtgtttttttcttttagattcatgttacatgtaaagctgtcctcatttgtgactatggacctatggagttgggacaatctctatgggaagcagaaggcaaggaccccggtcattttaggtagaaacaacagcatgctaatgcaaaaaattatgcagtgtgctactgaacttcagaggtgatcaataaaagaagaataaaaagactaataaaagtagaattctgatcaggatcctctagagctagcgaattcctttctccccgccgcattcccggtgtcgacttactagc

SEQ ID NO: 3 - DNA, 642 bp, Mus musculus
tctagagctagcgaattctttctcgcttcatccgcggtccctgtctgttttcgttatggcgccttgtacttctacccaacgttgcccgccccccatcttcccagcccaccagctccgtccgcctctccggatgattcgtgagataccgagcctgccgggaagggaccggatttttccgtctggtccaaggaagcacgggttatggccacatgcgcagtgataactcagggccccacgccttttgacccctgttgatgaagattgtgaggagggagaccagaagataatggacaacgatacagcagaagggtctggttacacttgagcttatttcttggaagagtctgagaagacggcctgtcaggctcctaacactaagggccatgtaaccagtgaagcagccattatatatgatttggtctacacttgttggaaaaattccacaagaggatctcctggaagctagcagaagtttttggcttgtacattgccaggcagccattggaatcggagctgcagtcagattaaggccagcctggcctacattgagaaacctcattttgggaaagtgaaatactttgtcaattaacatgcagttggttcaataaagacttttggaaagtggaattctgatcaggatcc

SEQ ID NO: 4 - DNA, 913 bp, Homo sapiens
ctttctccccgccgcattcccggtgtcgacttactagctgcaagcctctgcctgccttcctgcgcgccgttccccgctagtcgctgctgctggcgcgcactcgccgggtttttcctcccacggcctcgagatggtggtgaatgtggcacggaggagccgggccttccaacccggtgggcccgagctccgaaaggccccctcggcagtgagaggggcgggagcccgcgggggccgcgcccttctctcgcttcggactgcgcaacgctgcgctctgggctgacaggcggataaaacggtcccatcaagactgagaaaaagcacaccagctattggcacagcgtgggcagtggggcctacaggatgactgacttagtctacagagatcccggcgtacttaagcagatgaagactcttaagatgacagaaggtgatttttctggtgatcgaggacttccggggtaatgacagtgatgaaatgcaggggacctggttgcccccaagtttcctggcagtgtgtgatactgaggaggtgagcttgtttctggagctgtgctttaaggtaaagttgatcagcttaatcctcctgatccctttcccatcggatctgaacactggtcttggtggtcgtaaaaggaggaaaagtaatagtgaagctggcctaaatgttgtaatctggtatatggcatgtgggctagtttcagacaggtttcagagatggttggatctctgaaattgtaaaatgaagtataatcttaggctaagggaaggatgcgtgtgaagctctggaggttggtatagtaatagctgacctattactgcatttgggagggatctgtcatagcttccttgcctcttaattaagggtggtgtttttttcttttataaaagaagaataaaaagactaataaaagta

SEQ ID NO: 5 - DNA, 602 bp, Homo sapiens
ctttctccccgccgcattcccggtgtcgacttactagctgcaagcctctgcctgccttcctgcgcgccgttccccgctagtcgctgctgctggcgcgcactcgccgggtttttcctcccacggcctcgagcactggtcttggtggtcgtaaaaggaggaaaagtaatagtgaagctggcctaaatgttgtaatctggtatatggcatgtgggctagtttcagacaggtttcagagatggttggatctctgaaattgtaaaatgaagtataatcttaggctaagggaaggatgcgtgtgaagctctggaggttggtatagtaatagctgacctattactgcatttgggagggatctgtcatagcttccttgcctcttaattaagggtggtgtttttttcttttagattcatgttacatgtaaagctgtcctcatttgtgactatggacctatggagttgggacaatctctatgggaagcagaaggcaaggaccccggtcattttaggtagaaacaacagcatgctaatgcaaaaaattatgcagtgtgctactgaacttcagaggtgatcaataaaagaagaataaaaagactaataaaagta

SEQ ID NO: 6 - DNA, 969 bp, Homo sapiens
ctttctccccgccgcattcccggtgtcgacttactagctgcaagcctctgcctgccttcctgcgcgccgttccccgctagtcgctgctgctggcgcgcactcgccgggtttttcctcccacggcctcgagatggtggtgaatgtggcacggaggagccgggccttccaacccggtgggcccgagctccgaaaggccccctcggcagtgagaggggcgggagcccgcgggggccgcgcccttctctcgcttcggactgcgcaacgctgcgctctgggctgacaggcggataaaacggtcccatcaagactgagaaaaagcacaccagctattggcacagcgtgggcagtggggcctacaggatgactgacttagtctacagagatcccggcgtacttaagcagatgaagactcttaagatgacagaaggtgatttttctggtgatcgaggacttccggggtaatgacagtgatgaaatgcaggggacctggttgcccccaagtttcctggcagtgtgtgatactgaggaggtgagcttgtttctggagctgtgctttaaggtaaagttgatcagcttaatcctcctgatccctttcccatcggatctgaacactggtcttggtggtcgtaaaaggaggaaaagtggctaagggaaggatgcgtgtgaagctctggaggttggtatagtaatagctgacctattactgcatttgggagggatctgtcatagcttccttgcctcttaattaagggtggtgtttttttcttttagattcatgttacatgtaaagctgtcctcatttgtgactatggacctatggagttgggacaatctctatgggaagcagaaggcaaggaccccggtcattttaggtagaaacaacagcatgctaatgcaaaaaattatgcagtgtgctactgaacttcagaggtgatcaataaaagaagaataaaaagactaataaaagta

SEQ ID NO: 7 - DNA, 560 bp, Homo sapiens
tgcaagcctctgcctgccttcctgcgcgccgttccccgctagtcgctgctgctggcgcgcactcgccgggtttttcctcccacggcctcgagatggtggtgaatgtggcacggaggagccgggccttccaacccggtgggcccgagctccgaaaggccccctcggcagtgagaggggcgggagcccgcgggggccgcgcccttctctcgcttcggactgcgcaacgctgcgctctgggctgacaggcggataaaacggtcccatcaagactgagaaaaagcacaccagctattggcacagcgtgggcagtggggcctacaggatgactgacttagtctacagagatcccggcgtacttaagcagatgaagactcttaagatgacagaaggtgatttttctggtgatcgaggacttccggggtaatgacagtgatgaaatgcaggggacctggttgcccccaagtttcctggcagtgtgtgatactgaggaggtgagcttgtttctggagctgtgctttaaggtaaagttgatcagcttaatcctcctgatccctttccca

SEQ ID NO: 8 - DNA, 22 bp, Mus musculus
gaaatcgtgcgtgacatcaaag

SEQ ID NO: 9 - DNA, 22 bp, Mus musculus
tgtagtttcatggatgccacag

SEQ ID NO: 10 - DNA, 21 bp, Mus musculus
aggtcggtgtgaacggatttg

SEQ ID NO: 11 - DNA, 23 bp, Mus musculus
tgtagaccatgtagttgaggtca

SEQ ID NO: 12 - DNA, 20 bp, Mus musculus
tccaaggaagcacgggttat

SEQ ID NO: 13 - DNA, 20 bp, Mus musculus
ttctggtctccctcctcaca

SEQ ID NO: 14 - DNA, 19 bp, Mus musculus
cctggtgatgtccgacctg

SEQ ID NO: 15 - DNA, 19 bp, Mus musculus
ccatgagcgcatcgcaatc

SEQ ID NO: 16 - DNA, 20 bp, Mus musculus
cgcaggttcttggtcactgt

SEQ ID NO: 17 - DNA, 20 bp, Mus musculus
tgttcacgaaagccagagcg

SEQ ID NO: 18 - DNA, 22 bp, Mus musculus
tcaaacgtgagagtgtctaacg

SEQ ID NO: 19 - DNA, 20 bp, Mus musculus
ccgggccgaagagatttctg

SEQ ID NO: 20 - DNA, 26 bp, Homo sapiens
gctataaattctttgctgacctgctg

SEQ ID NO: 21 - DNA, 28 bp, Homo sapiens
aattacttttatgtcccctgttgactgg

SEQ ID NO: 22 - DNA, 20 bp, Homo sapiens
caggaatgtgggacatggaa

SEQ ID NO: 23 - DNA, 20 bp, Homo sapiens
ggtgtggagcctggatcttt

SEQ ID NO: 24 - DNA, 19 bp, Homo sapiens
tgtccgtcagaacccatgc

SEQ ID NO: 25 - DNA, 21 bp, Homo sapiens
aaagtcgaagttccatcgctc

SEQ ID NO: 26 - DNA, 18 bp, Homo sapiens
gggggcaccagaggcagt

SEQ ID NO: 27 - DNA, 19 bp, Homo sapiens
ggttgtggcgggggcagtt

SEQ ID NO: 28 - DNA, 20 bp, Homo sapiens
aacgtgcgagtgtctaacgg

SEQ ID NO: 29 - DNA, 22 bp, Homo sapiens
ccctctaggggtttgtgattct

SEQ ID NO: 30 - DNA, 23 bp, Homo sapiens
ccttgcctcttaattaagggtgg

SEQ ID NO: 31 - DNA, 21 bp, Homo sapiens
cttgccttctgcttcccatag

SEQ ID NO: 32 - DNA, 23 bp, Homo sapiens
accaccccaatggatgcagacag

SEQ ID NO: 33 - DNA, 20 bp, Homo sapiens
acgggctaagcgtctggcac

SEQ ID NO: 34 - DNA, 19 bp, Homo sapiens
cctggccggtcatcaactg

SEQ ID NO: 35 - DNA, 21 bp, Homo sapiens
agtaaggtgcgatcttctggc

SEQ ID NO: 36 - DNA, 20 bp, Homo sapiens
ggattccaggaaggagcgag

SEQ ID NO: 37 - DNA, 20 bp, Homo sapiens
attgccgacctcacggattt

SEQ ID NO: 38 - DNA, 22 bp, Homo sapiens
atagctgctgtggtcaaaaagg

SEQ ID NO: 39 - DNA, 25 bp, Homo sapiens
ttgcagatcagttacaacaaacaga

SEQ ID NO: 40 - DNA, 20 bp, Homo sapiens
aagggctgtggctggtcata

SEQ ID NO: 41 - DNA, 23 bp, Homo sapiens
gcagcttgcaggtattgtactga

SEQ ID NO: 42 - DNA, 24 bp, Homo sapiens
tgacagttttccttgacggtcgcc

SEQ ID NO: 43 - DNA, 24 bp, Homo sapiens
ccatgggatctccaactgcatgca

SEQ ID NO: 44 - DNA, 21 bp, Homo sapiens
ccagaacacaggcagctacag

SEQ ID NO: 45 - DNA, 21 bp, Homo sapiens
tcttccaatgcatcatctcca

SEQ ID NO: 46 - DNA, 20 bp, Sus Scrofa
ccccttcattgacctccact

SEQ ID NO: 47 - DNA, 20 bp, Sus Scrofa
ttgactgtgccgtggaactt

SEQ ID NO: 48 - DNA, 21 bp, Sus Scrofa
tgacctttccagtgagggaag

SEQ ID NO: 49 - DNA, 20 bp, Sus Scrofa
gctccaggaaaagctggtgt

(19) United States
(12) Patent Application Publication
(10) Pub. No.: US 2022/0010310 A1
(43) Pub. Date: Jan. 13, 2022

(54) An lncRNA integrates a DNA-PK-mediated DNA damage response and vascular senescence

(71) Applicant: The Brigham and Women`s Hospital, Inc., Boston, MA (US)

(72) Inventors: Mark W. Feinberg, Newton, MA (US); Stefan Haemmig, Brookline, MA (US)

(21) Appl. No.: 17/291,922

(22) PCT Filed: Nov. 12, 2019
(86) PCT No.: PCT/US2019/061006

§ 371 Date: May 6, 2021

Related U.S. Application Data
(60) Provisional application No. 62/757,832, filed on Nov. 9, 2018.
(60) Provisional application No. 62/905,479, filed on Sep. 25, 2019.

(51) Int. Cl. C12N 15/113; C12N 15/86

(52) U.S. Cl. CPC C12N 15/113; C12N 15/86; C12N 2310/3521; C12N 2310/321; C12N 2310/317

(57) ABSTRACT

Methods and compositions for use in treating subjects suffering from a disease associated with a malfunction in DNA repair response using compositions that comprise or encode SNHG12 long non-coding RNA.

### CLAIM OF PRIORITY

This application claims the benefit of U.S. Provisional Application Ser. Nos. 62/757,832 and 62/905,479 filed on Nov. 9, 2018 and Sep. 25, 2019, respectively. The entire contents of the foregoing are incorporated herein by reference.

### FEDERALLY SPONSORED RESEARCH OR DEVELOPMENT

This invention was made with Government support under Grant Nos. HL115141, HL117994, HL134849, GM115605, and HL134892 awarded by the National Institutes of Health. The Government has certain rights in the invention.

### TECHNICAL FIELD

Described herein are compositions comprising lncRNA SNHG12 and methods of use thereof.

### BACKGROUND

Atherosclerosis, a chronic arterial disease of medium-to-large sized arteries, is the most frequent cause of death worldwide and depends on traditional risk factors including lipoprotein accumulation, as well as immune cell functions, and extracellular matrix metabolism (1, 2). Accumulating studies demonstrate that cells of advanced plaques are more prone to senescence, a permanent cellular growth arrest often triggered by DNA damage (3-5). Reactive oxygen species (ROS)-mediated oxidative stress and DNA damage can contribute to cellular senescence and dysfunction of endothelial cells (ECs) and macrophages (6, 7) and thus to chronic disease such as atherosclerosis, neurodegenerative disorders, premature aging, senescence, among others (8, 9). Lesional DNA damage increases with the progression of atherosclerosis (10). The consequences of unrepaired or extensive DNA damage include growth arrest, cell senescence, and apoptosis, which all rise with plaque severity (11). Advanced plaques contain cells bearing senescence markers such as senescence-associated β-galactosidase (SA-βgal) activity and elevated expression of p16, p21, and p53 (12). Recently, elimination of senescent-positive cells (i.e. p16⁺) in advanced lesions from LDLR^{-/-} mice led to inhibition of lesion growth, prevention of maladaptive plaque remodeling, and a reduction in the secretion of pro-inflammatory molecules (13). Despite these important findings, major mechanistic gaps remain in the understanding of the underlying molecular signaling events that contribute to the increased DNA damage and senescence in advanced atherosclerotic lesions.

In addition to the above, atherosclerosis is a pathology that leads to myocardial infarction and stroke. For many years after its recognition, atherosclerosis was thought to involve passive lipid deposition in the vessel wall. Today we understand that atherosclerosis is a chronic inflammatory disease driven by lipids, specifically low density lipoproteins (LDL) and leukocytes. Neither atherosclerosis nor its complications adhere to a simple arithmetic of dietary lipid imbalance, but rather encompass a syndrome in which environmental and genetic inputs disrupt biological systems. In other words, lifestyle, age, hereditary factors, and co-morbidities disturb immune, digestive, endocrine, circulatory, and nervous systems, thereby altering immune function, metabolism, and many other processes, while eliciting inflammation, hypercholesterolemia, and hypertension. Atherosclerosis develops and causes myocardial infarction or stroke when many things go wrong in many different ways.

### SUMMARY

Provided herein are methods of treating a subject who has a disease associated with a malfunction in DNA repair response, the method comprising: administering to the subject a therapeutically effective dose of a pharmaceutical composition that increases expression of Small Nucleolar Host Gene-12 (SNHG12) long non-coding RNA in a cell of the subject in need thereof.

Also provided herein are pharmaceutical compositions for use in treating a subject suffering from a disease associated with a malfunction in DNA repair response, comprising a nucleic acid molecule comprising (i) all or part of the SNHG12 long-coding RNA sequence, or (ii) a sequence encoding all or part of the SNHG12 long-coding RNA, optionally in an expression vector

In some embodiments, the pharmaceutical composition comprises a nucleic acid molecule comprising (i) all or part of the SNHG12 long-coding RNA sequence, or (ii) a sequence, optionally in an expression vector, encoding all or part of the SNHG12 long-coding RNA.

In some embodiments, the expression vector comprises an adeno-associated virus (AAV), adenovirus, lentivirus, or a DNA plasmid.

In some embodiments, the nucleic acid molecule is an RNA molecule comprising all or part of SEQ ID NO: 1 or 2.

In some embodiments, the nucleic acid molecule comprises SEQ ID NO: 1 or 2.

In some embodiments, the nucleic acid molecule has at least 90% of sequence identity with SEQ ID NO: 1 or 2.

In some embodiments, the nucleic acid has at least 80% sequence identity with SEQ ID NO: 1 or 2 and is capable of increasing the expression of Small Nucleolar Host Gene-12 (SNHG12) long non-coding RNA.

In some embodiments, wherein the composition is administered to the subject parenterally, intramuscularly, intravitreally, subcutaneously, arterially, intravenously, topically, orally, or by local administration, such as by aerosol or transdermally.

In some embodiments, the nucleic acid molecule comprises a chemical modification that improves one or more, or all, of nuclease stability, decreased likelihood of triggering an innate immune response, lowering incidence of off-target effects, and improved pharmacodynamics relative to a non-modified nucleic acid.

In some embodiments, the at least one chemical modification comprises a modification selected from phosphorothioate, boranophosphate, 4′-thio-ribose, locked nucleic acid, 2′-O-(2-methoxyethyl), 2′-O-alkyl, 2′-O-alkyl-O-alkyl, 2′-O-methyl, 2′-fluoro, 2′-amino, or 2′-deoxy-2′-fluoro-b-D-arabinonucleic acid.

In some embodiments, the at least one chemical modification comprises a 5′ cap.

In some embodiments, the composition is administered to the tunica intima of the subject.

In some embodiments, the disease is atherosclerosis, heart failure, diabetes, hypertension, neurodegenerative disease, autoimmune disease, ataxia telangiectasia, aging, Bloom's Syndrome, immunodeficiency, Cockayne syndrome, Nijmegen breakage syndrome, Trichothiodystrophy, Fanconi Anaemia, Werner Syndrome, Li-Fraumeni syndrome, xeroderma pigmentosum, senescence, Hutchinson-Gilford progeria syndrome, or cancer.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-M. Identification of the conserved lncRNA SNHG12 in lesional intima. (A) RNA derived from aortic intima of LDLR^{-/-} mice (n=3; each sample represents RNA pooled from two mice) that were placed on an HCD for 0 weeks (group 1), 2 weeks (group 2), 12 weeks (group 3), and 18 weeks after 6 weeks of resumption of a normal chow diet (group 4). (B) Workflow of genome-wide RNA-Seq profiling for the identification of differentially expressed lncRNAs (log 2-fold change (1.5); FDR<0.05). (C) Venn diagram display of dysregulated lncRNAs using DESeq2/NOR showed intersecting hits (n=14), uniquely identified in DESeq2 (n=23) or NOR (n=5). (D) 5′RACE-PCR for SNHG12 in mouse from RNA of the aortic intima and human RNA from HUVECs (n=3). IGV-based visualization of RNA-Seq for mouse and pig verified sequence alignment and splicing junctions. (E) RNA-Seq results for SNHG12 across group 1-4 were verified by RT-qPCR for SNHG12-205 isoform. P value was determined by Student's t test. (F) LDLR^{-/-} mice were i.v. injected with vehicle control-gapmeR or SNHG12-gapmeR (7.5 mg/kg/mouse) twice per week and placed on HCD for 12 weeks (n=12 per group) and (G) knockdown of SNHG12 was assessed in RNA derived from aortic intima, media, and PBMC (n=6 per group). P value was determined by Student's t test. (H) Lesion areas were quantified by ORO-stained aortic sinus sections (n=10 per group), and (I) thoracoabdominal aorta (n=12 per group). P value was determined by Student's t test. (J) ApoE^{-/-} mice were i.v. injected with LacZ or SNHG12 RNA twice per week and placed on a HCD for 6 weeks (n=10 per group). (K) Delivery of RNA to the aortic intima, tunica media, and PBMC was assessed by RT-qPCR (n=5 per group). Lesion areas were quantified by ORO staining of aortic sinus (L) and thoracoabdominal aorta (M) (n=10 per group). P value was determined by Student's t test. For all panels, values are mean±SD; *p<0.05, **p<0.01; ***p<0.001; ****p<0.0001.

FIGS. 2A-I. LncRNA SNHG12 interacts with DNA-dependent protein kinase (DNA-PK). (A) Proteins identified in pulldowns of biotinylated SNHG12; DNA-PK bound specifically to SNHG12 compared to negative control LacZ (n=2, two technical replicates each). (B) Immunoblotting for DNA-PK, ATM, ATR, Ku80, Ku70, p53, and control on nuclear protein lysate from HUVECs after lncRNA pulldown (n=5). (C) Analysis after streptavidin pulldown of nuclear protein lysate derived from the aorta of C57B1/6 mice following two i.v. injections of biotin-labeled SNHG12 compared to LacZ (n=4 per group). (D, E) Predicted secondary structure of SNHG12 with indicated deletions of domains 1˜4 used for subsequent lncRNA pulldown of biotinylated SNHG12 domain deletion constructs (n=4). P value was determined by one-way analysis of variance (ANOVA) (Fisher's test). (F) IP for DNA-PK following RNA isolation and subsequent RT-qPCR for SNHG12 and for negative control HPRT (n=5). (G) HUVEC nuclear protein lysate was harvested 36 hrs post-transfection with control-gapmeR or SNHG12-gapmeR (25 nM) in the presence or absence of H₂O₂ for 1 hr (1 mM). Subsequent IP with IgG or DNA-PK antibody. Immunoblotting for Ku70, Ku80 and DNA-PK and (H) quantification under H₂O₂ conditions (n=3). (I) Schematic depiction of SNHG12 binding properties to DNA-PK, and downstream binding of DNA-PK to Ku heterodimer. For all panels, values are mean±SD; *p<0.05, **p<0.01; ***p<0.001; ****p<0.0001.

FIGS. 3A-I. DNA damage analysis in vitro and in lesions upon SNHG12 loss- and gain-of-function studies. (A) SNHG12 expression was analyzed from HUVECs that were γ-irradiated for 1 min (1.2Gy^{-min}) and RNA was isolated at 0, 1, 2, 4, 8 and 12 hrs post-irradiation or in the absence or presence of H₂O₂ (30, 100, 250, 500 and 1000 μM) for 4 hrs. P value was determined by one-way analysis of variance (ANOVA) (Fisher's test). (B) Control-gapmeR or SNHG12-gapmeR transfected HUVECs were γ-irradiated (1.2Gy^{-min}) and fixed at 0, 1, 2, 4, 8 and 12 hrs post-irradiation. P value was determined by Student's t test. (C) Analysis of protein lysate for γH2AX from HUVECs transfected with gapmeRs or negative control treated for 0 and 30 min with H₂O₂ (1 mM) (n=3). P value was determined by Student's t test. (D) HUVECs transduced with control-lenti or SNHG12-lenti were analyzed for γH2AX in the absence or presence of H₂O₂ (1 mM) for 1 hr. P value was determined by Student's t test. (E) Lesional DNA damage in the vessel wall of the aortic arch was quantified in LDLR^{-/-} mice injected with control-gapmeR or SNHG12-gapmeR on HCD for 12 weeks by γH2AX with nuclear co-localization of DAPI in lesional CD31⁺ cells. (n=6 mice per group; 2-3 lesion per arch). P value was determined by Student's t test. (F) Lesional DNA damage was quantified in the aortic arch of ApoE^{-/-} mice after delivery of lacZ or SNHG12 RNA. (n=10 mice per group). HUVECs were co-transfected with either control-gapmeR, SNHG12-gapmeR, control-siRNA, or siRNA-DNA-PK (25 nM each) and treated with H₂O₂ (1 mM) for 1 hr before DNA double strand breaks (DSB) were assessed by (G) γH2AX Western blot (n=3) and (H) comet assay (neutral pH) (n=3). (I) MET efficiency in HUVECs was assessed by FACS for GFP positive cells, as an indicator or repaired DSB. HUVECs overexpressing SNHG12 (cherry reporter) showed more efficient NHEJ as indicated by increased number of GFP positive cells compared to lenti-control (n=3). P value was determined by Student's t test. For all panels, values are mean±SD; *p<0.05, **p<0.01; ***p<0.001; ****p<0.0001.

FIGS. 4A-K. Downstream consequences of SNHG12 on p53 and senescence. (A) Unbiased genome-wide RNA-Seq profiling of HUVECs transfected with control-gapmeR or SNHG12-gapmeR treated for 1 hr with H₂O₂ (1 mM) (n=3 per group). Volcano plot displaying significantly dysregulated genes (log 2-fold change (1.5); FDR<0.05). (B) GSEA of top 10 significantly affected processes. (C) Enrichment plot for p53 pathway. (D) ROS-induced DNA damage in gapmeR transfected HUVECs to assess p-p53 and total p53 by Western blot. P value was determined by Student's t test. (E) Electrophoretic mobility shift assay for p53 using nuclear lysate of control- or SNHG12-gapmeR transfected HUVECs. (F) Cells were treated for 1 hr with H₂O₂ (30 μM), followed by 3 days incubation in normal growth medium and analysis for p16 and p21 expression by immunoblot. (G) RT-qPCR analysis for markers of senescence from aortic endothelium derived RNA (n=6 per group). (H) Plaque necrosis was measured in the aortic sinus as indicated (n=6 per group; 2-3 lesion per mice). (I) SAβ-gal staining of lentiviral transduced HUVECs for SNHG12 as described previously under (F) (n=3). (J) Transcytosis for DiI-labeled LDL was quantified using TIRF microscopy (n=3). (K) In vivo efferocytosis measured by Mac2-associated TUNEL staining in lesions of SNHG12-gapmeR injected LDLR^{-/-} mice. P value was determined by Student's t test. For all panels, values are mean±SD; *p<0.05, **p<0.01; ***p<0.001; ****p<0.0001.

FIGS. 5A-G. NR rescued progression of atherosclerotic lesions in LDLR^{-/-} mice induced by SNHG12 silencing. (A) LDLR^{-/-} mice were i.v. injected with vehicle control or SNHG12-gapmeR (7.5 mg/kg/mouse) twice per week and placed on an HCD containing NR (400 mg/kg/day) for 12 weeks (n=12 per group). (B) Lesion areas were quantified using ORO area on aortic sinus sections (n=10 per group). Fold change was calculated compared to groups from study 1 as described in FIG. 1. P value was determined by Student's t test. (C) Lesional DNA damage in the vascular endothelium of the aortic arch was quantified in LDLR^{-/-} mice by γH2AX with nuclear co-localization with DAPI in CD31⁺ cells compared to study 1. RT-qPCR analysis for (D) SNHG12 and (E) markers of senescence p16, p21, and p27 from RNA isolated of the aortic intima of mice placed on HCD containing NR (n=6 per group). P value was determined by Student's t test. (F) Acellular areas were measured in the aortic sinus for each of the indicated groups (n=6 per group; 2-3 lesion per mice). (G) TUNEL staining was quantified in the aortic sinus for each of the indicated groups (n=7 per group). P value was determined by Student's t test. For all panels, values are mean±SD; *p<0.05, **p<0.01; ***p<0.001; ****p<0.0001.

FIGS. 6A-F. SNHG12 expression is inversely correlated with DNA damage and senescent markers in human and pig atherosclerotic specimens. (A) SNHG12 expression was analyzed from RNA isolated from non-diseased, control carotid arteries (n=8) or atherosclerotic carotid arteries (n=23). P value was determined by Student's t test. (B) Total protein was analyzed for γH2AX, p16, and p21 assessed from the same sample cohort as described in (A) normalized to GAPDH. The graphs indicate fold change relative to control arteries. P value was determined by Student's t test. (C) Fresh frozen carotid arteries cross sections from Yorkshire pigs that were placed on an HCD for up to 60 weeks were stained for ORO to assess degree of atherosclerosis progression. (D) RNA from those specimens were analyzed for SNHG12 expression by RT-qPCR normalized to GAPDH. P value was determined by one-way analysis of variance (ANOVA) (Fisher's test). (E) RNA-Seq transcriptomic analysis is shown for expression of p16 and p21. P value was determined by one-way analysis of variance (ANOVA) (Fisher's test). (F) Depiction for proposed mechanism of SNHG12 regulating atherosclerosis through a DNA-PK mediated DDR in the vascular endothelium. For all panels, values are mean±SD; *p<0.05, **p<0.01; ***p<0.001; ****p<0.0001.

FIGS. 7A-P. Identification and characterization of lncRNA SNHG12 in mouse and human cells. (A) Quantification for Mac3 staining in the aortic sinus across groups 1˜4 (n=3 per group). (B) Heatmap for cell type markers representing endothelial cells, monocytes, or vascular smooth muscle cells from RNA-Seq analyses across groups 1-4. VCAM1, VWF, TIE2 were at low end, while the rest were close to 500. (C) LncRNA candidates identified with progression and regression of atherosclerosis in the aortic intima of LDLR−/− mice. (D) RNA-ISH for SNHG12 and negative control on regions of the aorta with and without atherosclerotic lesions of LDLR−/− mice. Bar 50 μM. (E) Schematic overview of human SNHG12 exon/intron junctions and cryptically encoded small RNAs and antisense TRNAU1AP. (F) Knockdown efficiency of gapmeR-SNHG12 (25 nM) in HUVECs (n=5 per group). P value was determined by Student's t test. (G) Silencing of SNHG12 does not affect expression of cryptically encoded small RNAs (snora44, snora16a) or antisense TRNAU1AP (n=5). P value was determined by Student's t test. (H) RT-qPCR expression analysis for SNHG12 in aortic endothelium, aortic media, PBMC, and BMDM of C57B1/6 mice on chow diet (n=4 per group). P value was determined by one-way analysis of variance (ANOVA) (Fisher's test). (I) SNHG12 expression across different human tissues using GTEx portal. (J) SNHG12-gapmeR silencing reduces nuclear SNHG12 expression normalized to cytoplasmic fraction (n=3). (K) RT-qPCR analysis for RNA derived from HUVECs, (L) bEnd3 and human primary macrophages separated into cytoplasmic, nuclear, and chromatin fractions and normalized to the cytoplasmic fraction (n=3). P value was determined by Student's t test. (M) RNA-in situ hybridization for negative control- and SNHG12-probes on PFA-fixed HUVECs. (N) Coding potential assessing tool (CPAT) predicts for human and mouse SNHG12 very low coding potentials. (O) SNHG12 sequences were cloned upstream of 3×Flag-Tag cassette, transfected in 293T cells, and immunoblotted for Flag antibody. Positive control was provided with the kit. (P) RNA from HUVECs was isolated for polyA+ and polyA− enriched RNA and analyzed by RT-qPCR (n=3 per group). For all panels, values are mean±SD; *p<0.05, **p<0.01; ***p<0.001; ****p<0.0001.

FIGS. 8A-M. SNHG12 does not affect lipid metabolism or inflammation. (A-B) Mice were i.v. injected with control- or SNHG12-gapmeRs for 1, 1.87, 3.75, and 7.5 mg/kg on two constitutive days and RNA was isolated from aortic intima (A) and aortic media (B) 3 days after the last injection (n=3 per group). P value was determined by one-way analysis of variance (ANOVA) (Fisher's test). (C) Delivery of FAM-labeled gapmeR and (D) Cy5-labeled RNA to the lesions of LDLR−/− mice. Quantification shown for Mac3+ macrophages, CD4+ T cells, CD8+ T cells, and α-actin-positive vascular smooth muscle cells (αSMC) in aortic sinus sections for (E) loss- and (F) gain-of-function in vivo studies of SNHG12 (n=10 per group). (G) FACS of F4/80+, Cd11b+ monocytes for Ly6C expression isolated from PBMC of i.v. injected mice with gapmeRs or (H) SNHG12 RNA. (I, J) Serum lipid profiles were not different from (I) control- or SNHG12-gapmeR or (J) SNHG12 RNA compared to lacZ injected mice after 12 weeks (I) or 6 weeks (J) on high cholesterol diet (HCD), respectively (n=10 per group). (K) Aortic arch was stained for p65 (Alexa555), CD31 (Alexa488), and DAPI (blue) for quantification of nuclear p65+ ECs; bar graph shows quantification of p65 in CD31 cells or (L) in macrophages using Mac2 (Alexa488) (n=5 mice per group; 2-3 lesions per mouse)(M) Nuclear and cytoplasmic fractionation for assessing p65 translocation in HUVECs stimulated for 0, 5, 30 and 60 min with H₂O₂ (1 mM) normalized to USF-2 (nuclear) and GAPDH (cytoplasmic). P value was determined by Student's t test. For all panels, values are mean±SD; *p<0.05, **p<0.01; ***p<0.001; ****p<0.0001.

FIGS. 9A-I. Identification of DNA-PK as an SNHG12 interactor. (A) Illustration of lncRNA pulldown using nuclear protein extract from HUVECs for in vitro transcribed biotin-labeled SNHG12 or negative control LacZ followed by streptavidin beads pulldown. Protein eluate was sent for MS. (B) Protein hits for SNHG12 (n=2, two technical replicates each). (C) LncRNA pulldown of biotin-labeled SNHG12 compared to negative controls LacZ and antisense transcript for SNHG12 (n=3). (D) RT-qPCR analysis for intravenous delivery of biotinylated SNHG12 to the aorta (n=4 per group). P value was determined by Student's t test. (E) Microscale thermophoresis (MST) assay was performed with red-fluorescent labeled recombinant DNA-PK and T7 transcribed RNA for SNHG12 or antisense SNHG12 transcript (n=3). (F) RT-qPCR analysis of DNA-PK in HUVECs transfected for 36 hrs with control-gapmeR or SNHG12-gapmeR (25 nM) (n=3). (G) Immunoblotting for pDNA-PK(52056), pATM (S1981) and pATR(Thr1989). P value was determined by Student's t test. (H) Purified DNA-PK protein was incubated with in vitro transcribed LacZ or SNHG12 transcript (10 pmol) together with ATP (150 μM) and luminescence was measured for detecting conversion to ADP. LacZ or SNHG12 transcripts were treated with or without RNaseA (20 Units) for 1 hr at 37° C. P value was determined by Student's t test. (I) DNA-PK protein was incubated for 1 hr with the DNA-PK kinase inhibitor NU7441 (1, 10, 100 μM) to assess DNA-PK kinase activity by luminescence (n=1). For all panels, values are mean±SD; *p <0.05, **p<0.01; ***p<0.001; ****p<0.0001.

FIGS. 10A-M. SNHG12 silencing impaired DDR in ECs and macrophages. (A) Isolation of RNA from the lesser curvature (LC) and greater curvature (GC) of C57B1/6 mice followed by RTqPCR analysis (n=6, each sample represents RNA pooled from three mice). P value was determined by Student's t test. (B) RT-qPCR analysis of the aortic intima from aged C57B1/6 mice (8, 40 and 80 weeks old) (n=6 per group). P value was determined by one-way analysis of variance (ANOVA) (Fisher's test). (C) Knockdown efficiency of antisense oligonucleotides (ASO) targeting SNHG12 in mouse endothelial cells (b.End.3) and (D) their phenotypic consequences on the DDR upon 1 hr H₂O₂ (500 μM) exposure (n=3). P value was determined by one-way analysis of variance (ANOVA) (Fisher's test). (E) Knockdown efficiency of SNHG12-gapmeR transfected human arterial cells (HAECs). P value was determined by Student's t test. (F) γH2AX immunoblot of SNHG12-gapmeR transfected HAECs treated for 1 hr with H2O2 500 μM) (n=3). (G) HUVECs overexpressing SNHG12 or control were γ-irradiated (1.2 Gy^{-min}) and PFA fixed at 0, 1, 2, 4, 8 and 12 hrs post-irradiation. (H) SNHG12 expression was assessed in HUVECs transduced with lenti-control or lenti-SNHG12 after 3 days (n=3). (I) Transfection of control-gapmeR or SNHG12-gapmeR (25 nM) in RAW264.7 cells (left) or human primary macrophages (right) showing reduced expression for SNHG12 by RT-qPCR (n=3). (J) GapmeR-transfected human primary macrophages were cultured for 2 hrs in the presence with camptothecin (CPT) (100 μM). Tail-length was quantified using pH neutral comet assay and (K) by Western blot for γH2AX. (L) Lesional DNA damage in the vessel wall of the aortic arch was quantified in LDLR−/− mice injected with control-gapmeR or SNHG12-gapmeR on HCD for 12 weeks by γH2AX with nuclear co-localization f DAPI in lesional Mac2+ macrophages. (n=6 mice per group; 2-3 lesion per arch). P value was determined by Student's t test. (M) FACS gating for NHEJ efficiency in HUVECs for GFP positive cells. P value was determined by Student's t test. For all panels, values are mean±SD; *p<0.05, **p<0.01; ***p<0.001; ****p<0.0001.

FIGS. 11A-M. Phenotypic effects of SNHG12 on senescence, EC permeability, and efferocytosis. (A) Quantification of plaque necrosis in ApoE−/− mice injected with SNHG12 RNA over the course of 6 weeks on HCD (n=10 per group). (B) Quantification of SA-βgal staining 4 days after gapmeR-transfected HUVECs were treated for 1 hr with H₂O₂ (30 μM). Representative image. Bar, 300 μM (n=3). (C) SNHG12 expression was quantified by RT-qPCR to assess knockdown efficiency from HUVECs as described in (B). (D,E) SA-βgal staining (D) and SNHG12 knockdown verification (E) in HAECs as described in (B,C) (n=3). (F) Transcytosis in ECs transfected with lenti-control or lenti-SNHG12 transduced cells in the absence or presence of H2O2 (250 μM) for 1 hr in HCAECs (n=3). (G-I) A constrictive cuff (CC) was placed on the left common carotid artery (LCCA) and Evans Blue extravasation was measured downstream of the CC 24 hrs post-surgery in C56B1/6 mice (n=9 per group) following two constitutive i.v. injections of the indicated (H) gapmeRs (7.5 mg/kg) or (I) RNA (15 μg). (J) RT-qPCR of SNHG12 expression was quantified to verify knockdown or delivery as described in (H,I) (K) GapmeR-transfected or (L) lentiviral transduced primary human macrophages were incubated with Calcein AM-labeled apoptotic Jurkat cells for 1 hr at a 10:1 ratio. Efferocytosis was quantified as total percentage of macrophages that were Calcein+ (<3 μm). (M) RT-qPCR for SNHG12 expression was performed from HUVECs transduced with lentivirus for SNHG12 used for efferocytosis analysis (n=3 per group). P value was determined by Student's t test. For all panels, values are mean±SD; *p<0.05, **p<0.01; ***p<0.001; ****p<0.0001.

FIGS. 12A-G SNHG12 has no regulatory role on apoptosis. (A-B) Apoptosis was assessed by measuring luminescence for cleaved caspase-3 and normalized to cell viability for (A) loss- and (B) gain-of-function experiments in the absence or presence of 1 hr H₂O₂ (1 mM) (n=3). (C) Quantification of cleaved-Caspase-3 staining in lesions of the aortic arch is shown (n=5 mice per group; 2-3 lesions per mouse). (D,E) TUNEL+ cells were quantified in lesions of the aortic sinus for vehicle control and SNHG12-gapmeR injected LDLR−/− mice (D) or in lesions of the aortic sinus after delivery of RNA SNHG12 or control injected ApoE−/− mice (E) (n=10-11 per group). (F,G) Incorporation of BrdU in HUVECs after (F) knockdown or (G) overexpressing SNHG12 compared to negative control in the absence or presence of 1 hr H2O2 (1 mM). P value was determined by Student's t test. For all panels, values are mean±SD; *p<0.05, **p<0.01; ***p<0.001; ****p<0.0001.

FIGS. 13A-D. Accumulating DNA damage and its effect on mitochondrial stress. (A) Oxidative consumption rate (OCR) and extracellular acidification rate (ECAR). The bars from left to right represent control, control+NR, SNHG12 KD, and SNHG12 KD+NR, respectively. (B) in HUVECs transfected with the indicated gapmeRs in the absence or presence of NR (500 μM, 24 hrs) (n=3). P value was determined by one-way analysis of variance (ANOVA) (Fisher's test). The bars from left to right represent control, control+NR, SNHG12 KD, and SNHG12 KD+NR, respectively. (C) SNHG12 expression or (D) ratio of NAD+ to NADPH in gapmeR-transfected HUVECs in the absence or presence of NR (n=3). P value was determined by Student's t test. For all panels, values are mean±SD; *p<0.05, **p<0.01; ***p<0.001; ****p<0.0001.

### DETAILED DESCRIPTION

Long noncoding RNAs (lncRNAs) are defined as RNA molecules greater than 200 nucleotides in length that have low protein-coding potential. Traditionally viewed as transcriptional noise, they are now emerging as important regulators of cellular functions such as protein synthesis, RNA maturation/transport, chromatin remodeling, and transcriptional activation and/or repression programs because of their ability to interact with RNA, DNA, or protein depending in part on their cellular localization (14). They have been shown to influence biological processes such as stem cell pluripotency, cell cycle, and DNA damage response. While lncRNAs have a low cross-species conservation rate and may have lower copy numbers per cell than mRNAs (15, 16), several studies have identified lncRNAs enriched in a tissue- or cell-specific manner that can exert profound phenotypic effects (17). Identification of lncRNAs specifically expressed in the intima of lesions during the progression phase of atherosclerosis may provide a better understanding for their roles in a stage-specific manner and potentially uncover new insights for repairing or reducing DNA damage in advanced lesions.

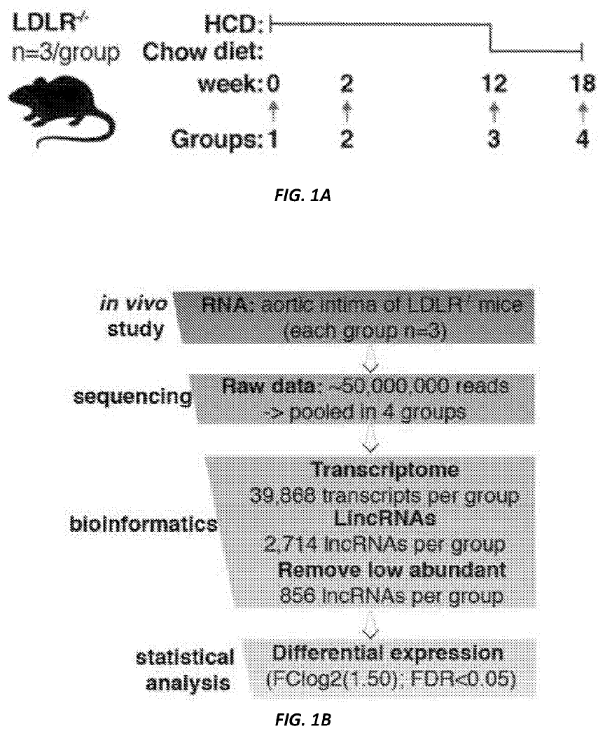

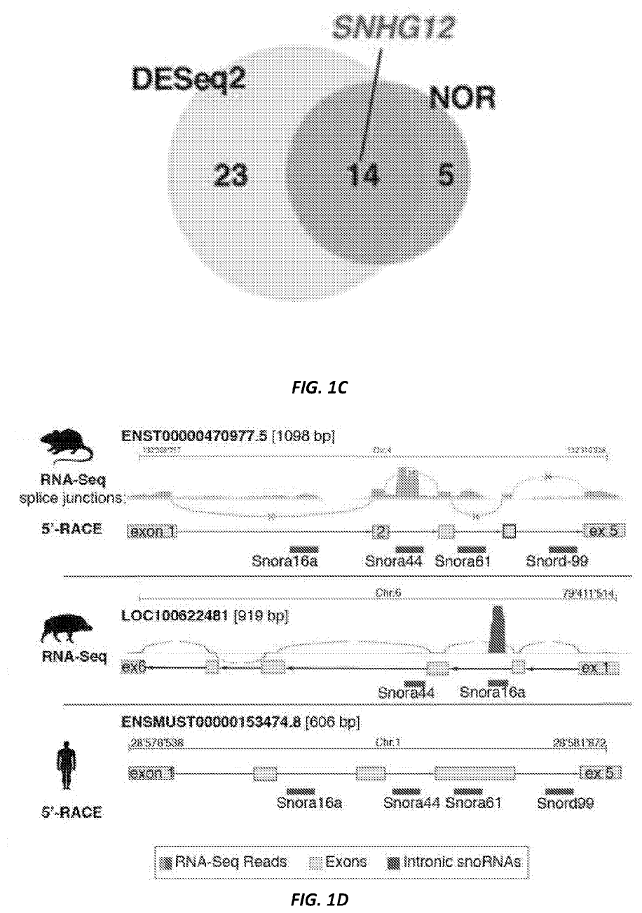

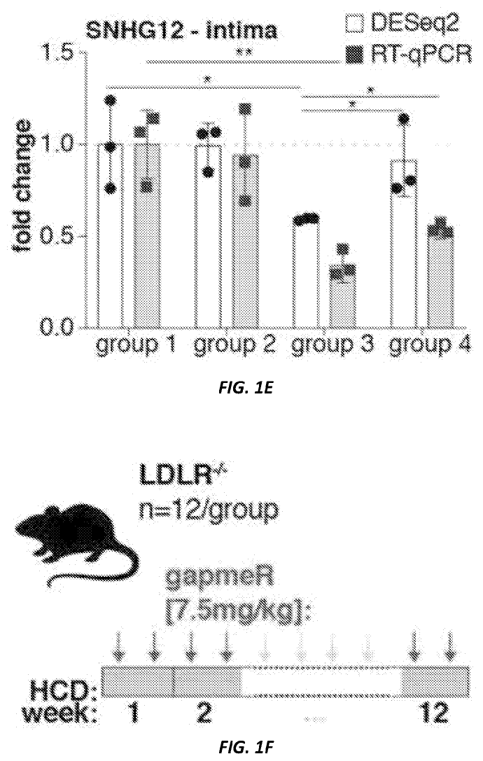

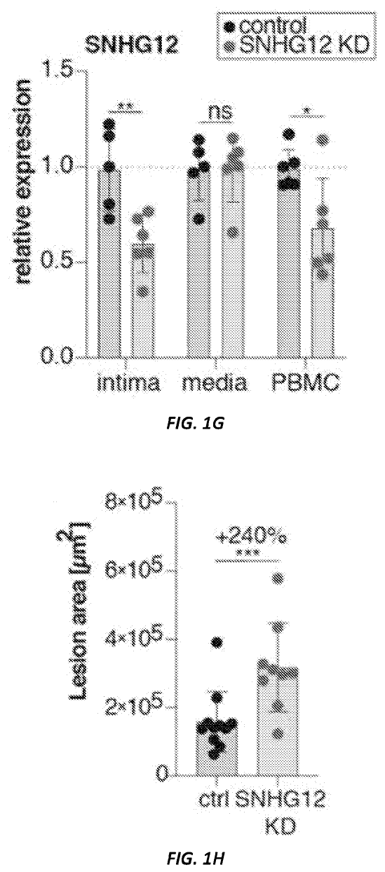

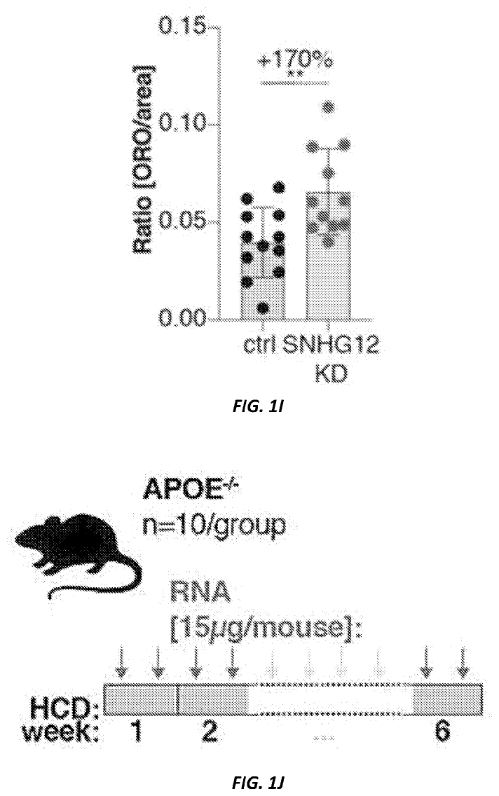

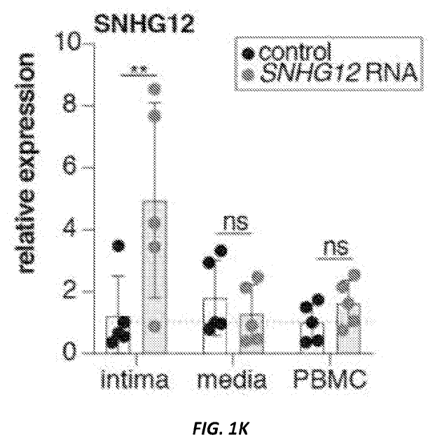

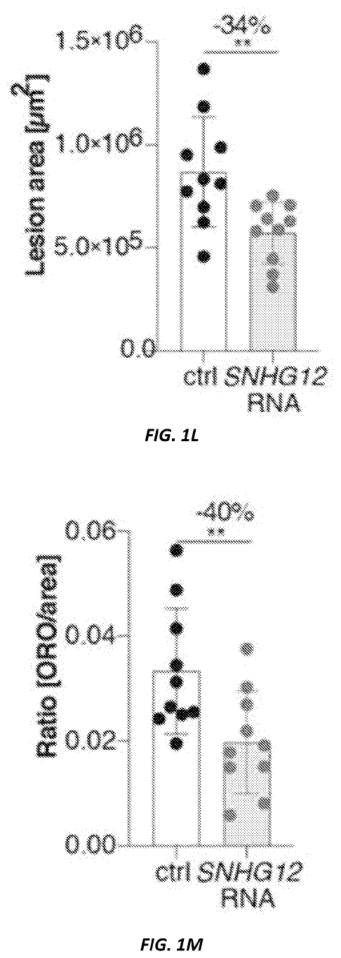

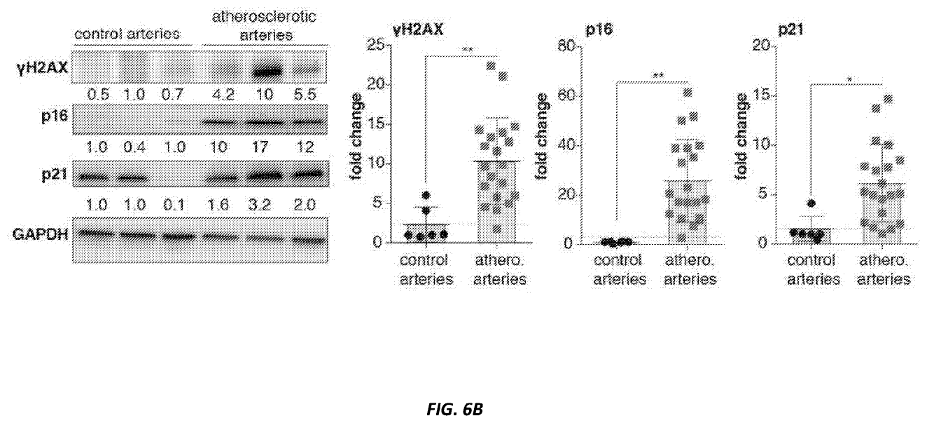

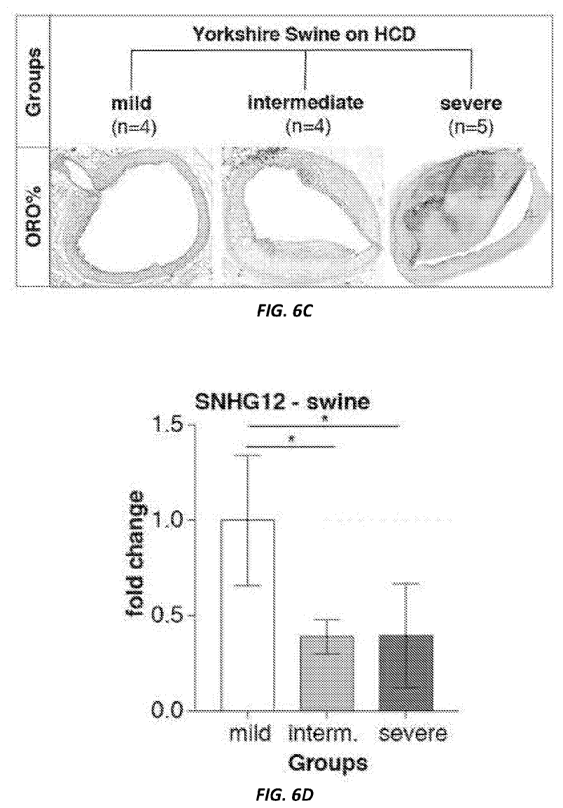

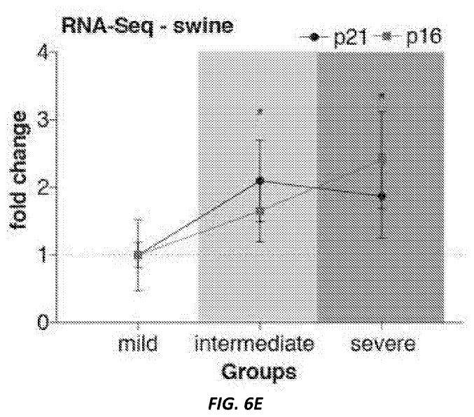

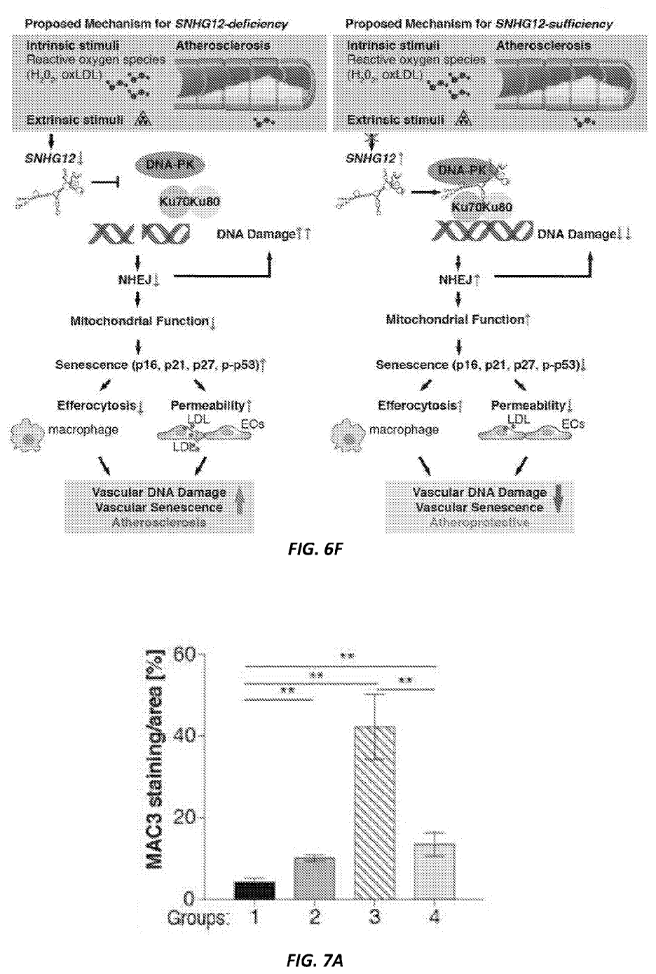

The Examples provide evidence for dynamic regulation of lncRNA SNHG12 towards stress-induced DNA damage. In support, lncRNA SNHG12 expression fell in intimal lesions during the progression phase after 12 weeks of HCD, whereas SNHG12 expression nearly normalized during the regression phase after resumption of 6 weeks of normal chow diet. Consistent with findings in mice, the expression of this evolutionary conserved lncRNA fell markedly in atherosclerotic arteries of pigs and humans, and correlated inversely with DNA damage and markers for senescence (FIG. 6). In vivo knockdown of SNHG12 in the tunica intima recapitulated this finding—reduced SNHG12 expression accelerated the progression of atherosclerosis in LDLR−/− mice. Conversely, delivery (i.v.) of SNHG12 RNA to the tunica intima, reduced plaque burden in atherosclerotic-prone mice. The studies herein demonstrated successful delivery of a lncRNA to the vessel wall showing an alteration in cardiovascular disease, such as atherosclerosis (FIG. 1). This modulation in atherosclerosis occurred without altered lipoprotein profile, lesional accumulation of leukocyte subsets, or intimal NF-kappaB activation, indicating that SNHG12 operates in a manner distinct from lipid risk factors or inflammation. Further interrogation of SNHG12-deficient lesions revealed marked increases in DNA damage in intimal ECs and macrophages as well as senescence markers. However, apoptosis did not change in lesional or cultured ECs upon loss- or gain-of-function of SNHG12.

Mechanistically, SNHG12 knockdown increased markers of DSBs (e.g. gammaH2AX) in part by inhibiting the DNA-PK interaction with Ku70 and Ku80, heterodimeric proteins that bind DSBs and that facilitate the NHJ pathway. These findings identify SNHG12 as a regulator of DNA-PK and the DDR in vitro and in vivo. While SNHG12 can increase DNA-PKcs activity, SNHG12 is probably not essential for V(D)J recombination function of DNA-PK as other studies have shown that minimal DNA-PKcs protein is suffice to mediate V(D)J recombination, but not the DDR evoked by ionizing radiation (33).

Sustained DNA damage may lead to cellular senescence and aggravate the pathogenesis of chronic disease states such as atherosclerosis. While a definitive role for DNA-PK in atherosclerotic lesion progression is poorly defined, DNA-PK activity increases with progression of atherosclerosis potentially as a means to repair DNA damage observed in the vessel wall (34). Furthermore, markers of DNA DSBs, oxidative DNA damage, and DNA reparative enzymes increase with advanced atherosclerotic lesions across mice, rabbits, and humans, highlighting the evolutionary conservation of this pathway, an indication of its potential importance (34-37). In addition to atherosclerosis, the maintenance of genomic integrity resists malignant transformation of a cell provoked by genotoxic stress and carcinogenic insults such as irradiation (38). To date, SNHG12 has been described in several forms of cancer as for example in prostate (39), gastric (40) and breast cancer (41). Upregulation of SNHG12 in some cancer cell types increased cellular proliferation and resistance to cell death insults in vitro (42). However, a definitive in vivo role of SNHG12 in Murine tumors is lacking. The findings from this study may have translational value not only for atherosclerosis, but also for cancer as this study demonstrates the regulatory role of the lncRNA SNGH12 in maintenance of cellular genomic stability by interacting with DNA-PK.

While SNHG12 knockdown elevated markers for DSBs and senescence primarily in the vascular endothelium and macrophages of lesions, we cannot rule out the possibility of similar effects in other cell types such as vascular smooth muscle cells (VSMCs). This possibility arises as delivery of ASO SNGH12 by intravenous tail vein injection may not penetrate the VSMC-enriched aortic media sufficiently to reduce SNHG12 expression. The recent recognition that VSMC-derived DNA damage had minimal effects on atherogenesis, but altered fibrous cap areas in advanced lesions, also suggests a modest contribution of DNA damage derived from this cell type (43).

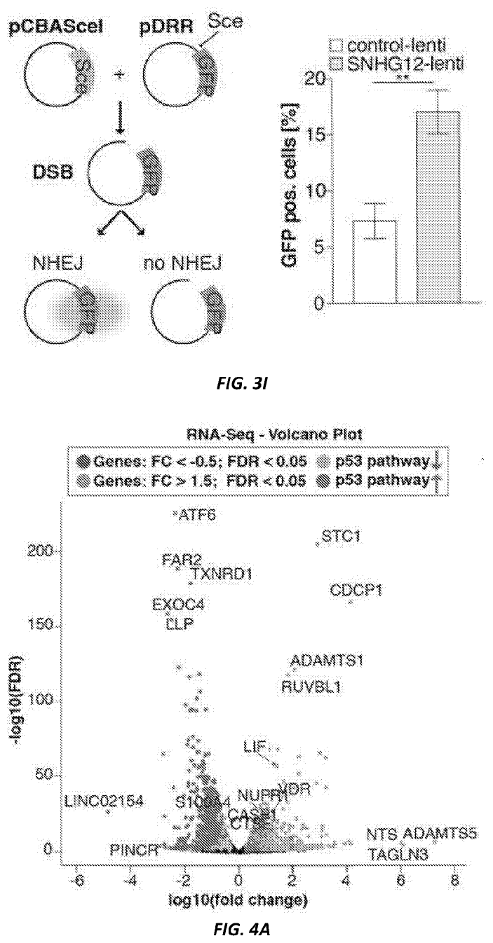

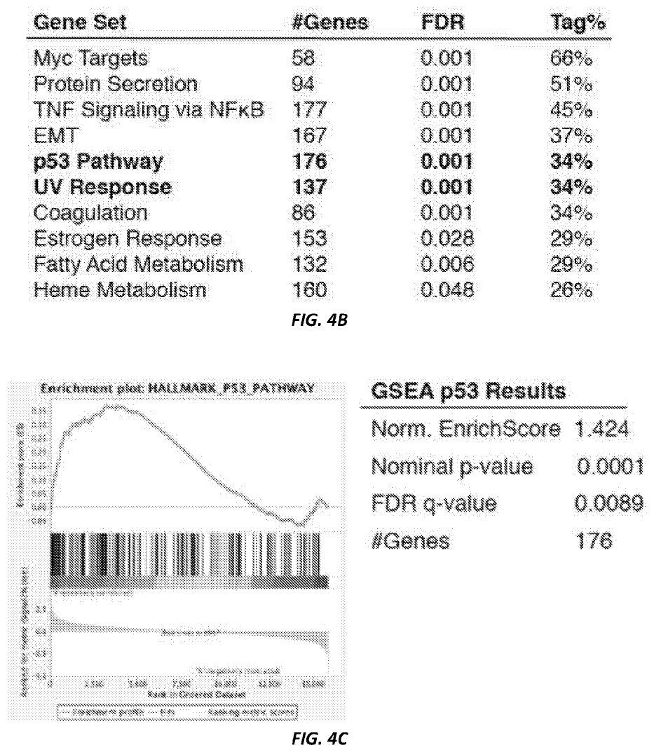

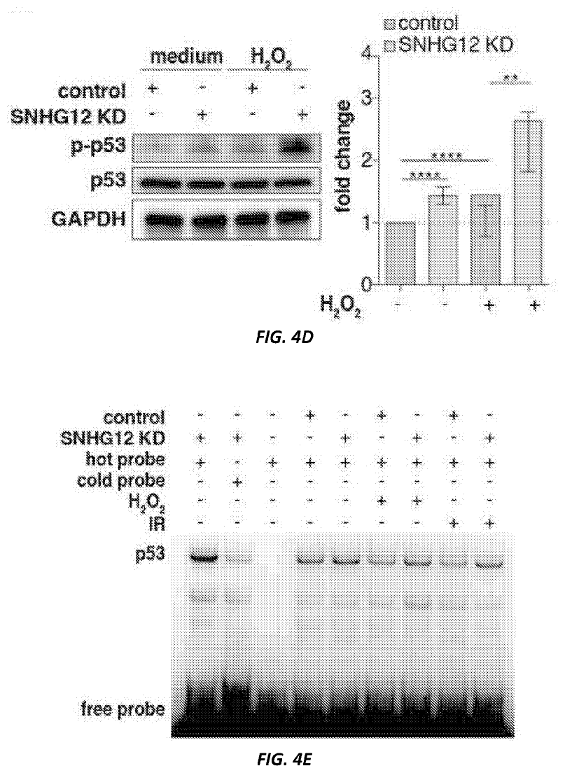

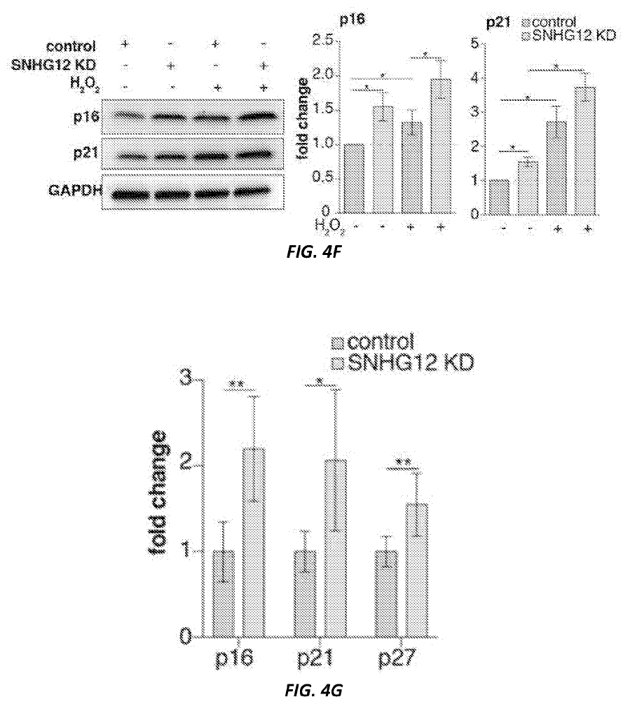

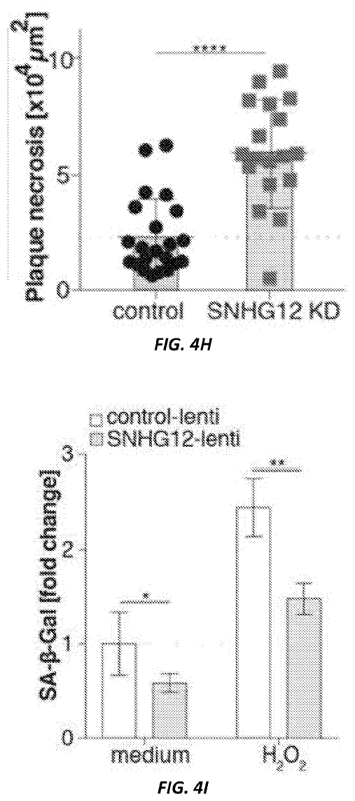

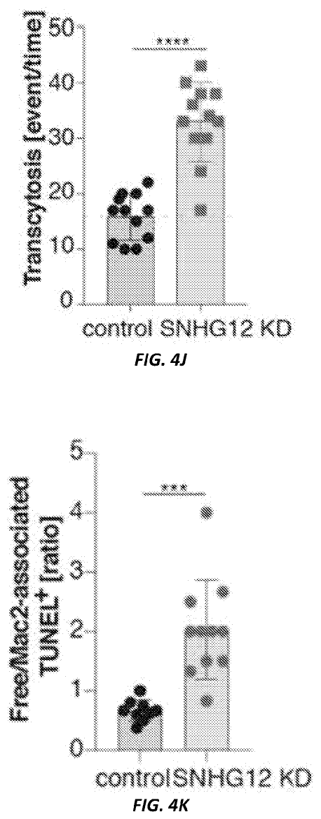

As a consequence of increased DNA damage, cells may exhibit impaired homeostatic control of functions important to lipoprotein entry or clearance of cellular debris or apoptotic cells (12). Consistent with this notion, knockdown of SNHG12 in ECs increased permeability via LDL transcytosis and impaired macrophage efferocytosis, or the ability to engulf apoptotic cells, a process implicated in the clearance of cells from the core of plaques (FIG. 4).

Oxidative stress induces genomic instability that can lead to DNA damage check point arrest, and if not appropriately repaired, to senescence and/or apoptosis (11). Most lesional DNA defects occur through the generation ROS via the NAD(P)H oxidase (44, 45). Herein, we demonstrate that NR, a clinical grade small molecule activator of NAD+ that serves as a precursor for NAPDH, which in turn activates a number of canonical pathways that reduce oxidative stress and hence DNA damage (46), completely rescued the effects on DNA damage, vascular senescence, and atherosclerosis. Because clinical trials are investigating the use of NR formulations for diverse chronic conditions including peripheral artery disease (ClinicalTrials.gov: NCT03743636), heart failure (ClinicalTrials.gov: NCT03423342), and cognitive function (ClinicalTrials.gov: NCT03562468), the findings in this study provide new mechanistic insights into the role of NR in vascular senescence applicable to these conditions. Furthermore, the findings that NR can rescue accelerated atherosclerosis, may inform new strategies to ameliorate a range of vascular occlusive disease states.

Our study builds upon the emerging roles of lncRNAs in the progression of atherosclerosis. For example, the lncRNAs LeXis and MeXis control lipid metabolism via LXR pathways (47, 48). Deficiency of lncRNA Malat1 accelerated atherosclerosis and triggered robust immune system dysregulation mediated by bone marrow-derived cells (49). Collectively, these studies highlight that lncRNAs exert profound regulation of key signaling pathways relevant to homeostasis in the vessel wall. In summary, we have identified lncRNA SNHG12 in atherosclerotic lesions as a homeostatic regulator of genomic stability by interaction with DNA-PK, a key mediator of the DDR. Knockdown of the lncRNA SNHG12 impairs DNA damage repair leading to lesional DNA damage, vascular senescence, and accelerated atherosclerosis independent of effects on lipid-lowering or lesional inflammation. Intravenous administration of the lncRNA SNHG12 reduced lesional DNA damage and plaque burden. Strategies aimed at restoring SNHG12 expression or facilitating SNHG12-DNA-PK interactions may provide a translational approach to limit DNA damage and vascular senescence applicable to a range of chronic disease states.

Diseases Associated with Malfunctions in DNA Damage Repair

The ability to repair DNA damage is an essential component of the genetic mechanisms conserving genomic fidelity. DNA damage may take several forms, including single- and double-strand breaks, inter- and intrastrand crosslinks and different kinds of base modifications. DNA damage may be the result of a variety of factors. Common exogenous sources of DNA damage include, chemical compounds and irradiation. Endogenous sources include spontaneous chemical conversion (e.g. deamination or depurination), the effect of oxygen and free radicals (causing base damage and DNA strand breaks), and malfunctions in DNA replication mechanisms (causing base mismatches and deletions). At the cellular level DNA damage may affect functions such as transcription, DNA replication, cell cycle, apoptosis and mutagenesis. At the phenotypic level this can lead to the development of diseases such as cancer and aging. Each cell has several complex methods in place to deal with both single base, and structural mismatches. Common repair pathways for double stranded breaks are homologous recombination based mechanisms. Another common mechanism for double-stranded DNA break repair is non-homologous end joining. The mechanisms of double-stranded break repair, and the diseases associated with them, have been reviewed by Khanna and Jackson “DNA double-strand breaks: signaling, repair and the cancer connection.” Nature Genetics March 2001; 27(3):247-254.

Non-homologous end joining (NHEJ) is a pathway that repairs double-strand breaks in DNA. NHJ is referred to as “non-homologous” because the break ends are directly ligated without the need for a homologous template. Ku is a dimeric protein complex that binds to DNA double-strand break ends and is required for the NHJ pathway of DNA repair. Ku is a heterodimer of two polypeptides, Ku70 (XRCC6) and Ku80 (XRCC5). The two Ku subunits form a basket-shaped structure that threads onto the DNA end. Once bound, Ku can slide down the DNA strand, allowing more Ku molecules to thread onto the end. In higher eukaryotes, Ku forms a complex with the DNA-dependent protein kinase catalytic subunit (DNA-PKcs) to form the full DNA-dependent protein kinase, DNA-PK. Ku is thought to function as a molecular scaffold to which other proteins involved in NHEJ can bind, orienting the double-strand break for ligation. A malfunction in DNA repair is a loss or reduction of function of any part of the repair pathway, and results in accumulation of mutations.

Malfunctions in DNA repair pathways have been implicated in a number of diseases, including cancer and aging. Exemplary diseases associated with DNA repair mechanisms include the following: atherosclerosis, heart failure, diabetes, hypertension, neurodegenerative disease, autoimmune disease, ataxia telangiectasia, aging, Bloom's Syndrome, immunodeficiency, Cockayne syndrome, Nijmegen breakage syndrome, Trichothiodystrophy, Fanconi Anaemia, Werner Syndrome, Li-Fraumeni syndrome, xeroderma pigmentosum, senescence, Hutchinson-Gilford progeria syndrome, and cancer, including breast cancer, lung cancer, and skin cancer, and toxicity from chemotherapeutic drugs. The present methods can be particularly useful in chemopreventative strategies that reduce the risk of developing cancer in subjects with a a genetic predisposition to develop cancer, e.g., subjects with BRCA1-related genetic predisposition for breast cancer; subjects who are carriers of rare familial adenomatous polyposis (FAP); or subjects with chronic inflammatory syndromes that are predisposed to cancer such as ulcerative colitis, Crohn's disease who are prone to colon cancer.

The present disclosure is based in part on the discovery that the SNHG12 lncRNA regulates DNA damage repair. Accordingly, in some embodiments, provided herein are methods of treating, or reducing risk of developing or progression of DNA damage or a disease associated with a malfunction in DNA repair. Generally, the methods include administering an amount of all or part of the SNHG12 lncRNA or a nucleic acid encoding SNHG12 lncRNA, as described herein, to a subject who is in need of, or who has been determined to be in need of, such treatment.

As used herein, “treating” a subject suffering from a disease associated with a malfunction in DNA repair response means administration to a patient by any suitable dosage regimen, procedure and/or administration route of a composition, device or structure with the object of achieving a desirable clinical/medical end-point, including but not limited to, stopping or slowing progression, reversing, or reducing the rate of DNA damage such that symptoms of a given disorder ameliorated.

As used in this context, to “treat” means to ameliorate at least one symptom or clinical parameter of disease associated with a malfunction in DNA repair. For instance, in the case of atherosclerosis, a treatment can result in stopping, slowing, reversing, or reducing of plaque formation. If the disease is cancer, symptoms (depending on the type) can include persistent cough or blood-tinged saliva, change in bowel habits, blood in the stool, anemia, breast lump or breast discharge, lumps in the testicles, a change in urination, swollen glands, changes in warts or moles, indigestion, weight gain or loss, night sweats, fever, sores, headaches, back pain, pelvic pain, bloating; a treatment can result in a reduction in any one or more of the symptoms, or in reduction in tumor size, number, growth rate, or metastatic potential.

Subjects

As used herein, the term “patient” or “subject” refers to members of the animal kingdom including but not limited to human beings and “mammal” refers to all mammals, including, but not limited to human beings and veterinary subjects such as cats, dogs, horses, pigs, cows, goats, and sheep.

In some embodiments, the present methods are used to treat subjects who have or are at risk (i.e., have a risk level above that of the general population or a relevant reference population) of developing a disease associated with malfunctions in DNA repair (e.g., as described above). Risk can be determined based on the presence or degree of known risk factors, including family history, history of smoking, history of exposure to environmental toxins known to cause DNA damage, and dietary habits (e.g., a history of consumption of high fat, high cholesterol foods). The methods can include identifying a subject for treatment using a method described herein, based on the presence (e.g., diagnosis) or risk of developing a disease associated with DNA damage. A subject at risk for or having such a disease can be readily recognized by one of ordinary skill in the art.

Atherosclerosis

Atherosclerosis has recently been shown to be associated with DNA damage, which affects both resident cells in the vessel wall and circulating cells that migrate into plaque (3-5). Therefore, in some embodiments, provided herein are methods of treating, or reducing risk of developing or progression of atherosclerosis. Generally, the methods include administering an amount of all or part of the SNHG12 lncRNA, as described herein, to a subject who is in need of, or who has been determined to be in need of, such treatment.

As used in this context, to “treat” means to ameliorate at least one symptom of atherosclerosis. Often, atherosclerosis results in formation and buildup of plaques on arterial walls, resulting eventually in narrowing of the blood vessel, with symptoms (e.g., angina pectoris) and acute coronary syndromes due to plaque breakup and arterial blockage; thus, a treatment can result in a reduction in formation and buildup of plaques on arterial walls and a reduction in level or risk of vessel narrowing and/or acute coronary syndromes (acute coronary thrombosis, myocardial infarction or sudden cardiac death).

Atherosclerotic Subjects

In some embodiments, the present methods are used to treat subjects who have or are at risk (i.e., have a risk level above that of the general population or a relevant reference population) of developing atherosclerosis. Risk can be determined based on the presence or degree of known risk factors, including family history, hypercholesterolaemia/hyperlipidemia (i.e., high levels as shown in Table A), diagnosis of diabetes mellitus, history of past or present cigarette smoking, hypertension (see Table B), dyslipoproteinaemia, and dietary deficiency of antioxidants (see, e.g., Burke et al., Circulation. 2002; 105:419-424).

TABLE 1
Cholesterol/LDL level categories

| Category | Total cholesterol | LDL cholesterol |
|---|---|---|
| desirable | Less than 200 | Less than 100; below 70 if coronary |
| | | artery disease is present |
| Borderline | 200-239 | 130-159 |
| High | 240 or higher | 160 or higher; 190 considered very high |

TABLE 2
Hypertension categories

| | systolic mm hg | | diastolic mm hg |
|---|---|---|---|
| Category | (upper number) | | (lower number) |
| Normotensive | less than 120 | and | less than 80 |
| Elevated | 120-129 | and | less than 80 |
| Hypertension stage 1 | 130-139 | or | 80-89 |
| Hypertension stage 2 | 140 or higher | or | 90 or higher |
| hypertensive crisis | higher than 180 | and/or | higher than 120 |

The American Heart Association (AHA) classification system categories include early stage lesions: initial type I, adaptive intimal thickening; and type II, fatty streak. Type III are transitional or intermediate lesions. Advanced plaques are categorized as type IV, atheromas; type V, fibroatheromas or atheromas with thick fibrous caps; and type VI, complicated plaques with surface defects, hematoma-hemorrhage, and/or thrombosis. In some embodiments, subjects treated using a method described herein have early stage atherosclerosis, e.g., type I or type II lesions, or have intermediate (type III) lesions. (Stary et al., Arterioscler Thromb. 1994; 14:840-856; Stary et al., Arterioscler Thromb Vasc Biol. 1995; 15:1512-1531). Alternatively, a modified classification system has been proposed, see Table C. (see Virmani et al., Arteriosclerosis, Thrombosis, and Vascular Biology. 2000; 20:1262-1275).

TABLE 3
Modified AHA Classification Based on Morphological Description*

| | | |
|---|---|---|
| Category | Description | Thrombosis |

Nonatherosclerotic intimal lesions

| | | |
|---|---|---|
| Intimal | The normal accumulation of smooth muscle | Absent |
| thickening | cells (SMCs) in the intima in the absence of | |
| | lipid or macrophage foam cells | |
| Intimal | Luminal accumulation of foam cells without a | Absent |
| xanthoma, or | necrotic core or fibrous cap. Based on animal | |
| “fatty streak” | and human data, such lesions usually regress. | |

Progressive atherosclerotic lesions

| | | |
|---|---|---|
| Pathological | SMCs in a proteoglycan-rich matrix with | Absent |
| intimal | areas of extracellular lipid accumulation | |
| thickening | without necrosis | |
| Erosion | Luminal thrombosis; plaque same as above | Thrombus mostly mural |
| | | and infrequently occlusive |
| Fibrous cap | Well-formed necrotic core with an overlying | Absent |
| atheroma | fibrous cap | |
| Erosion | Luminal thrombosis; plaque same as above; | Thrombus mostly mural |
| | no communication of thrombus with necrotic | and infrequently occlusive |
| | core | |
| Thin fibrous | A thin fibrous cap infiltrated by macrophages | Absent; may contain |
| cap atheroma | and lymphocytes with rare SMCs and an | intraplaque |
| | underlying necrotic core | hemorrhage/fibrin |
| Plaque rupture | Fibroatheroma with cap disruption; luminal | Thrombus usually |
| | thrombus communicates with the underlying | occlusive |
| | necrotic core | |
| Calcified | Eruptive nodular calcification with underlying | Thrombus usually |
| nodule | fibrocalcific plaque | nonocclusive |
| Fibrocalcific | Collagen-rich plaque with significant stenosis | Absent |
| plaque | usually contains large areas of calcification | |
| | with few inflammatory cells; a necrotic core | |
| | may be present. | |

*Adapted from Table 3 of Virmani et al., Arteriosclerosis, Thrombosis, and Vascular Biology. 2000; 20: 1262-1275.

In some embodiments, subjects treated using a method described herein have nonatherosclerotic intimal lesions, or have pathological intimal thickening with or without erosion. See, e.g. Ladich et al., Atherosclerosis Pathology, Updated: Sep. 12, 2016, available at reference.medscape.com/article/1612610-overview#showall; Virmani et al., Arteriosclerosis, Thrombosis, and Vascular Biology. 2000; 20:1262-1275; Bergheanu et al., Neth Heart J. 2017 April; 25(4): 231-242. In some embodiments, the subjects do not yet have plaque formation.

Additionally, in some embodiments, subjects include those without established atherosclerosis, e.g. at risk for atherosclerosis such as patients with diabetes who are at high risk for DNA damage in the vessel wall and atherosclerosis.

Small Nucleolar Host Gene-12 (SNHG12) Long Non-Coding RNA

As used herein, the term “nucleic acid” or “nucleic acid molecule” refers to any nucleic acid containing molecule, including but not limited to, DNA, RNA (e.g., long non coding RNA). The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethyl guanine, 2-methyladenine, 2-methylguanine, 3-methyl cytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

Lon non-coding RNAs (lncRNA) are functional RNA molecules that are not translated into a protein. Long ncRNAs are generally considered to be non-protein coding transcripts longer than about 200 nucleotides and have been shown to play roles in regulation of gene transcription, post-transcriptional regulation and epigenetic regulation (see, e.g., Guttman, M. et al., Nature., 2009, 458, 223-227).

Small Nucleolar Host Gene-12 (SNHG12), also known as LNC04080, is a lncRNA located at the p35.3 region on chromosome 1. It is 1,867 bases long and encodes four small nucleolar RNAs (SNORA66, SNORA61, SNORA16A, and SNORD99) from its spliced introns. Studies have implicated SNHG12 in a number of cancers, such as breast, gastric, osteosarcoma, and glioma and other cancer types. The altered expression of SNHG12 has been correlated with the viability, proliferation, metastasis, and invasion of tumor cells, impacting the prognosis and survival of cancer patients (Tamang, S et al., “SNHG12: An LncRNA as a Potential Therapeutic Target and Biomarker for Human Cancer”, Front Oncol. 2019; 9: 901). However, the previous investigations have shown nothing more than an association with cancer, lacking any demonstration of causality or evidence of a role in vivo.

The compositions useful in the present methods can include all or part of the SNHG12 lncRNA, or a nucleic acid sequence encoding the SNHG12 lncRNA.

In some embodiments, the sequence of SNHG12 lncRNA is or comprises: TCTAGAGCTAGCGAATTCCTTTCTCCCCGCCGCATTCCCGGTGTCGACTTACT AGCTGCAAGCCTCTGCCTGCCTTCCTGCGCGCCGTTCCCCGCTAGTCGCTGCT GCTGGCGCGCACTCGCCGGGTTTTTCCTCCCACGGCCTCGAGATGGTGGTGA ATGTGGCACGGAGGAGCCGGGCCTTCCAACCCGGTGGGCCCGAGCTCCGAAA GGCCCCCTCGGCAGTGAGAGGGGCGGGAGCCCGCGGGGGCCGCGCCCTTCTC TCGCTTCGGACTGCGCAACGCTGCGCTCTGGGCTGACAGGCGGATAAAACGG TCCCATCAAGACTGAGAAAAAGCACACCAGCTATTGGCACAGCGTGGGCAGT GGGGCCTACAGGATGACTGACTTAGTCTACAGAGATCCCGGCGTACTTAAGC AGATGAAGACTCTTAAGATGACAGAAGGTGATTTTTCTGGTGATCGAGGACT TCCGGGGTAATGACAGTGATGAAATGCAGGGGACCTGGTTGCCCCCAAGTTT CCTGGCAGTGTGTGATACTGAGGAGGTGAGCTTGTTTCTGGAGCTGTGCTTTA AGGTAAAGTTGATCAGCTTAATCCTCCTGATCCCTTTCCCATCGGATCTGAAC ACTGGTCTTGGTGGTCGTAAAAGGAGGAAAAGTAATAGTGAAGCTGGCCTAA ATGTTGTAATCTGGTATATGGCATGTGGGCTAGTTTCAGACAGGTTTCAGAGA TGGTTGGATCTCTGAAATTGTAAAATGAAGTATAATCTTAGGCTAAGGGAAG GATGCGTGTGAAGCTCTGGAGGTTGGTATAGTAATAGCTGACCTATTACTGC ATTTGGGAGGGATCTGTCATAGCTTCCTTGCCTCTTAATTAAGGGTGGTGTTT TTTTCTTTTAGATTCATGTTACATGTAAAGCTGTCCTCATTTGTGACTATGGAC CTATGGAGTTGGGACAATCTCTATGGGAAGCAGAAGGCAAGGACCCCGGTCA TTTTAGGTAGAAACAACAGCATGCTAATGCAAAAAATTATGCAGTGTGCTAC TGAACTTCAGAGGTGATCAATAAAAGAAGAATAAAAAGACTAATAAAAGTA GAATTCTGATCAGGATCC (SEQ ID NO: 1); i.e. full length SNHG12 lncRNA.

In some embodiments, the sequence of SNHG12 lncRNA is or comprises:

(SEQ ID NO: 2)
TCGGATCTGAACACTGGTCTTGGTGGTCGTAAAAGGAGGAAAAGTAATAG
TGAAGCTGGCCTAAATGTTGTAATCTGGTATATGGCATGTGGGCTAGTTT
CAGACAGGTTTCAGAGATGGTTGGATCTCTGAAATTGTAAAATGAAGTAT
AATCTTAGGCTAAGGGAAGGATGCGTGTGAAGCTCTGGAGGTTGGTATAG
TAATAGCTGACCTATTACTGCATTTGGGAGGGATCTGTCATAGCTTCCTT
GCCTCTTAATTAAGGGTGGTGTTTTTTTCTTTTAGATTCATGTTACATGT
AAAGCTGTCCTCATTTGTGACTATGGACCTATGGAGTTGGGACAATCTCT
ATGGGAAGCAGAAGGCAAGGACCCCGGTCATTTTAGGTAGAAACAACAGC
ATGCTAATGCAAAAAATTATGCAGTGTGCTACTGAACTTCAGAGGTGATC
AATAAAAGAAGAATAAAAAGACTAATAAAAGTAGAATTCTGATCAGGATC
CTCTAGAGCTAGCGAATTCCTTTCTCCCCGCCGCATTCCCGGTGTCGACT
TACTAGC.

In some embodiments, the sequence of SNHG12 lncRNA is or comprises: nucleotide residues 598 to 1117 position of SEQ ID NO: 1, and optionally an additional 38 nucleotides from exon 1, e.g., SEQ ID NO: 2.

In some embodiments, provided herein are synthetic SNHG12 lncRNA (e.g., SEQ ID NO: 1 or 2 or sequences with at least 40, 45, 50, 55, 65, 70, 75, 80, 85, 90, 95, or 99% sequence identity with SEQ ID NO: 1 or 2)). The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at gcg. com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

Viral Vectors

Viral vectors for use in the present methods and compositions include recombinant retroviruses, adenovirus, adeno-associated virus, alphavirus, and lentivirus.

A preferred viral vector system useful for delivery of nucleic acids in the present methods is the adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. Moreover, the AAV capsid can be genetically engineered to increase transduction efficient and selectivity, e.g., biotinylated AAV vectors, directed molecular evolution, self-complementary AAV genomes and so on. In some embodiments, AAV1 is used. In some embodiments, AAV 8 is used. In some embodiments, AAV 9 is used.

Alternatively, retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Alphaviruses can also be used. Alphaviruses are enveloped single stranded RNA viruses that have a broad host range, and when used in gene therapy protocols alphaviruses can provide high-level transient gene expression. Exemplary alphaviruses include the Semliki Forest virus (SFV), Sindbis virus (SIN) and Venezuelan Equine Encephalitis (VEE) virus, all of which have been genetically engineered to provide efficient replication-deficient and -competent expression vectors. Alphaviruses exhibit significant neurotropism, and so are useful for CNS-related diseases. See, e.g., Lundstrom, Viruses. 2009 June; 1(1): 13-25; Lundstrom, Viruses. 2014 June; 6(6): 2392-2415; Lundstrom, Curr Gene Ther. 2001 May; 1(1):19-29; Rayner et al., Rev Med Virol. 2002 September-October; 12(5):279-96.

Where a vector, virus, or naked DNA is delivered, regulatory sequences controlling expression of the lncRNA should also be included, e.g., a promoter; an enhancer sequence, e.g., 5′ untranslated region (UTR) and/or a 3′ UTR; a polyadenylation site; an insulator sequence; or another sequence that increases the expression of the lncRNA.

### Making and Using the Nucleic Acids

The present methods can include delivery of RNA, e.g., naked RNA, or delivery of a nucleic acid encoding the lncRNA, e.g., a cDNA, vector, or virus encoding the lncRNA. The nucleic acids used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including, for e.g., in vitro bacterial, fungal, mammalian, yeast, insect, or plant cell expression systems. Nucleic acid sequences that can be used in any of the methods described herein can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example, as described in Sambrook et al., Molecular Cloning: A Laboratory Manual., 1989; Coffin et al., Retroviruses, 1997; and “RNA Viruses: A Practical Approach” (Alan J. Cann, Ed., Oxford University Press, 2000). As will be apparent to one of ordinary skill in the art, a variety of suitable vectors are available for transferring nucleic acids (e.g., a nucleic acid comprising all or a part of SEQ ID NO: 1 or 2) into cells.

The selection of an appropriate vector to deliver nucleic acids and optimization of the conditions for insertion of the selected expression vector into the cell, are within the scope of one of ordinary skill in the art without the need for undue experimentation. Viral vectors include a nucleotide sequence having sequences for the production of recombinant virus in a packaging cell. Viral vectors expressing nucleic acids (e.g., a sequence comprising all or a part of SEQ ID NO: 1 or 2) can be constructed based on viral backbones including, but not limited to, a retrovirus, lentivirus, adenovirus, adeno-associated virus, herpes simplex virus, pox virus, or alphavirus. The recombinant vectors capable of expressing a nucleic acid (e.g., a nucleic acid comprising all or part of SEQ ID NO: 1 or 2) can be delivered as described herein, and persist in target cells (e.g., stable transformants).

In addition to viral transfer methods, such as those illustrated above, non-viral methods can also be employed to cause expression of a nucleic acid compound described herein. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther. 7(22):1896-905 (2000); or Tam et al., Gene Ther. 7(21):1867-74 (2000). Other embodiments include Crispr-Cas9/Cas13 based systems to modify lncRNA expression (see, e.g., ncbi.nlm.nih.gov/pubmed/30045635).

Nucleic acid sequences comprising all or a part of SEQ ID NO: 1 or 2 can be synthesized in vitro by well-known chemical synthesis techniques, as described in, e.g., Adams, J. Am. Chem. Soc. 105:661, 1983; Belousov, Nucleic Acids Res. 25:3440-3444, 1997; Frenkel, Free Radic. Biol. Med. 19:373-380, 1995; Blommers, Biochemistry 33:7886-7896, 1994; Narang, Meth. Enzymol. 68:90, 1979; Brown, Meth. Enzymol. 68:109, 1979; Beaucage, Tetra. Lett. 22:1859, 1981; and U.S. Pat. No. 4,458,066.

Nucleic acid sequences comprising all or a part of SEQ ID NO: 1 or 2 can be stabilized against nucleolytic degradation, nuclease stability, decrease the likelihood of triggering an innate immune response, lower the incidence of off-target effects, and/or improve pharmacodynamics relative to non-modified molecules so as to increase potency and specificity, such as by the incorporation of a modification, e.g., a nucleotide modification. For example, nucleic acid sequences (e.g., nucleic acids comprising all or a part of SEQ ID NO: 1 or 2) can include a phosphorothioate, boranophosphate, or 4′-thio-ribose.

As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (T-O-DMAEOE), (2′-O—N-methylacetamido (2′-O-NMA), 2′-O-(2′-methoxyethyl), 2′-O-alkyl, 2′-O-alkyl-O-alkyl, 2′-amino, T-deoxy-T-fluoro-b-D-arabinonucleic acid. As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., nucleic acid analogues in which the ribose ring is “locked” by a methylene bridge connecting the 2′-o atom and the 4′-C atom (see, e.g., Kaupinnen et al., Drug Disc. Today 2(3):287-290, 2005; Koshkin et al., J. Am. Chem. Soc. 120(50):13252-13253, 1998). In some embodiments, the RNA is modified by pseudouridine and/or 5-methylcytidine substitution. For additional modifications see Kaczmarek et al., Genome Med. 2017; 9: 60, US 2010/0004320, US 2009/0298916, and US 2009/0143326.

The nucleic acid can be further modified at the 5′ end by capping the end, which is known in the art. At the end of transcription, the 5′ end of the RNA transcript contains a free triphosphate group since it was the first incorporated nucleotide in the chain. Capping replaces the triphosphate group with another structure called the “5′ cap”. Suitable 5′ caps include methylated guanosine. In some embodiments, a N7-methyl guanosine is connected to the 5′ nucleotide through a 5′ to 5′ triphosphate linkage, typically referred to as m7G cap, m7 Gppp, or cap 0 in the literature. An additional methylation on the 2′O position of the initiating nucleotide generates Cap 1, or referred to as m7GpppNm-, where Nm denotes any nucleotide with a 2′O methylation. See, e.g., Kaczmarek et al., Genome Med. 2017; 9: 60.

In some embodiments, the nucleic acid is delivered using a liposome or nanoparticle, e.g., by attachment to or encapsulation within a biocompatible nanoparticle. The nanoparticles can be tagged with antibodies against cell surface antigens of the target tissue. In some embodiments, the nucleic acid is modified to with a N-acetylgalactosamine GalNAc-conjugate approach (include wherever lipid nanoparticle is mentioned). In some embodiments, the nucleic acid is conjugated with PEI or an antibody targeted to the tunica intima or other relevant cell types.

Examples of biocompatible nanoparticles include liposomes and polymeric nanoparticles. As used herein, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged nucleic acid molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

Polymeric nanoparticles for use in the present methods and compositions can comprise cationic polymers, such as amine-containing polymers, poly-L-lysine, polyamidoamine, and polyethyleneimine, chitosan, poly(β-amino esters). In some embodiments the cationic polymers electrostatically condense the negatively charged RNA into nanoparticles. See, e.g., Pack et al., Nat Rev Drug Discov. 2005 July; 4(7):581-93; Kaczmarek et al., Genome Med. 2017; 9: 60.

Techniques for the manipulation of nucleic acids used to practice this invention, such as, e.g., subcloning, labeling probes (e.g., random-primer labeling using Klenow polymerase, nick translation, and amplification), sequencing, hybridization, and the like are well described in the scientific and patent literature, see, e.g., Sambrook et al., Molecular Cloning; A Laboratory Manual 3d ed., 2001; Current Protocols in Molecular Biology, Ausubel et al., Eds. (John Wiley & Sons, Inc., New York, 2010); Kriegler, Gene Transfer and Expression: A Laboratory Manual, 1990; Laboratory Techniques in Biochemistry and Molecular Biology: Hybridization with Nucleic Acid Probes, Part I. Theory and Nucleic Acid Preparation, Tijssen, Ed., Elsevier, N.Y., 1993.

In certain embodiments, gene therapy, utilizing zinc finger recombinase fusion proteins, is used to site-specifically exchange the promoter of SNHG12 with a promoter that has constitutive or higher level expression. In some embodiments, the Tet promoter, for example, is used, and after recombining in the targeted cells, the gene is turned on by administering tetracycline to the subject. One exemplary alternative of this approach is to introduce the SNHG12 gene behind the native promoter with the desired level of expression. Another exemplary alternative is using CrispR to activate upstream sequences to SNHG12; or by using small molecule activators.

In some embodiments, the expression of negative regulators of SNHG12 are reduced and/or inhibited, thereby increasing the expression of SNHG12. Similarly, in certain embodiments, degradation of SNHG12 can be reduced by administering agents that inhibit SNHG12 degradation pathways.

### Pharmaceutical Compositions, Dosing, Administration

The methods described herein can include the administration of pharmaceutical compositions and formulations that include the nucleic acid sequences described herein (e.g., nucleic acids comprising all or a part of, or encoding all or part of, the sequence of SEQ ID NO: 1 or 2).

As used herein, the term “effective amount” refers to the amount of a composition sufficient to effect beneficial or desired results. An effective amount can be administered in one or more administrations, applications or dosages and is not intended to be limited to a particular formulation or administration route.

The term “compound” refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using screening methods. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of disease (e.g., atherosclerosis).

In some embodiments, the compositions are formulated with a pharmaceutically acceptable carrier. The pharmaceutical compositions and formulations can be administered parenterally, intramuscularly, intravitreally, subcutaneously, arterially, intravenously (IV, i.v.), topically, orally, or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005. Those of skill in the art understand that the formulations and/or routes of administration of the various agents or therapies used may vary.

The nucleic acids can be administered alone or as a component of a pharmaceutical formulation (composition). The compounds may be formulated for administration, in any convenient way for use in human or veterinary medicine. Wetting agents, emulsifiers, and lubricants, such as sodium lauryl sulfate and magnesium stearate, as well as coloring agents, release agents, coating agents, sweetening, flavoring, perfuming agents, preservatives, and antioxidants can also be present in the compositions. Formulations of the compositions that may be used in the methods described herein include those suitable for intradermal, inhalation, intramuscular, subcutaneous, arterial, intravenous, oral/nasal, topical, parenteral, rectal, and/or intravaginal administration. The formulations may conveniently be presented in unit dosage form and may be prepared by any methods well known in the art of pharmacy. The amount of active ingredient (e.g., a nucleic acid sequence described herein) which can be combined with a carrier material to produce a single dosage form will vary depending upon the host being treated, the particular mode of administration, e.g., intravenous or inhalation. The amount of active ingredient which can be combined with a carrier material to produce a single dosage form will generally be that amount of the compound which produces a therapeutic effect Pharmaceutical formulations of this invention can be prepared according to any method known to the art for the manufacture of pharmaceuticals. Such drugs can contain sweetening agents, flavoring agents, coloring agents, and preserving agents. A formulation can be admixed with nontoxic pharmaceutically acceptable excipients which are suitable for manufacture. Formulations may include one or more diluents, emulsifiers, preservatives, buffers, excipients, etc., and may be provided in such forms as liquids, powders, emulsions, lyophilized powders, sprays, creams, lotions, controlled release formulations, tablets, pills, gels, on patches, in implants, etc.

In some embodiments, the pharmaceutical composition comprising all or a part of the sequence of SEQ ID NO: 1 or 2 also includes a gene enhancer that increases the expression of SNHG12. In some embodiments the all or a part of the sequence of SEQ ID NO: 1 or 2 may be co-administered with a gene enhancer.

In some embodiments, provided herein is a pharmaceutical composition capable of increasing SNHG12 expression. In some embodiments the all or a part of the sequence of SEQ ID NO: 1 or 2 may be co-administered with a compound capable of increasing SNHG12 expression.

Dosage regimens may be adjusted to provide the optimum desired response (e.g., a therapeutic or prophylactic response). For example, a single bolus may be administered, several divided doses may be administered over time, or the composition may be administered continuously or in a pulsed fashion with doses or partial doses being administered at regular intervals, for example, ever 10, 15, 20, 30, 45, 60, 90, or 120 minutes, every 2 through 12 hours daily, or every other day, etc. be proportionally reduced or increased as indicated by the exigencies of the therapeutic situation. In some instances, it may be especially advantageous to formulate parenteral compositions in dosage unit form for ease of administration and uniformity of dosage. The specification for the dosage unit forms of the invention are dictated by and directly dependent on (a) the unique characteristics of the active compound and the particular therapeutic or prophylactic effect to be achieved, and (b) the limitations inherent in the art of 20 compounding such an active compound for the treatment of sensitivity in individuals.

### EXAMPLES

The invention is further described in the following examples, which do not limit the scope of the invention described in the claims.

### Materials and Methods

The following materials and methods were used in the Examples below.

### Study Design

The main goal of this study was to identify dynamically regulated lncRNAs with progression of atherosclerosis. Among them, we identified the lncRNA SNHG12 as a highly abundant and evolutionary conserved lncRNA from mouse to pig to human. Loss-of-function studies were performed with modified ASOs (gapmeRs) to assess the role of SNHG12 in the progression of atherosclerosis. In vivo experiments for loss-of-function studies were performed in atherosclerotic prone LDLR^{-/-} mice in conjunction with HCD by tail vein administration of gapmeRs twice per week over the course of 12 weeks in the presence or absence of NR. For gain-of-function experiments SNGH12 RNA was delivered via the same route in ApoE−/− mice that were 12 weeks of age at the start of the study. The study was performed over the course of 6 weeks on HCD. Experimental groups included at least 10 mice per group to robustly identify alterations in disease progression, and mice were randomly assigned to each group. All measurements were blinded. GapmeR-mediated knockdown and lentiviral overexpression in human endothelial cells and human primary macrophages was performed to verify the evolutionary conserved function of SNHG12. For in vitro studies, experiments included a minimum of n=3 independently performed replicates. Expression data from human and pig carotid cross sections were used to assess the atherosclerosis-specific expression profile of SNHG12. No data points were excluded as outliers.

### Animal Studies

All protocols concerning animal use were approved by the Institutional Animal Care and Use Committee at Brigham and Women's Hospital and Harvard Medical School, Boston, Mass. and conducted in accordance with the National Institutes of Health Guide for the Care and Use of Laboratory Animals. Studies were performed in LDLR^{-/-} mice (Jackson Laboratory, Stock#: 002207), ApoE^{-/-} mice 12 weeks old (Jackson Laboratory, Stock#:002052), or in C57B1/6 mice (Charles River, Strain code#027).

### Efferocytosis

Efferocytosis assay was performed as described in (50). Briefly, 5×10⁶ cells/mL Jurkat cells were labeled with 5 μM AM Calcein (Invitrogen, LS-H2452-50). After 2 hrs incubation, cells were washed and irradiated with UV (150 mJ/cm²) with an open lid, followed by another 2 hrs incubation before apoptotic cells were added in a 1:1 ratio to gapmeR-transfected primary macrophages. After several rounds of gentle washing, macrophages were counted positive for internalized green apoptotic bodies if they contained >3 μm clusters of green dots. Quantification was performed from four images with a total of 400 macrophages. For quantification of in vivo efferocytosis, macrophages were stained using rat anti-Mac2 (Cadarlane, CL8942AP, 1:100), as described below for immunofluorescence. TUNEL protocol was performed based on the manufacturer's protocol (Roche, In situ cell death detection kit, TMR red). The ratio of macrophage-free TUNEL over macrophage-associated TUNEL signaling was calculated as described (29).

### En Face RNA Isolation

RNA from the lesser and greater curvature (LC, GC) was isolated from C57B1/6 mice using nitrocellulose slides (ONCYTE NOVA). To this end, aortas were isolated and cut for areas of LC or GC and placed with the tunica intima side towards the nitrocellulose side for 15 min at RT (n=3 mice were pooled to represent n=1) (51, 52). After removing the aorta from the slide, RNA lysis buffer was directly added to the slide, followed by RNA isolation (QIAGEN, RNeasy Plus Micro Kit, #74034).

### Evans Blue Extravasation

In vivo permeability in arteries with Evans Blue extravasation was performed as described (27). Briefly, a cone-shape polyethylene constrictive cuff (CC) was placed in the left common carotid artery (LCCA) and secured by a circumferential suture. Evans Blue extravasation was determined following 24 hrs post-surgery. C57B1/6 mice were injected (i.v.) with either SNHG12-gapmeRs or control-gapmeRs (7.5 mg/kg) or with in vitro transcribed SNHG12 RNA (15 μg/injection) (as described below in MST assay) on two constitutive days followed by CC surgery on day 3. Downstream Evans Blue area of the CC elongation in longitudinal cross sections of the LCCA was compared to corresponding controls.

### Human Atherosclerotic Specimens

Frozen sections were prepared from human normal carotid arteries and carotid atherosclerotic lesions that were obtained from the Division of Cardiovascular Medicine, Brigham and Women's Hospital in accordance with the Institutional Review Board-approved protocol for use of discarded human tissues (protocol #2010-P-001930/2).

### Immunohistology and Characterization of Atherosclerotic Lesions

To quantify atherosclerosis in LDLR^{-/-} mice that were placed on HCD (Research Diets Inc., D12108C), aortic roots and aortic arch were embedded in OCT and frozen at −80° C. Serial cryostat sections (6 μm) were prepared using tissue processor Leica CM3050. Lesion characterizations, including Oil Red 0 (ORO) staining of the thoracic-abdominal aorta and aortic root and staining for macrophages (anti-Mac3, BD Pharmingen, 553322, 1:900) T cells (anti-CD4, BD Pharmingen, 553043, 1:90; anti-CD8, Chemicon, CBL1318, 1:100), and vascular smooth muscle cells (SM-α-actin, Sigma, F-3777, 1:500), were performed as previously described (53, 54). The staining area was measured using Image-Pro Plus software, Media Cybernetics, and CD4⁺ and CD8⁺ cells were counted manually.

Intimal RNA isolation from Aorta Tissue

Isolation of intimal RNA from aorta was performed as previously described in (54, 55). Briefly, aortas were carefully flushed with PBS, followed by intima peeling using TRIzol reagent (Invitrogen, 15596018). TRIzol was flushed for 10 sec-10 sec pause—another 10 sec flushed and collected in an Eppendorf tube (˜300-400 μL total) and snap frozen in liquid nitrogen.

### LDL Transcytosis Assay

LDL transcytosis assay was performed as previously described (56). Briefly, total internal reflection fluorescence (TIRF) microscopy uses an evanescent wave to illuminate just the proximal ˜100 nm of the cell, thereby facilitating selective imaging of the basal membrane of a live EC with minimal confounding from the overlying cytoplasm and apical surface. Confluent human coronary artery HCAEC monolayers were exposed to a fluorophore-tagged ligand added to the apical cell surface while the basal membrane of the cell was imaged by TIRF. Cytoplasmic vesicles undergoing exocytosis with the basal membrane were directly visualized and quantified. TIRF microscopy was performed on a Leica DMi8 microscope with 63×/1.47 (O) objectives, 405 nm, 488 nm, 561 nm and 637 nm laser lines, 450/50, 525/50, 600/50, 610/75 and 700/75 emission filters and run with Quorum acquisition software (Quorum). Microscope settings were kept constant between conditions. Briefly, cells at 100% confluency were placed in a live cell imaging chamber and treated with 20 μg/mL DiI-LDL in cold HPMI media for 10 min at 4° C. to allow apical membrane-binding. Following membrane binding, cells were washed twice with cold PBS+ to remove unbound ligand and room temperature HPMI was added. Cells were incubated on the live cell imaging stage at 37° C. for two minutes before initial image acquisition. Confluent regions of the monolayer were selected by viewing the number of nuclei in the DAPI field of view after staining with NucBlue Live ReadyProbes Reagent (Thermo Fisher) and TIRF microscopy of the basal membrane was performed to visualize exocytosis. For each coverslip, 10-15 videos of 150 frames (100 ms exposure) were captured. Image analysis was performed using a custom MATLAB single particle-tracking algorithm (56).

### Liquid Chromatography-Mass Spectrometry (LC-MS/MS)

LC-MS/MS was performed as previously described (57). Briefly, lncRNA pulldown of SNHG12 or LacZ purified samples were reduced with 10 mM DTT for 30 min at 56° C. in the presence of 0.1% RapiGest SF (Waters). Cysteines were alkylated with 22.5 mM iodoacetamide for 20 min at room temperature in the dark. Samples were digested overnight at 37° C. with trypsin. Rapigest was then cleaved according to manufacturer's instructions and peptides purified by reversed phase and strong cation exchange chromatography. Peptides were loaded onto a precolumn (4 cm POROS 10R2, Applied Biosystems), resolved on a self-packed analytical column (12 cm Monitor C18, Column Engineering) after gradient elution (NanoAcquity UPLC system, Waters; 5%-35% B in 90 min; A=0.2M acetic acid in water, B=0.2M acetic acid in acetonitrile), and introduced to the MS (TripleTOF 5600, ABSciex, Framingham, Mass.) by ESI (spray voltage=2.2 kV). The mass spectrometer was programmed to perform data-dependent MS/MS (unit resolution, m/z 100-2000) on the 20 most abundant precursors in each MS1 scan (m/z 300-2000; accumulation time=0.5 seconds; threshold=70 counts; charge state=2+ to 5+) using a rolling collision energy. After MS/MS, each precursor was excluded for 25 seconds. Raw data were converted to .mgf using ABSciex MSDataConverter; precursor and product ions were recalibrated using a linear equation derived from fitting experimentally observed masses obtained in an initial low mass tolerance database search. Recalibrated data were matched to peptide sequences in a forward/reversed human NCBI refseq database using Mascot version 2.4.1. Search parameters included trypsin specificity with up to two missed cleavages, fixed carbamidomethylation (C, +57 Da) and variable oxidation (M, +16 Da). Precursor and product mass tolerances were 12 ppm and 25 mmu. Protein hits with FDR<0.1 from SNHG12-specific pulldown were compared to negative control (LacZ) (n=2, with two technical replicates).

### Non-Homologous End Joining (NHEJ) Repair by FACS

NHJ ability was assessed as previously described (22). Lentivirus was produced in 293T cells as described above under “Lentivirus production and transduction” for pDRR (double strand break repair reporter (pLCN DSB Repair Reporter, Addgene, #98895) and pCBASceI (Addgene, #26477). HUVECs were transduced in 10 cm dish with pDRR without Geneticin selection, because >85% were GFP positive cells after 4-5 days. Cells were transfected with GapmeRs, siRNA or transduced with lentivirus for SNHG12 and 24-48 hrs after, cells were transduced with lentivirus for pCBASceI. FACS analysis for GFP positive cells was performed 48-72 hrs post pCBASceI transduction.

### Pig Atherosclerotic Samples

The study protocol included 15 male hypercholesterolemic Yorkshire swine that were placed on an HCD for up to 60 weeks. Detailed sectioning of 3-mm coronary artery segments was performed so that the gene sequencing samples were derived from the exact same portions of the coronary artery plaques used for the histology and immunohistochemistry analyses. Histology and IHC analyses included H & E, van Gieson elastin staining, smooth muscle cell α-actin, oil red-O staining (ORO), picrosirius red staining, CD31 and CD45 cells as described in (58)

### Protein Coding Potential

In silico coding potential assessment tool (CPAT) online algorithm was used for prediction of coding potential (59). For in vitro validation of peptide coding potential, SNHG12 mouse or human transcripts were cloned upstream of p3×FLAG-CMV-14 expression vector (Sigma, E7908) using EcoRI restriction site. 293T cells were transfected with 500 ng plasmid using Lipofectamine 2000 (Invitrogen) and protein lysate was isolated 72 hrs post-transfection, followed by immunoblotting for FLAG Tag (Cell Signaling, 8146).

### RNA Synthesis, Modification and Injection

10 μg of linearized and purified T7 vector with the cassette for LacZ or SNHG12 was used for 1× T7 RNA polymerase transcription reaction (Promega, RiboMax™ Large Scale RNA, P1300) based on the manufacturer's protocol. Following 4 hrs incubation at 37° C., RNA was purified by standard phenol:chloroform isolation method and resuspended in 140 μL RNAase-free water. After 5 min at 65° C., RNA was capped and 2′-O-Methylated (NEB, #M0366) based on the manufacturer's protocol before purification using a column-based approach (QIAGEN, RNeasy Plus Universal Midi Kit, #73442) and stored in −80° C. RNA with concentration of 2-3 μg/μL was injected i.v. using (Polyplus, in vivo jetPEI, #201-50G). Briefly, 15 μg RNA was diluted in water and glucose (final conc.5%) in a volume of 100 μL. This mixture was combined with a premixed cocktail of 6.4 μL jetPEI, 43.6 μL, RNAase-free water, and 50 μL 10% glucose (final conc. 5%) and incubated for 15 min at RT before administrated (200 μL, volume total).

### RNA-Seq Analysis

RNA-Seq analysis was performed after ribodepletion and standard library construction using Illumina HiSeq2500 V4 2×100 PE (Genewiz, South Plainfield, N.J.). All samples were processed using an RNA-seq pipeline implemented in the bcbio-nextgen project (https://bcbio-nextgen.readthedocs.org/en/latest/). Raw reads were examined for quality issues using FastQC (http://www.bioinformatics.babraham.ac.uk/projects/fastqc/) to ensure library generation and sequencing were suitable for further analysis. Trimmed reads were aligned to UCSC build mm10 of the Mouse genome, augmented with transcript information from Ensembl release 79 using STAR (60). Alignments were checked for evenness of coverage, rRNA content, genomic context of alignments (for example, alignments in known transcripts and introns), complexity and other quality checks using a combination of FastQC, Qualimap (61). Counts of reads aligning to known genes were generated by featureCounts (62). Differential expression at the gene level were called with DESeq2 (63). The total gene hit counts and CPM values were calculated for each gene and for downstream differential expression analysis between specified groups was performed using DESeq2 and an adapted DESeq2 algorithm, which excludes overlapping reads, called no-overlapping reads (NOR). Genes with adjusted FDR<0.05 and log 2fold-change (1.5) were called as differentially expressed genes for each comparison. Mean quality score of all samples was 35.67 within a range of 40,000,000-50,000,000 reads per sample. All samples had at least >70% of mapped fragments over total.

### Sequence Data1. Sequences Used for Cloning.

human SNHG12-205 (ENST00000470977.6)
(SEQ ID NO: 1)
TCTAGAGCTAGCGAATTCCTTTCTCCCCGCCGCATTCCCGGTGTCGACTT
ACTAGCTGCAAGCCTCTGCCTGCCTTCCTGCGCGCCGTTCCCCGCTAGTC
GCTGCTGCTGGCGCGCACTCGCCGGGTTTTTCCTCCCACGGCCTCGAGAT
GGTGGTGAATGTGGCACGGAGGAGCCGGGCCTTCCAACCCGGTGGGCCCG
AGCTCCGAAAGGCCCCCTCGGCAGTGAGAGGGGCGGGAGCCCGCGGGGGC
CGCGCCCTTCTCTCGCTTCGGACTGCGCAACGCTGCGCTCTGGGCTGACA
GGCGGATAAAACGGTCCCATCAAGACTGAGAAAAAGCACACCAGCTATTG
GCACAGCGTGGGCAGTGGGGCCTACAGGATGACTGACTTAGTCTACAGAG
ATCCCGGCGTACTTAAGCAGATGAAGACTCTTAAGATGACAGAAGGTGAT
TTTTCTGGTGATCGAGGACTTCCGGGGTAATGACAGTGATGAAATGCAGG
GGACCTGGTTGCCCCCAAGTTTCCTGGCAGTGTGTGATACTGAGGAGGTG
AGCTTGTTTCTGGAGCTGTGCTTTAAGGTAAAGTTGATCAGCTTAATCCT
CCTGATCCCTTTCCCATCGGATCTGAACACTGGTCTTGGTGGTCGTAAAA
GGAGGAAAAGTAATAGTGAAGCTGGCCTAAATGTTGTAATCTGGTATATG
GCATGTGGGCTAGTTTCAGACAGGTTTCAGAGATGGTTGGATCTCTGAAA
TTGTAAAATGAAGTATAATCTTAGGCTAAGGGAAGGATGCGTGTGAAGCT
CTGGAGGTTGGTATAGTAATAGCTGACCTATTACTGCATTTGGGAGGGAT
CTGTCATAGCTTCCTTGCCTCTTAATTAAGGGTGGTGTTTTTTTCTTTTA
GATTCATGTTACATGTAAAGCTGTCCTCATTTGTGACTATGGACCTATGG
AGTTGGGACAATCTCTATGGGAAGCAGAAGGCAAGGACCCCGGTCATTTT
AGGTAGAAACAACAGCATGCTAATGCAAAAAATTATGCAGTGTGCTACTG
AACTTCAGAGGTGATCAATAAAAGAAGAATAAAAAGACTAATAAAAGTAG
AATTCTGATCAGGATCC
mouse SNHG12-206 (ENSMUST00000153474.8)
(SEQ ID NO: 3)
TCTAGAGCTAGCGAATTCTTTCTCGCTTCATCCGCGGTCCCTGTCTGTTT
TCGTTATGGCGCCTTGTACTTCTACCCAACGTTGCCCGCCCCCCATCTTC
CCAGCCCACCAGCTCCGTCCGCCTCTCCGGATGATTCGTGAGATACCGAG
CCTGCCGGGAAGGGACCGGATTTTTCCGTCTGGTCCAAGGAAGCACGGGT
TATGGCCACATGCGCAGTGATAACTCAGGGCCCCACGCCTTTTGACCCCT
GTTGATGAAGATTGTGAGGAGGGAGACCAGAAGATAATGGACAACGATAC
AGCAGAAGGGTCTGGTTACACTTGAGCTTATTTCTTGGAAGAGTCTGAGA
AGACGGCCTGTCAGGCTCCTAACACTAAGGGCCATGTAACCAGTGAAGCA
GCCATTATATATGATTTGGTCTACACTTGTTGGAAAAATTCCACAAGAGG
ATCTCCTGGAAGCTAGCAGAAGTTTTTGGCTTGTACATTGCCAGGCAGCC
ATTGGAATCGGAGCTGCAGTCAGATTAAGGCCAGCCTGGCCTACATTGAG
AAACCTCATTTTGGGAAAGTGAAATACTTTGTCAATTAACATGCAGTTGG
TTCAATAAAGACTTTTGGAAAGTGGAATTCTGATCAGGATCC
human SNHG12 deletion domain 1
(SEQ ID NO: 4)
CTTTCTCCCCGCCGCATTCCCGGTGTCGACTTACTAGCTGCAAGCCTCTG
CCTGCCTTCCTGCGCGCCGTTCCCCGCTAGTCGCTGCTGCTGGCGCGCAC
TCGCCGGGTTTTTCCTCCCACGGCCTCGAGATGGTGGTGAATGTGGCACG
GAGGAGCCGGGCCTTCCAACCCGGTGGGCCCGAGCTCCGAAAGGCCCCCT
CGGCAGTGAGAGGGGCGGGAGCCCGCGGGGGCCGCGCCCTTCTCTCGCTT
CGGACTGCGCAACGCTGCGCTCTGGGCTGACAGGCGGATAAAACGGTCCC
ATCAAGACTGAGAAAAAGCACACCAGCTATTGGCACAGCGTGGGCAGTGG
GGCCTACAGGATGACTGACTTAGTCTACAGAGATCCCGGCGTACTTAAGC
AGATGAAGACTCTTAAGATGACAGAAGGTGATTTTTCTGGTGATCGAGGA
CTTCCGGGGTAATGACAGTGATGAAATGCAGGGGACCTGGTTGCCCCCAA
GTTTCCTGGCAGTGTGTGATACTGAGGAGGTGAGCTTGTTTCTGGAGCTG
TGCTTTAAGGTAAAGTTGATCAGCTTAATCCTCCTGATCCCTTTCCCATC
GGATCTGAACACTGGTCTTGGTGGTCGTAAAAGGAGGAAAAGTAATAGTG
AAGCTGGCCTAAATGTTGTAATCTGGTATATGGCATGTGGGCTAGTTTCA
GACAGGTTTCAGAGATGGTTGGATCTCTGAAATTGTAAAATGAAGTATAA
TCTTAGGCTAAGGGAAGGATGCGTGTGAAGCTCTGGAGGTTGGTATAGTA
ATAGCTGACCTATTACTGCATTTGGGAGGGATCTGTCATAGCTTCCTTGC
CTCTTAATTAAGGGTGGTGTTTTTTTCTTTTATAAAAGAAGAATAAAAAG
ACTAATAAAAGTA
human SNHG12 deletion domain 2
(SEQ ID NO: 5)
CTTTCTCCCCGCCGCATTCCCGGTGTCGACTTACTAGCTGCAAGCCTCTG
CCTGCCTTCCTGCGCGCCGTTCCCCGCTAGTCGCTGCTGCTGGCGCGCAC
TCGCCGGGTTTTTCCTCCCACGGCCTCGAGCACTGGTCTTGGTGGTCGTA
AAAGGAGGAAAAGTAATAGTGAAGCTGGCCTAAATGTTGTAATCTGGTAT
ATGGCATGTGGGCTAGTTTCAGACAGGTTTCAGAGATGGTTGGATCTCTG
AAATTGTAAAATGAAGTATAATCTTAGGCTAAGGGAAGGATGCGTGTGAA
GCTCTGGAGGTTGGTATAGTAATAGCTGACCTATTACTGCATTTGGGAGG
GATCTGTCATAGCTTCCTTGCCTCTTAATTAAGGGTGGTGTTTTTTTCTT
TTAGATTCATGTTACATGTAAAGCTGTCCTCATTTGTGACTATGGACCTA
TGGAGTTGGGACAATCTCTATGGGAAGCAGAAGGCAAGGACCCCGGTCAT
TTTAGGTAGAAACAACAGCATGCTAATGCAAAAAATTATGCAGTGTGCTA
CTGAACTTCAGAGGTGATCAATAAAAGAAGAATAAAAAGACTAATAAAAG
TA
human SNHG12 deletion domain 3
(SEQ ID NO: 6)
CTTTCTCCCCGCCGCATTCCCGGTGTCGACTTACTAGCTGCAAGCCTCTG
CCTGCCTTCCTGCGCGCCGTTCCCCGCTAGTCGCTGCTGCTGGCGCGCAC
TCGCCGGGTTTTTCCTCCCACGGCCTCGAGATGGTGGTGAATGTGGCACG
GAGGAGCCGGGCCTTCCAACCCGGTGGGCCCGAGCTCCGAAAGGCCCCCT
CGGCAGTGAGAGGGGCGGGAGCCCGCGGGGGCCGCGCCCTTCTCTCGCTT
CGGACTGCGCAACGCTGCGCTCTGGGCTGACAGGCGGATAAAACGGTCCC
ATCAAGACTGAGAAAAAGCACACCAGCTATTGGCACAGCGTGGGCAGTGG
GGCCTACAGGATGACTGACTTAGTCTACAGAGATCCCGGCGTACTTAAGC
AGATGAAGACTCTTAAGATGACAGAAGGTGATTTTTCTGGTGATCGAGGA
CTTCCGGGGTAATGACAGTGATGAAATGCAGGGGACCTGGTTGCCCCCAA
GTTTCCTGGCAGTGTGTGATACTGAGGAGGTGAGCTTGTTTCTGGAGCTG
TGCTTTAAGGTAAAGTTGATCAGCTTAATCCTCCTGATCCCTTTCCCATC
GGATCTGAACACTGGTCTTGGTGGTCGTAAAAGGAGGAAAAGTGGCTAAG
GGAAGGATGCGTGTGAAGCTCTGGAGGTTGGTATAGTAATAGCTGACCTA
TTACTGCATTTGGGAGGGATCTGTCATAGCTTCCTTGCCTCTTAATTAAG
GGTGGTGTTTTTTTCTTTTAGATTCATGTTACATGTAAAGCTGTCCTCAT
TTGTGACTATGGACCTATGGAGTTGGGACAATCTCTATGGGAAGCAGAAG
GCAAGGACCCCGGTCATTTTAGGTAGAAACAACAGCATGCTAATGCAAAA
AATTATGCAGTGTGCTACTGAACTTCAGAGGTGATCAATAAAAGAAGAAT
AAAAAGACTAATAAAAGTA
human SNHG12 deletion domain 4
(SEQ ID NO: 7)
TGCAAGCCTCTGCCTGCCTTCCTGCGCGCCGTTCCCCGCTAGTCGCTGCT
GCTGGCGCGCACTCGCCGGGTTTTTCCTCCCACGGCCTCGAGATGGTGGT
GAATGTGGCACGGAGGAGCCGGGCCTTCCAACCCGGTGGGCCCGAGCTCC
GAAAGGCCCCCTCGGCAGTGAGAGGGGCGGGAGCCCGCGGGGGCCGCGCC
CTTCTCTCGCTTCGGACTGCGCAACGCTGCGCTCTGGGCTGACAGGCGGA
TAAAACGGTCCCATCAAGACTGAGAAAAAGCACACCAGCTATTGGCACAG
CGTGGGCAGTGGGGCCTACAGGATGACTGACTTAGTCTACAGAGATCCCG
GCGTACTTAAGCAGATGAAGACTCTTAAGATGACAGAAGGTGATTTTTCT
GGTGATCGAGGACTTCCGGGGTAATGACAGTGATGAAATGCAGGGGACCT
GGTTGCCCCCAAGTTTCCTGGCAGTGTGTGATACTGAGGAGGTGAGCTTG
TTTCTGGAGCTGTGCTTTAAGGTAAAGTTGATCAGCTTAATCCTCCTGAT
CCCTTTCCCA

### Statistics

Data throughout the paper are expressed as mean±SD. Statistical differences between two groups was assessed by unpaired two-tailed Student's t test and for more than two groups, one-way ANOVA analysis was used. A probability of p<0.05 was considered statistically significant. Ns, not significant; * p<0.05; ** p<0.01; *** p<0.001; ****p<0.0001. For illustration of differentially expressed genes GraphPad software (V.7.0a or 8) was used.

TABLE 4
Primer List

| | SEQ | |
|---|---|---|
| | ID | |
| Oligonucleotides | NO: | Source |
| Mouse: b-actin Forward | 8 | PrimerBank |
| GAAATCGTGCGTGACATCAAAG | | |
| Mouse: b-actin Reverse | 9 | PrimerBank |
| TGTAGTTTCATGGATGCCACAG | | |
| Mouse: GAPDH Forward | 10 | PrimerBank |
| AGGTCGGTGTGAACGGATTTG | | |
| Mouse: GAPDH Reverse | 11 | PrimerBank |
| TGTAGACCATGTAGTTGAGGTCA | | |
| Mouse: SNHG12 Forward | 12 | |
| TCCAAGGAAGCACGGGTTAT | | |
| Mouse: SNHG12 Reverse | 13 | |
| TTCTGGTCTCCCTCCTCACA | | |
| Mouse: CDKN1A Forward | 14 | PrimerBank |
| CCTGGTGATGTCCGACCTG | | |
| Mouse: CDKN1A Reverse | 15 | PrimerBank |
| CCATGAGCGCATCGCAATC | | |
| Mouse: CDKN2A Forward | 16 | PrimerBank |
| CGCAGGTTCTTGGTCACTGT | | |
| Mouse: CDKN2A Reverse | 17 | PrimerBank |
| TGTTCACGAAAGCCAGAGCG | | |
| Mouse: CDKN1B Forward | 18 | PrimerBank |
| TCAAACGTGAGAGTGTCTAACG | | |
| Mouse: CDNK1B Reverse | 19 | PrimerBank |
| CCGGGCCGAAGAGATTTCTG | | |
| Human: HPRT Forward | 20 | |
| GCTATAAATTCTTTGCTGACCTGCTG | | |
| Human: HPRT Reverse | 21 | |
| AATTACTTTTATGTCCCCTGTTGACTGG | | |
| Human: CARMN Forward | 22 | |
| CAGGAATGTGGGACATGGAA | | |
| Human: CARMN Reverse | 23 | |
| GGTGTGGAGCCTGGATCTTT | | |
| Human: CDKN1A Forward | 24 | PrimerBank |
| TGTCCGTCAGAACCCATGC | | |
| Human: CDKN1A Reverse | 25 | PrimerBank |
| AAAGTCGAAGTTCCATCGCTC | | |
| Human: CDKN2A Forward | 26 | PrimerBank |
| GGGGGCACCAGAGGCAGT | | |
| Human CDKN2A Reverse | 27 | PrimerBank |
| GGTTGTGGCGGGGGCAGTT | | |
| Human CDKN1B Forward | 28 | PrimerBank |
| AACGTGCGAGTGTCTAACGG | | |
| Human CDKN1B Reverse | 29 | PrimerBank |
| CCCTCTAGGGGTTTGTGATTCT | | |
| Human: SNHG12 Forward | 30 | |
| CCTTGCCTCTTAATTAAGGGTGG | | |
| Human: SNHG12 Reverse | 31 | |
| CTTGCCTTCTGCTTCCCATAG | | |
| Human: TFIIB Forward | 32 | PrimerBank |
| ACCACCCCAATGGATGCAGACAG | | |
| Human: TFIIB Reverse | 33 | PrimerBank |
| ACGGGCTAAGCGTCTGGCAC | | |
| Human: DNA-PK Forward | 34 | PrimerBa nk |
| CCTGGCCGGTCATCAACTG | | |
| Human: DNA-PK Reverse | 35 | PrimerBank |
| AGTAAGGTGCGATCTTCTGGC | | |
| Human: Malat1 Forward | 36 | |
| GGATTCCAGGAAGGAGCGAG | | |
| Human: Malat1 Reverse | 37 | |
| ATTGCCGACCTCACGGATTT | | |
| Human: Snora16 Forward | 38 | |
| ATAGCTGCTGTGGTCAAAAAGG | | |
| Human: Snora16 Reverse | 39 | |
| TTGCAGATCAGTTACAACAAACAGA | | |
| Human: Snora44 Forward | 40 | |
| AAGGGCTGTGGCTGGTCATA | | |
| Human: Snora44 Reverse | 41 | |
| GCAGCTTGCAGGTATTGTACTGA | | |
| Human: Snora16 Probe | 42 | |
| TGACAGTTTTCCTTGACGGTCGCC | | |
| Human: Snora44 Probe | 43 | |
| CCATGGGATCTCCAACTGCATGCA | | |
| Human: TRNAU1AP Forward | 44 | |
| CCAGAACACAGGCAGCTACAG | | |
| Human: TRNAU1AP Reverse | 45 | |
| TCTTCCAATGCATCATCTCCA | | |
| Sus Scrofa: GAPDH Forward | 46 | |
| CCCCTTCATTGACCTCCACT | | |
| Sus Scrofa: GAPDH Reverse | 47 | |
| TTGACTGTGCCGTGGAACTT | | |
| SuS Scrofa: SNHG12 Forward | 48 | |
| TGACCTTTCCAGTGAGGGAAG | | |
| Sus Scrofa: SNHG12 Reverse | 49 | |
| GCTCCAGGAAAAGCTGGTGT | | |

### Example 1: Identification of Dynamically Regulated lncRNAs in Atherosclerosis and In Vivo Loss- and Gain-of-Function of the Conserved lncRNA Small Nucleolar Host Gene 12 (SNHG12)

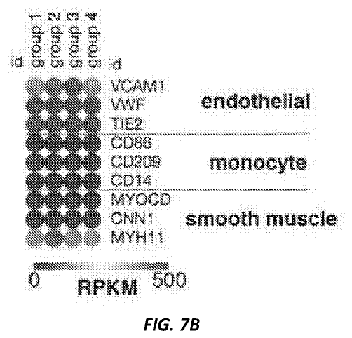

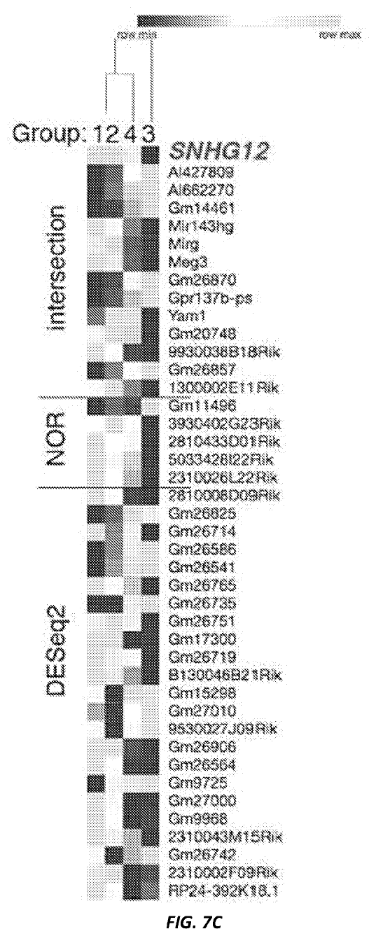

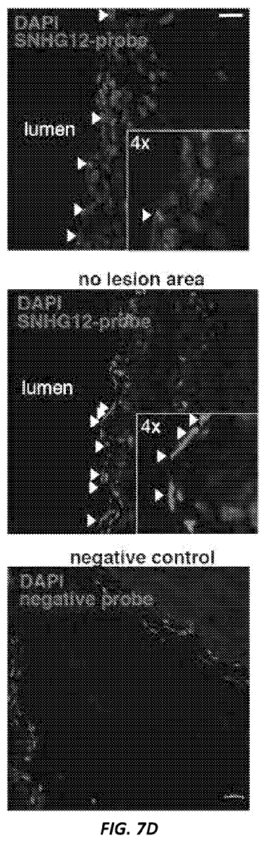

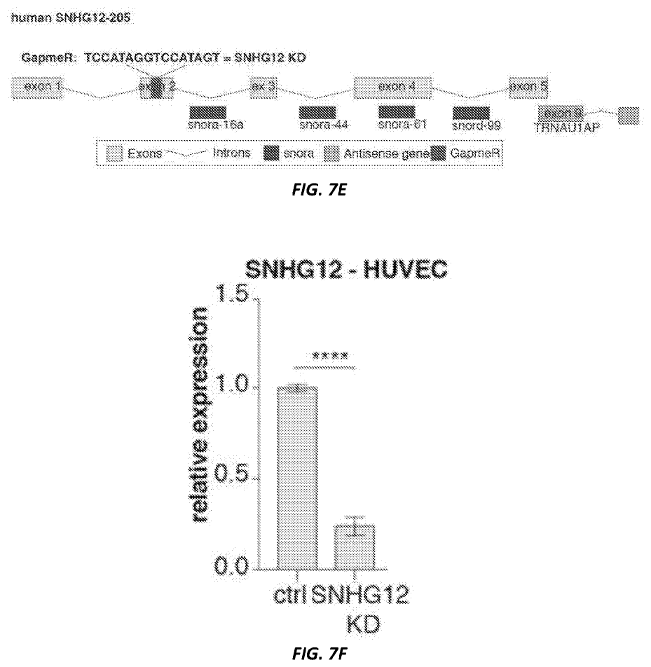

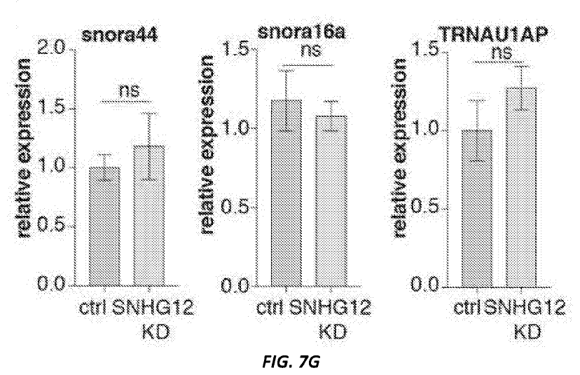

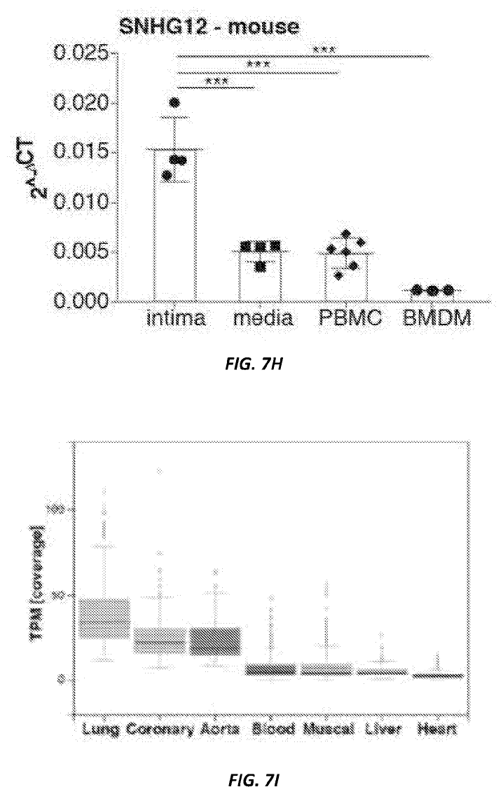

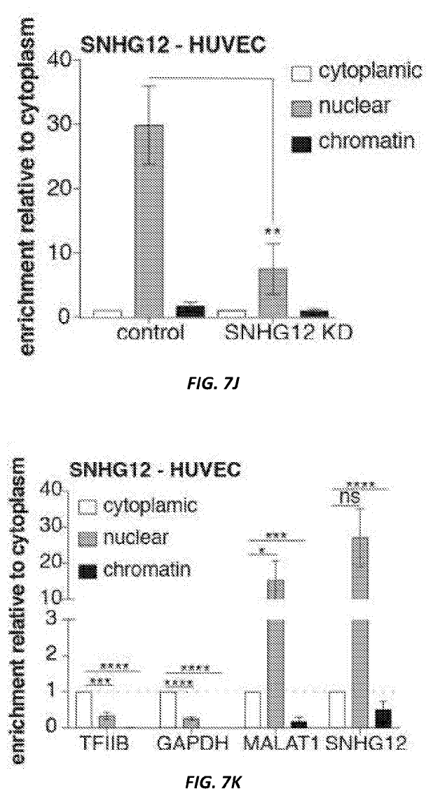

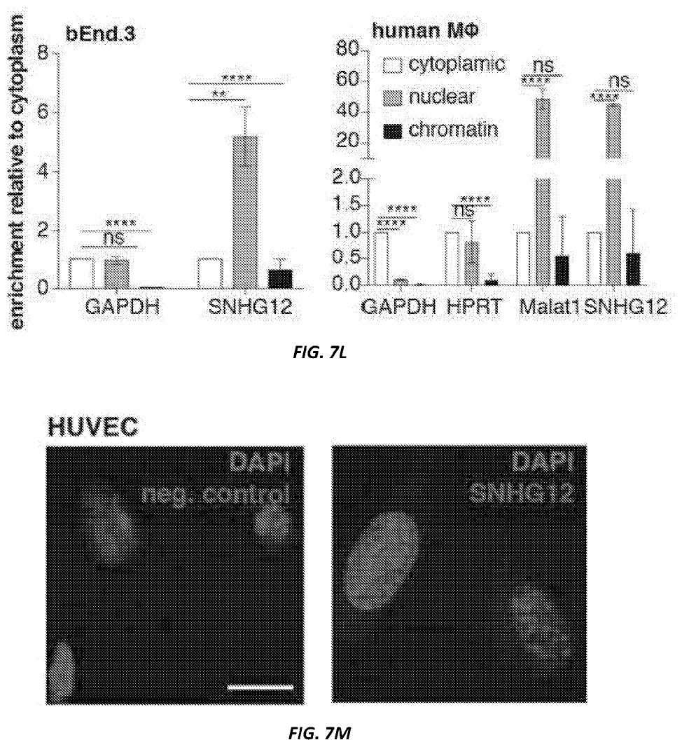

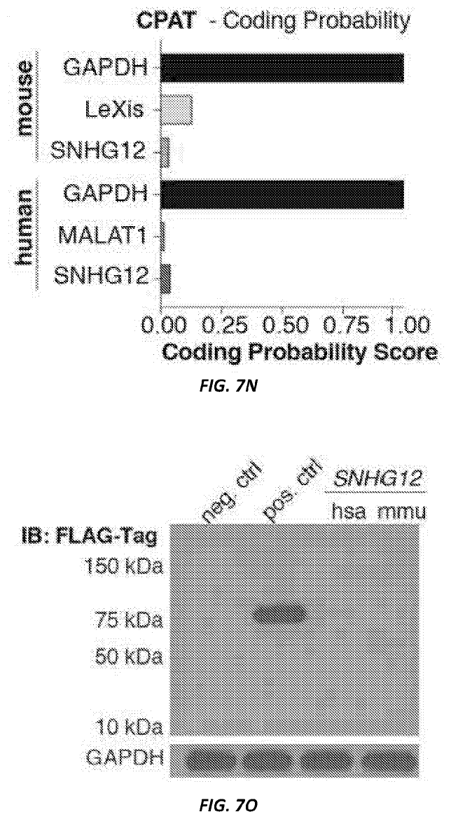

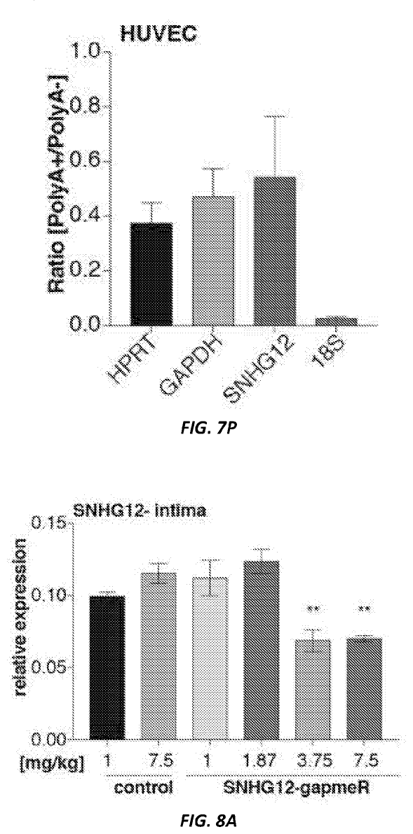

To identify lncRNAs whose expression changes in the aortic intima during the progression and regression phases of atherosclerosis, LDLR^{-/-} mice consumed a high cholesterol diet (HCD) and RNA derived from the aortic intima after 0, 2, and 12 weeks on HCD (progression phase; groups 1-3) and at 18 weeks after 6 weeks of resumption of a normal chow diet (regression phase; group 4) (FIG. 1A), was used for RNA-Seq profiling to capture differentially expressed lncRNAs (log 2-fold change (1.5); FDR<0.05) compared to group 1 (FIG. 1B). Changes in lesional macrophages in the aortic sinus verified progression and regression of atherosclerosis and cell-type specific markers established the purity of the endothelial enriched tunica intima RNA (FIGS. 7A, 7B). Due to potential miscalculations using DESeq2 for two transcripts on opposite strands, an algorithm to exclude all reads with an antisense transcript was also applied (i.e. No Overlapping Reads (NOR)). This approach identified 37 lncRNA transcripts from DESeq2 and 19 lncRNAs using NOR, whereas 14 lncRNAs were commonly dysregulated in both algorithms (FIG. 1C, FIG. 7C). Among these transcripts, we noted a highly conserved and most abundantly expressed lncRNA named SNHG12, which is expressed from the syntenic location in the mouse, human, and pig genomes; sequences were verified by 5′-RACE-PCR (FIG. 1D). RNA-Seq results for SNHG12 were further verified by RT-qPCR and by RNA in situ hybridization (ISH), showing reduced expression of SNHG12 in the aortic intima after 12 weeks of HCD (group 3) and near normalization during lesion regression (group 4) (FIG. 1E, FIG. 7D). Although SNHG12 contains intronic cryptically encoded small RNA and overlaps partially with TRNAU1AP (i.e. 9 bp), gapmeR-mediated silencing of SNHG12 did not affect the expression of either the small RNAs or TRNAU1AP (FIG. 7E-G). SNHG12 expression in the vascular endothelium exceeds that in the aortic media, peripheral blood mononuclear cells (PBMCs), and bone marrow-derived mononuclear cells (BMDMs) (FIG. 7H). In addition, analysis from genotype-tissue expression (GTEx) revealed that cardiovascular or endothelial-enriched tissues express SNHG12 (FIG. 7I). SNHG12 is a nuclear expressed lncRNA in human and mouse ECs and primary macrophages (FIGS. 7J-7M), does not encode short peptides (FIGS. 7N, 7O), and is polyadenylated (FIG. 7P).

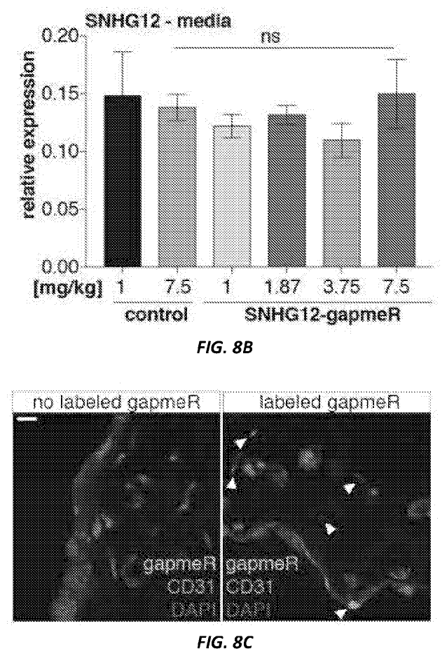

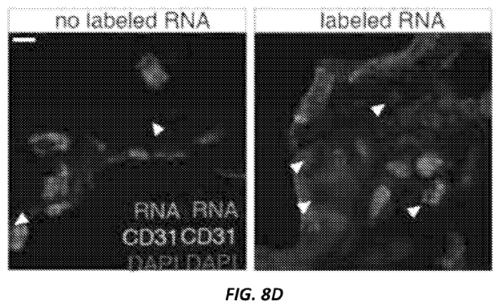

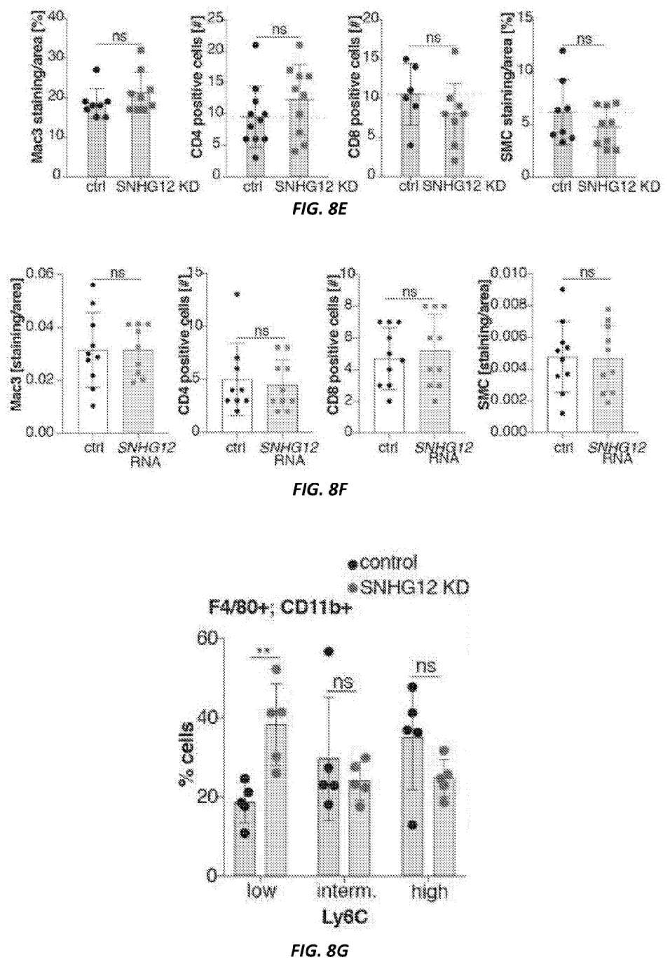

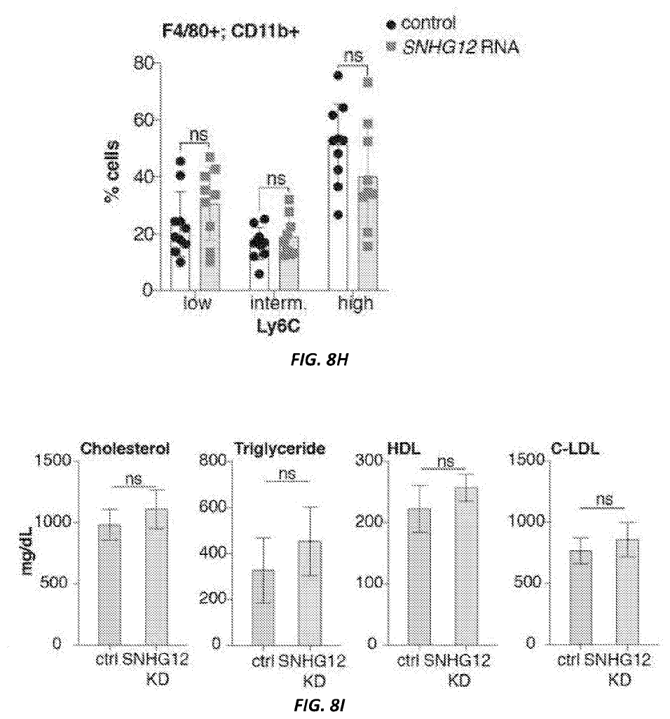

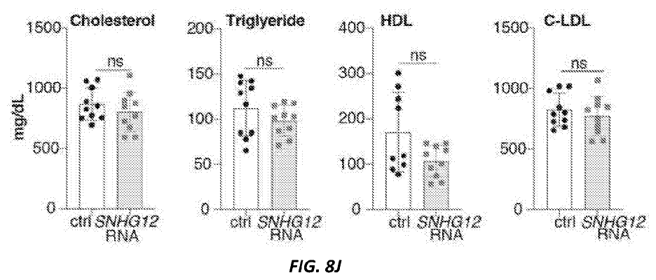

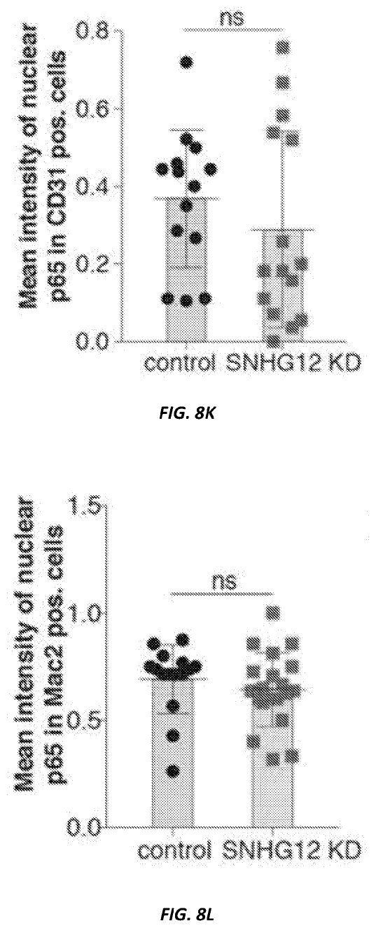

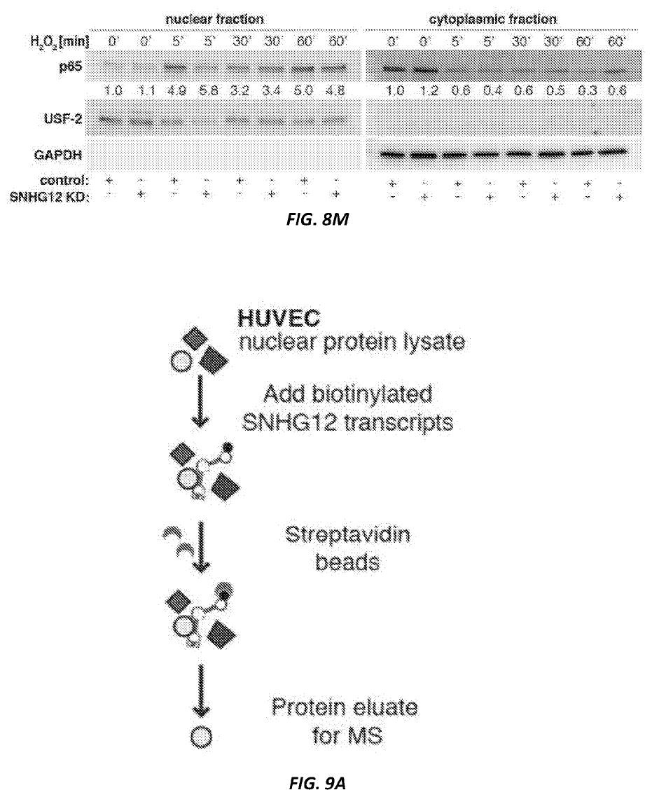

To explore the role of systemically delivered SNHG12-gapmeRs in atherosclerosis, LDLR^{-/-} mice received vehicle control or SNHG12-gapmeR (7.5 mg/kg/2×weekly i.v.) for 12 weeks on HCD (FIG. 1F). After 12 weeks on HCD, gapmeR-mediated silencing of SNHG12 reduced its expression in the aortic intima by 40% and 32% in PBMCs, but not in the aortic media (FIG. 1G, 8A-C). Analysis of atherosclerotic lesion formation by Oil-Red O (ORO) staining revealed a 2.4-fold increase in lesion area in the aortic sinus and 1.7-fold increase in the descending thoracoabdominal aorta compared to negative control (FIGS. 1H, 1I). To explore the effects of SNHG12 overexpression on atherosclerotic progression, we delivered intravenously a 5′-capped, and 2′-O-methylated SNHG12 RNA transcript to ApoE^{-/-} mice placed on HCD for 6 weeks (n=10/group/15 μg/2×weekly). This intervention achieved a 4-fold increase of SNHG12 expression in the aortic intima (FIGS. 1J, 1K, FIG. 8D) and reduced lesion areas in the aortic sinus and descending aorta by 34% and 40%, respectively (FIGS. 1L, 1M).

Surprisingly, neither loss-nor gain-of-function of SNHG12 changed the accumulation of lesional cells bearing markers of macrophages or vascular smooth muscle cells, or of CD4⁺ or CD8⁺ T cells, (FIGS. 8E, 8F). SNHG12-gapmeRs also reduced SNHG12 expression in PBMCs, but did not alter monocyte polarization as gauged by Ly6C expression monitored by FACS (FIGS. 8G, 8H). Neither loss- or gain-of-function of SNHG12 altered blood cholesterol, triglyceride, HDL or c-LDL) (FIGS. 8I, 8J). Activation of the vascular endothelium depends in large part on pro-inflammatory processes mediated primarily through NF-κB-regulated signaling pathways (2). SNHG12 knockdown in LDLR^{-/-} mice did not affect nuclear NF-κB p65 detected by immunofluorescence (IF) in CD31⁺ECs and Mac2⁺ macrophages of aortic arch sections (FIG. 8K,L). Nor did SNHG12-gapmeRs promote p65 nuclear translocation in human umbilical vein endothelial cells (HUVECs) treated with H₂O₂ (FIG. 8M). Remarkably, these findings indicated that reducing SNHG12 expression in the aortic intima promotes atherosclerotic lesion formation in LDLR^{-/-}, whereas delivery of SNHG12 decreased plaque burden. Furthermore, these effects in both studies appeared to be independent of circulating lipid profile or of lesional leukocyte accumulation.

### Example 2: LncRNA SNHG12 Interacts with DNA-Dependent Protein Kinase (DNA-PK)

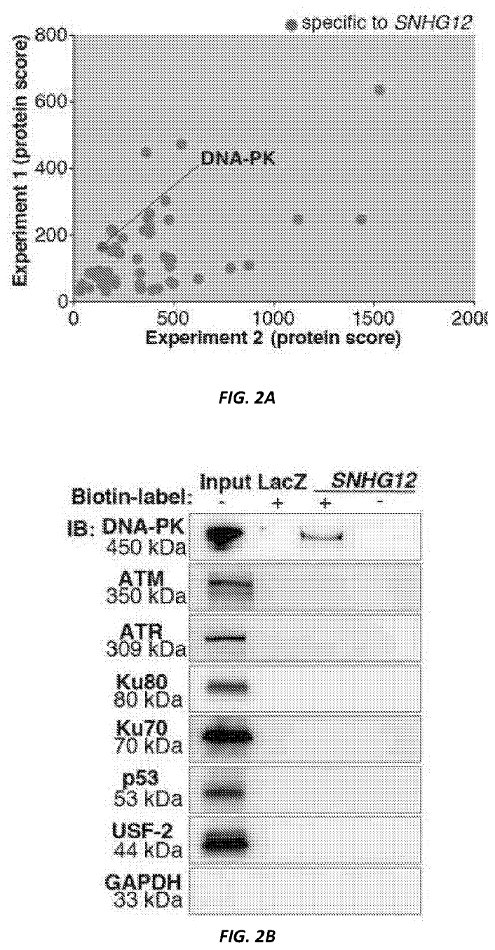

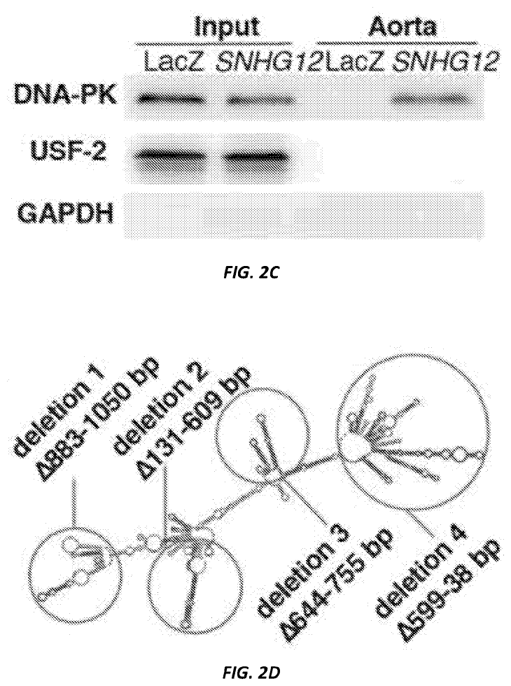

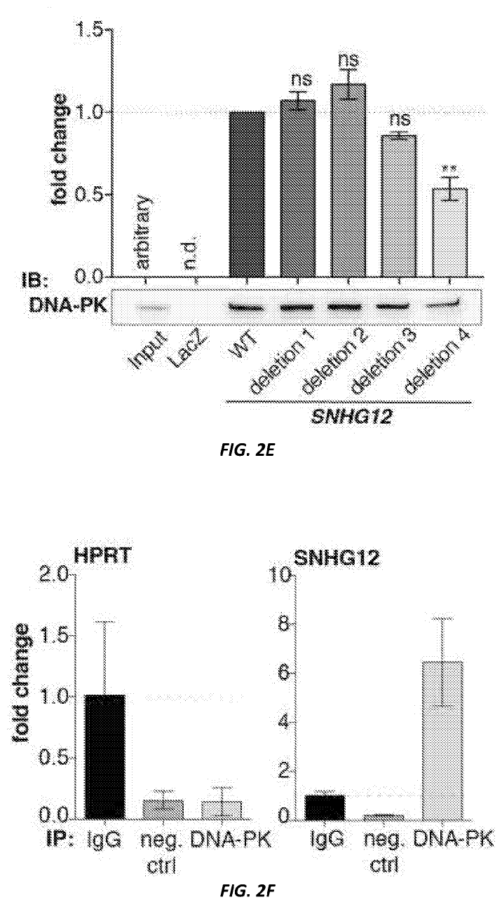

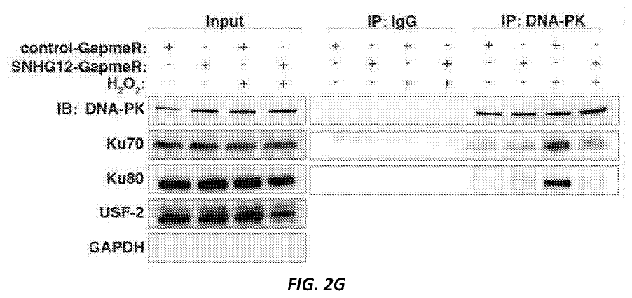

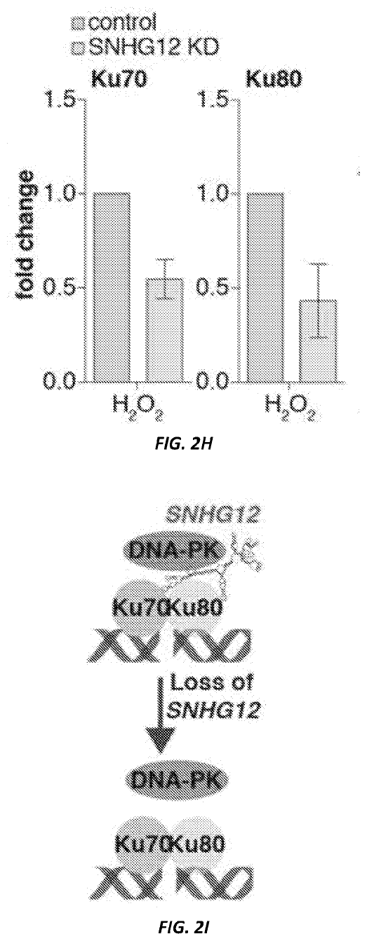

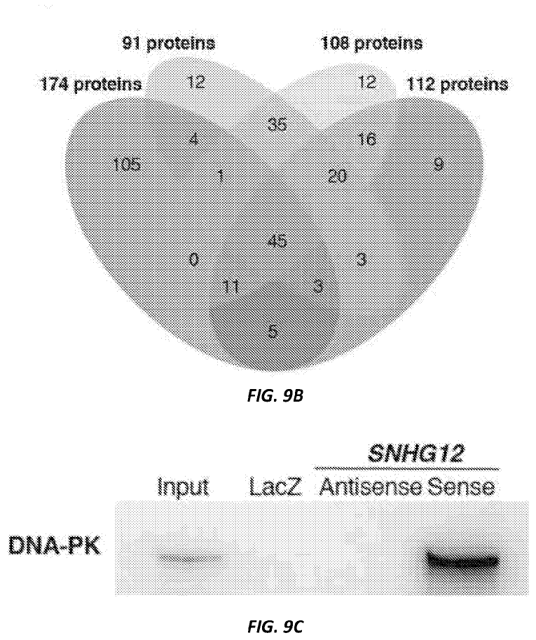

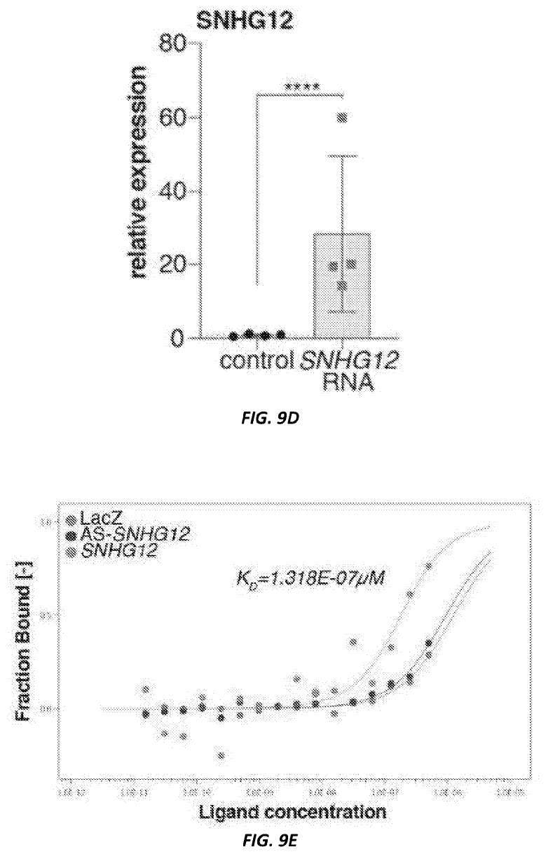

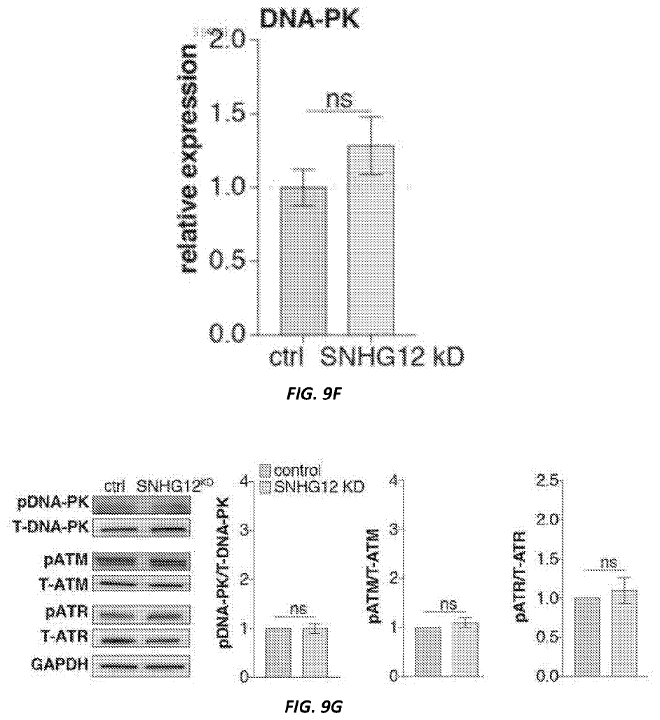

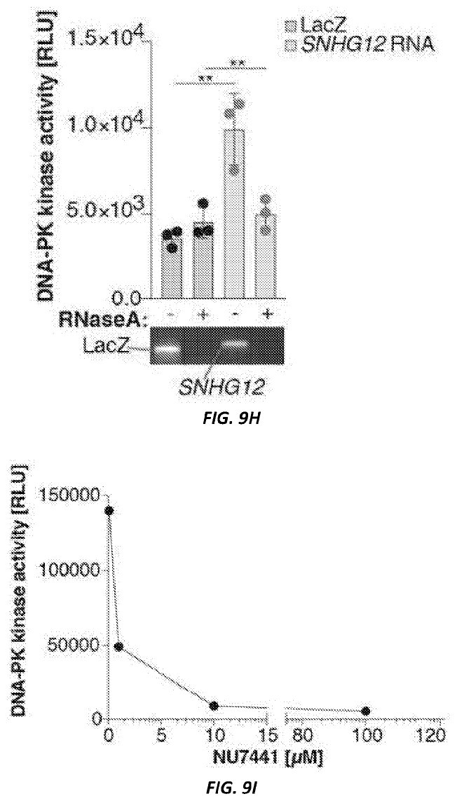

To identify potential SNHG12-interacting proteins that may inform mechanisms underlying these findings, biotin-labeled T7 in vitro transcribed SNHG12 or LacZ was incubated with nuclear protein lysate of HUVECs (FIG. 9A). Peptides that specifically bound to biotin-labeled SNHG12 transcript were identified by liquid chromatography-mass spectrometry (LC-MS/MS) analysis. This unbiased approach led to the identification of DNA-PK, an important sensor and mediator in the DNA damage response (DDR) and DNA repair process of non-homologues end joining (NHJ) (18, 19) (FIG. 2A, FIG. 9B). DNA-PK was only detectable in the eluate of biotin-labeled SNHG12 compared to the LacZ, antisense transcript of SNHG12 or unlabeled SNHG12, whereas other major regulators of the DDR (ATM, ATR and p53) were not found (FIG. 2B, FIG. 9C). This interaction could be validated in vivo by i.v. injection of biotin-labeled SNHG12 compared to LacZ. Remarkably, DNA-PK was recovered in aortic protein lysate of biotin-labeled SNHG12 injected C57B1/6 mice (n=4/group) (FIG. 2C). Successful delivery of labeled SNHG12 was verified by RT-qPCR (FIG. 9D). Furthermore, biophysical studies determined the equilibrium dissociation constant (KD) for the SNHG12-DNA-PK interaction (1.318E-07 μM) (FIG. 9E). To map more precisely the interaction of SNHG12-DNA-PK, predicted domains of the secondary structure of SNHG12 were deleted for subsequent lncRNA pulldown experiments (FIG. 2D). Deletion of domain-4 reduced DNA-PK binding by ˜50%, suggesting that this domain participates in binding to DNA-PK (FIG. 2E). Conversely, SNHG12 expression rose 6-fold in RNA isolated after DNA-PK immunoprecipitation (IP) compared to IgG control; this effect was specific to SNHG12, compared to other transcripts such as HPRT (FIG. 2F). This interaction did not involve transcriptional regulation, since SNHG12 silencing did not affect DNA-PK expression in HUVECs or phosphorylation of pDNA-PK, pA™, or pATR (FIGS. 9G, 9F).

To identify the functional consequences of the interaction of SNHG12 with DNA-PK, we assessed the effect of SNHG12 on DNA-PK's kinase activity (DNA-PKcs). Incubation of purified DNA-PK protein with in vitro transcribed SNHG12 assessed the ability of DNA-PKcs to convert ATP to ADP. In the presence of SNHG12, DNA-PKcs increased by 2-fold compared to the LacZ control and positive control NU7441 (FIG. S3H, I) (20). RNAseA treatment reduced this DNA-PKcs activity, suggesting that SNHG12 facilitates DNA-PKcs. DNA-PKcs recruitment to and activation by DNA requires the Ku complex, a heterodimer containing two subunits of 70 and 80 kDa, that binds to DNA double-strand breaks (DSBs) (18). The ability of DNA-PK to bind Ku70/80 was assessed under ROS-induced DNA damage conditions by performing DNA-PK IP and subsequent immunoblotting for Ku70 and Ku80. In HUVECs transfected with SNHG12-gapmeRs, the expression of Ku70 and Ku80 fell significantly (P=0.0016 and 0.0218, respectively) under H₂O₂-induced DDR compared to control-gapmeRs following DNA-PK IP (FIG. 2G, H). Taken together, these findings indicated that DNA-PK can actively bind to lncRNA SNHG12, which in turn facilitated the ability of DNA-PKcs to bind to Ku70/80, an important mediator of the DDR (FIG. 2I).

### Example 3: SNHG12 Regulates DSBs, Leading to Increased DNA Damage in ECs and Macrophages

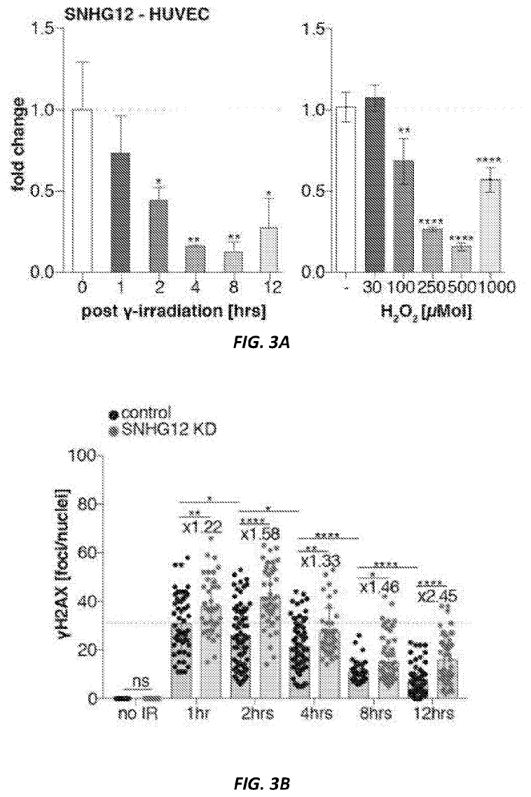

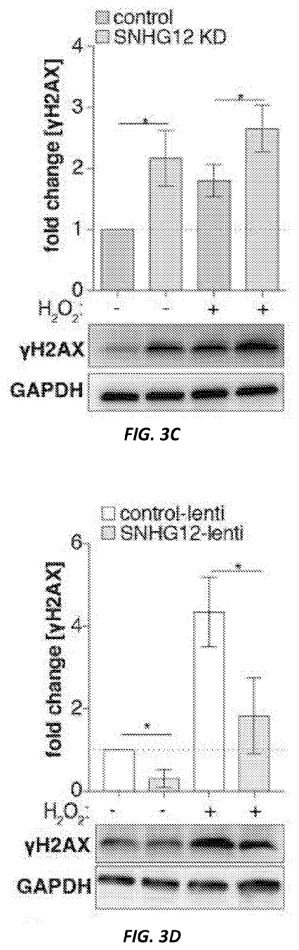

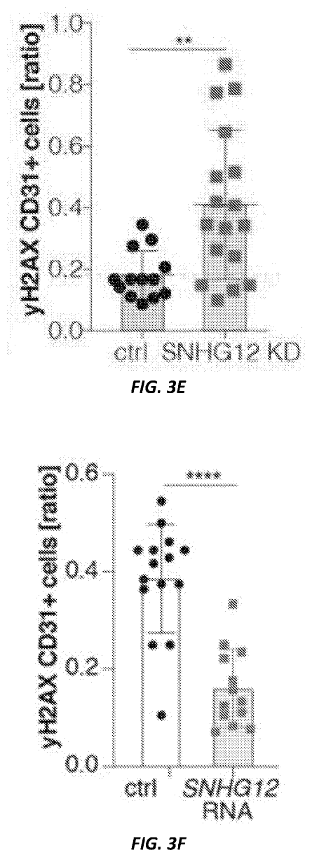

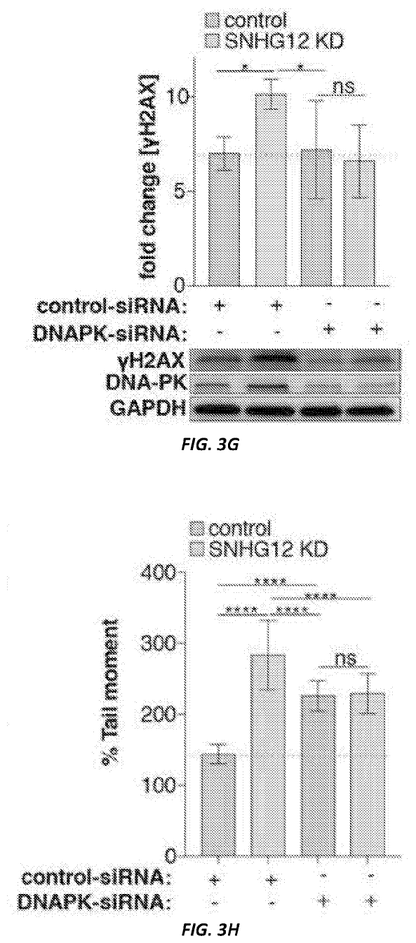

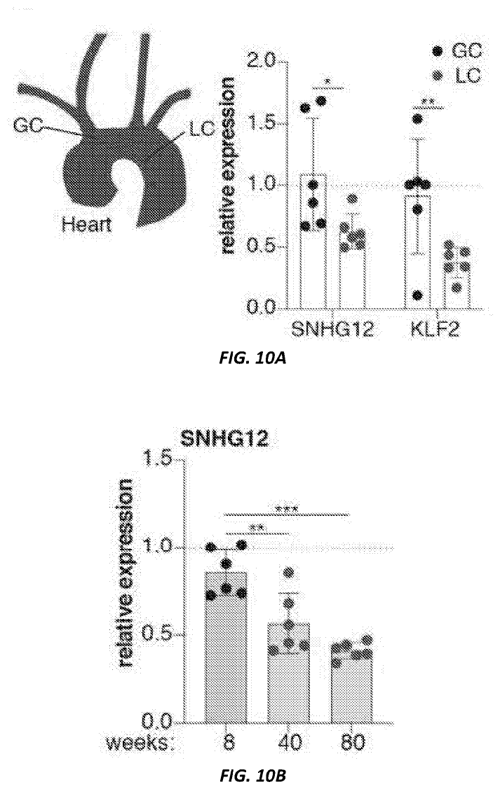

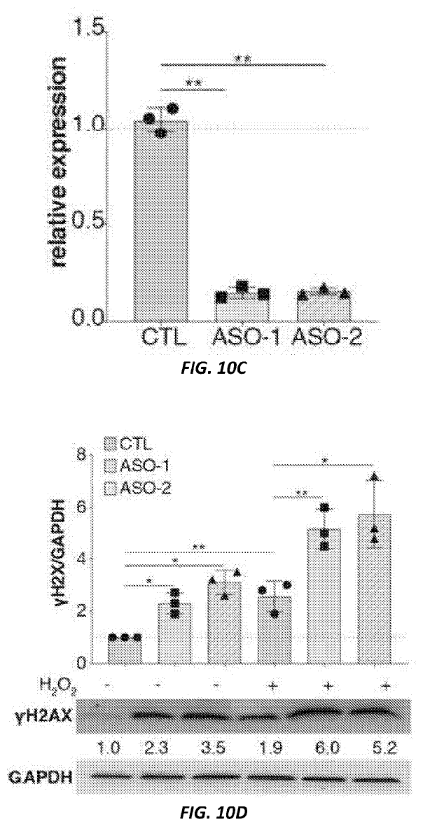

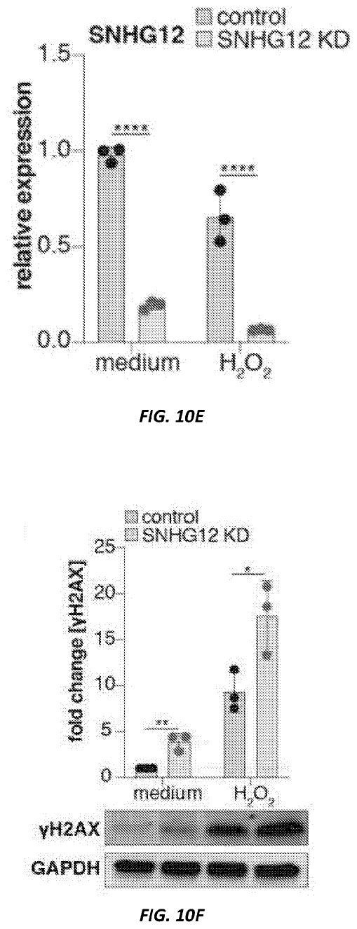

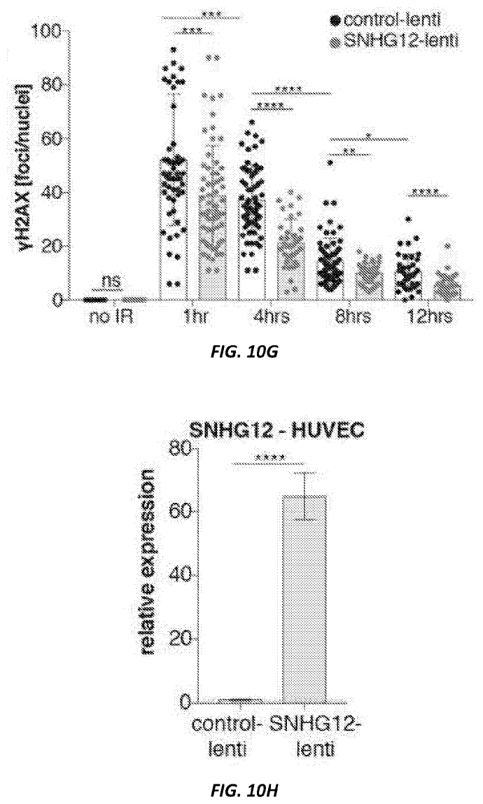

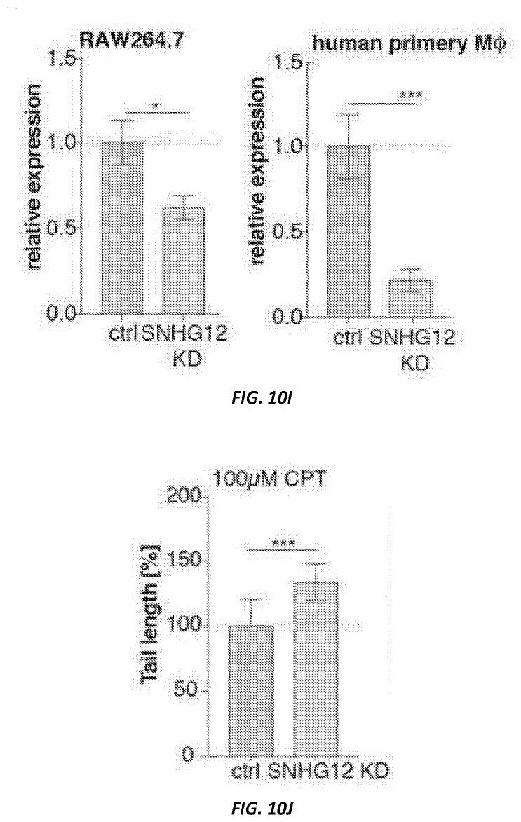

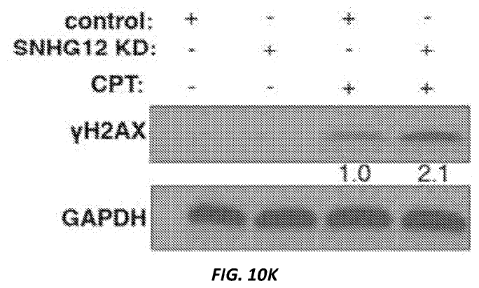

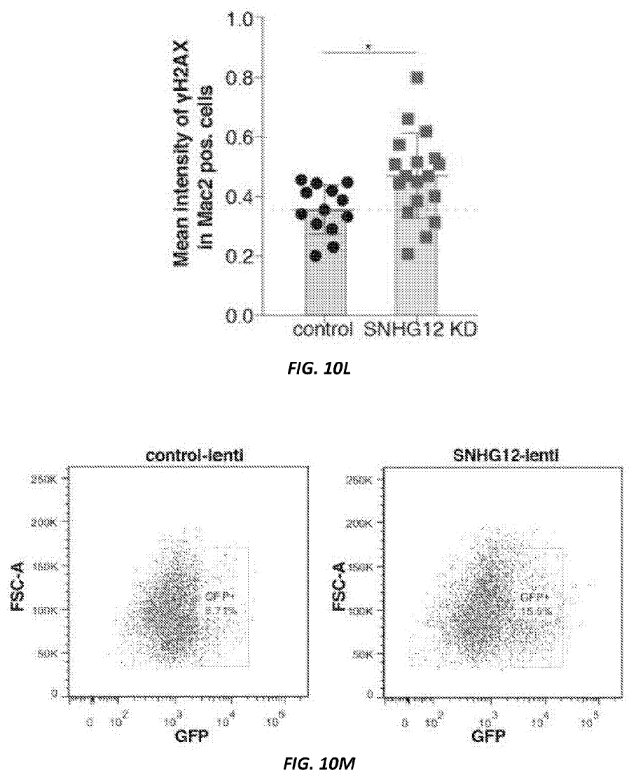

The DDR involves DNA strand break recognition, followed by the initiation of a cascade that promotes DNA repair. In response to extrinsic stress, such as γ-irradiation, or intrinsic stimuli such as H₂O₂, SNHG12 expression fell dose-dependently (FIG. 3A). Other stress triggers such as shear stress and aging significantly (P=0.0392 and 0.0420, respectively) reduce SNHG12 expression (FIGS. 10A, 10B). Phenotypic consequences of SNHG12 on the DDR was tested in ECs treated with intrinsic (γ-irradiated) and extrinsic (H₂O₂) triggers of DSBs. ECs transfected with SNHG12-gapmeRs showed increased nuclear γH2AX foci across all time points with the most pronounced effect occurring after 12 hrs by 2.5-fold (FIG. 3B). By Western blot analysis, γH2AX increased in ECs by 2.2-fold and 1.4-fold under basal and H₂O₂-induced DNA damage conditions, respectively (FIG. 3C). This effect of SNHG12 on the DDR was recapitulated in mouse ECs using two additional antisense oligonucleotides (ASO) for knockdown (FIGS. 10C, 10D) as well as on human arterial endothelial cells (HAECs) (FIGS. 10E, 10F). Conversely, lentiviral overexpression of SNHG12 reduced γH2AX foci formation by 50% as early as 1 hr post-γ-irradiation and reduced H₂O₂-induced γH2AX phosphorylation by 2-fold as shown by Western analysis (FIGS. 3D, 10G, 10H). While ECs express more SNHG12 compared to leukocytes, SNHG12-mediated effects on the DDR in leukocytes was assessed in human primary macrophages and mouse RAW264.7 cells using camptothecin (CPT), a topoisomerase inhibitor that induces DSBs (21). DNA Comet assay to quantify individual cell DNA damage showed that SNHG12 knockdown prolonged DNA-tail length in the presence of CPT by 45%, and increased γH2AX phosphorylation by 2-fold as analyzed by Western (FIGS. 10I-10K).

Consistent with these in vitro findings, lesions of LDLR^{-/-} mice treated with SNHG12-gapmeRs exhibited a 2-fold increase in γH2AX foci in the vascular endothelium (CD31⁺ cells) (FIG. 3E). Furthermore, γH2AX also rose significantly (P=0.0157) by 1.4-fold in lesional macrophages (Mac2⁺ cells) (FIG. 10L). Moreover, delivery of SNHG12 to the vessel wall reduced γH2AX foci in ApoE^{-/-} mice after 6 weeks on HCD (FIG. 3F).

To test whether the SNHG12-mediated effect on γH2AX foci formation depends on DNA-PK, DNA-PK was silenced in combination with simultaneous SNHG12 knockdown. DNA-PK silencing blocked the SNHG12-gapmeR mediated increase of γH2AX foci and tail moment after ROS- or CPT-induced DNA damage (FIGS. 3G, 3H). Further, since DNA-PK is implicated in regulating NHEJ (3) and SNHG12 is dependent on DNA-PK, HUVECs were stably transduced with a DNA repair reporter (pDRR). I-SceI cuts the integrated pDRR to generate DSBs; repair through MET leads to expression of GFP (22). Indeed, lentiviral overexpression of SNHG12 promoted NHEJ as indicated by a 2.5-fold increase in GFP positive cells compared to control (FIG. 3I, FIG. 10M). These findings support the role of SNHG12 in the DDR in a DNA-PK-dependent manner and show that this lncRNA can prevent DNA damage accumulation in atherosclerotic lesions.

### Example 4: Phenotypic Consequences of Accumulating DNA Damage

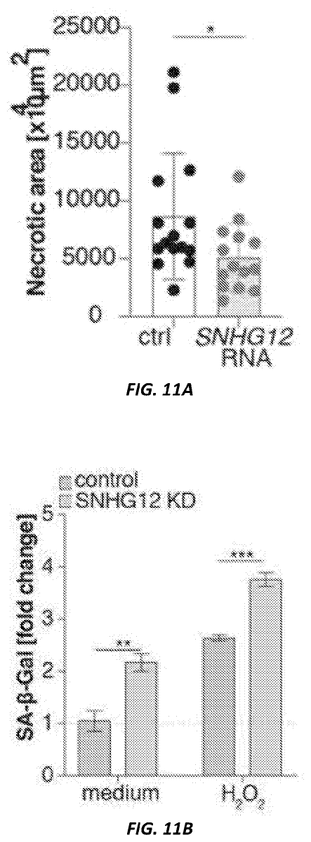

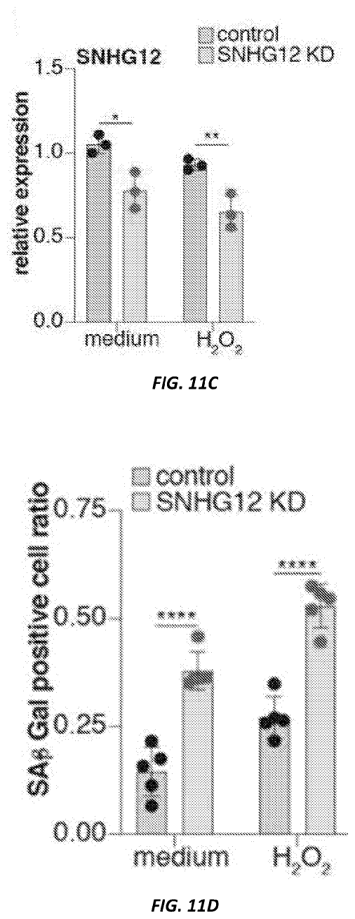

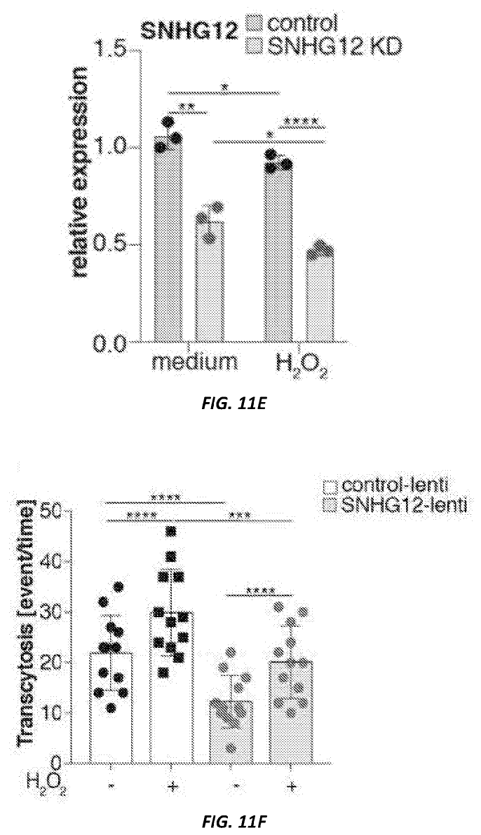

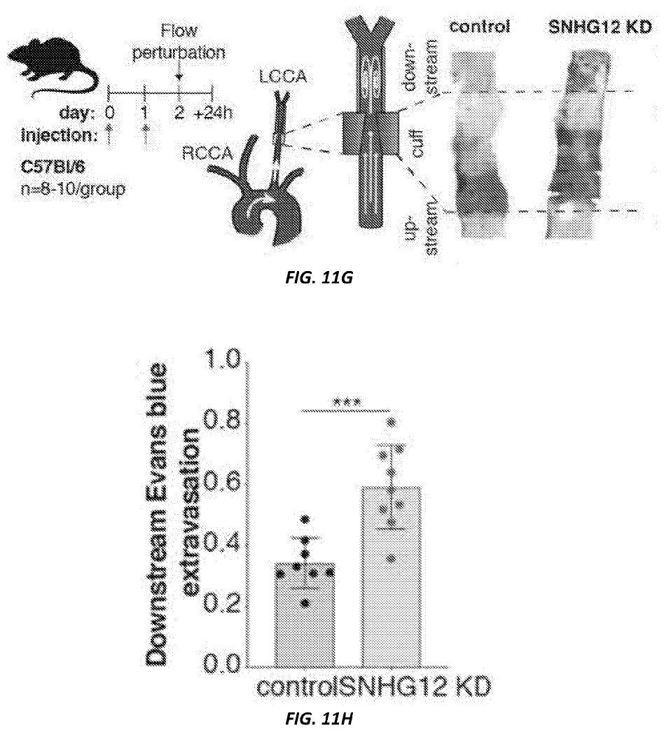

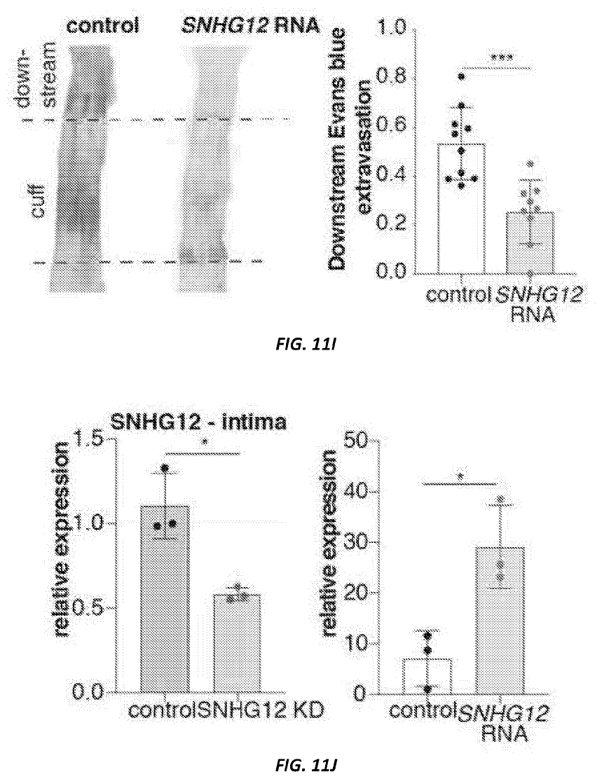

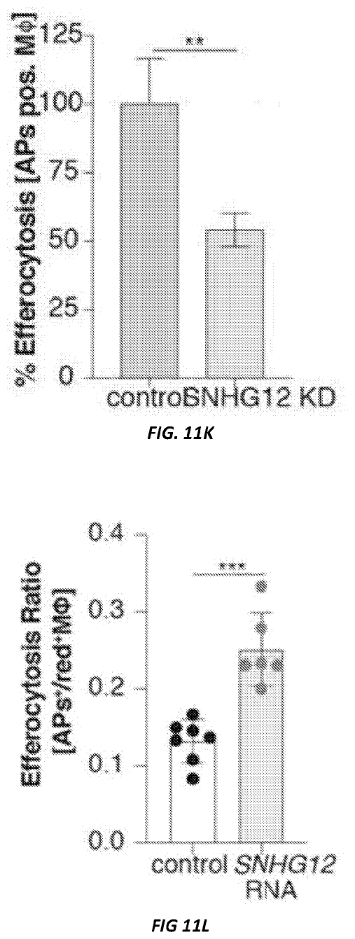

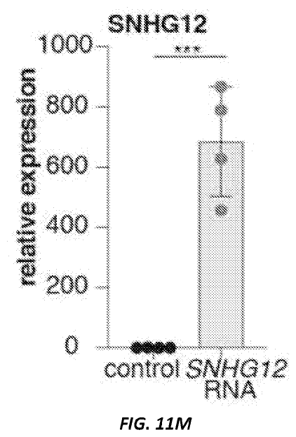

Exploration of the pathways and processes affected by SNHG12 knockdown used unbiased genome-wide RNA-Seq on SNHG12-gapmeR transfected ECs exposed to H₂O₂. This undertaking identified a number of dynamically regulated genes (log 2FC<0.5, FDR<0.05) (FIG. 4A). Gene Set Enrichment Analysis (GSEA) revealed the p53 pathway and UV response among the top 10 processes affected (FIG. 4B). p53 was strongly predicted to be activated (norm. enrichment score=1.424, FDR q-value=0.0089) (FIG. 4C). To test this possibility in vitro, knockdown of SNHG12 significantly (P=0.0089) induced phosphorylation of p53 in response to reactive oxygen species (ROS) without altering total p53 (FIG. 4D). Furthermore, p53 binding affinity to DNA increased upon SNHG12 knockdown (FIG. 4E). Increased p53 activity to maintain genomic integrity is a well-established hallmark in the DDR (23) and the p53-p21 axis importantly regulates stress-induced senescence (24). Since inhibition of DNA-PK accelerates senescence (25), we evaluated the role of SNHG12 in this process. To this end, EC senescence was triggered with low dose of H₂O₂ (30 μM) for 1 hr, followed by another 3 days of culturing with normal growth medium. SNHG12 knockdown increased the expression for the senescence markers p16 (by 1.5-fold) and p21 (by 1.5-fold) (FIG. 4F). In lesions with reduced SNHG12 expression after delivery of gapmeRs in vivo, p16, p21, and p27 all rose by up to 2-fold in the aortic intima of LDLR^{-/-} mice (FIG. 4G). Furthermore, those lesions harbored more than 2-fold higher acellular areas than controls (FIG. 4H). Conversely, SNHG12 overexpression in the vessel wall reduced plaque necrosis (FIG. 11A). SNHG12 knockdown increased SA-βgal positive cells in HUVECs and HAECs, conversely overexpression of SNHG12 reduced SA-βgal (FIG. 11B-E, FIG. 4I).

As a consequence of increased DNA damage, ECs may exhibit impaired homeostatic control of functions important to lipoprotein entry and macrophages may display impaired clearance of cellular debris or apoptotic cells. To assess lipoprotein entry, we performed transcytosis assays using confluent monolayers of human coronary artery endothelial cells (HCAECs) and total internal reflection fluorescence microscopy (TIRF) (26). SNHG12 knockdown significantly (P<0.0001) increased DiI-LDL transport from the apical to the basolateral membrane by 1.8-fold (FIG. 4J). Conversely, lentiviral overexpression of SNHG12 rescued H₂O₂-induced EC permeability to LDL (FIG. 11F). To address in vivo permeability in arteries, a cone-shape polyethylene constrictive cuff (CC) was placed on the left common carotid artery (LCCA) (27). Flow perturbation to promote endothelial dysfunction was measured 24 hrs post-surgery. Downstream Evans Blue area of the CC was more elongated in longitudinal cross sections of the LCCA after inhibiting SNHG12 expression compared to the negative control (FIGS. 11G, 11H). Conversely, administration of SNHG12 RNA by i.v. injection reduced downstream Evans Blue extravasation (FIG. 11I). Efficiency of SNHG12 knockdown and delivery was assessed in the aortic intima by RT-qPCR analysis (FIG. 11J).

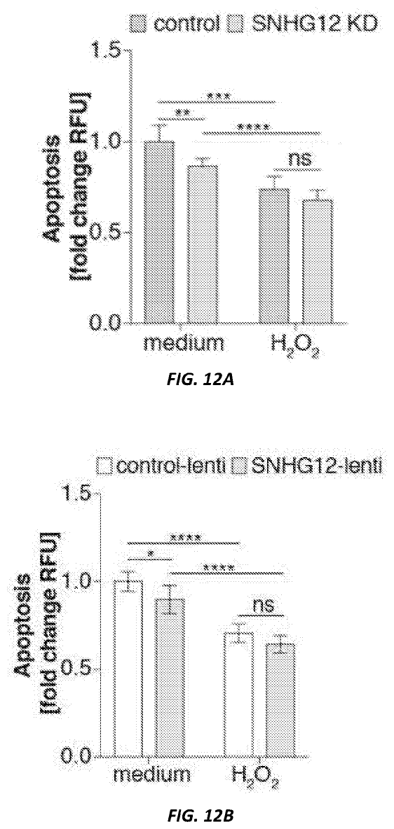

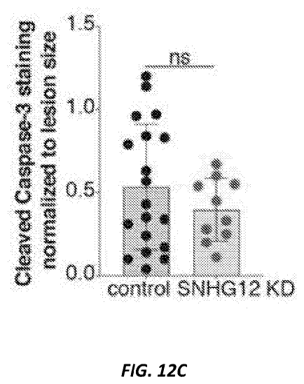

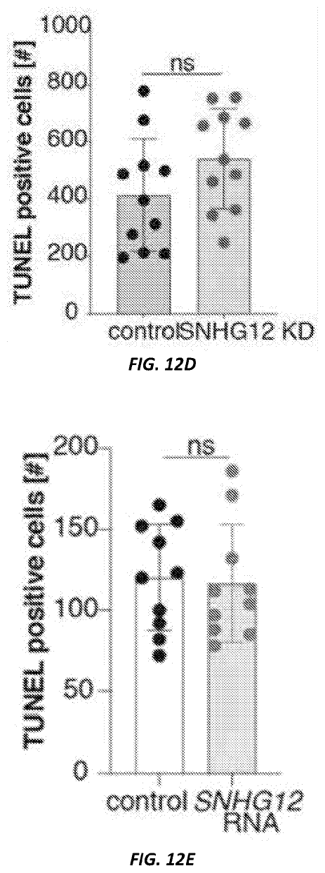

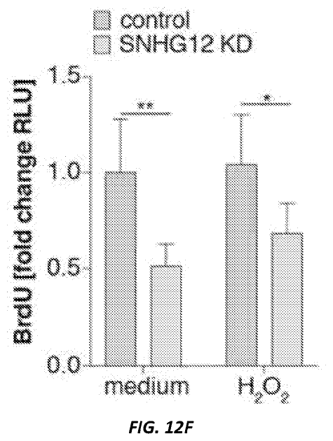

Human primary macrophages were tested for their ability to ingest apoptotic cells, a process described in atherosclerotic plaques as efferocytosis (28, 29). SNHG12-gapmeR transfected macrophages showed a 50% reduced uptake of apoptotic cells (FIG. 11K), whereas lentiviral overexpression of SNHG12 increased their ability to phagocytose apoptotic cells by 2-fold (FIGS. 11L, 11M). Consistently, lesions of SNHG12-gapmeR injected LDLR^{-/-} mice had significantly (P=0.0021) more Mac2-free TUNEL positive cells, an in vivo indication of impaired efferocytosis (FIG. 4K) (29). If DNA damage is unrepaired or extensive, cells may undergo senescence or apoptosis (11). Therefore, markers for apoptosis such as cleaved caspase-3 and TUNEL were analyzed in lesions following SNHG12 knockdown in LDLR^{-/-} mice, but no differences in CD31⁺ and Mac2⁺ were found (FIG. 12A-E). Proliferation, however, was strongly affected upon SNHG12 loss- and gain-of-function experiments in HUVECs (FIGS. 12F, 12G). In summary, these data indicated that SNHG12 deficiency triggers the DDR and DDR-induced senescence, which exacerbates EC permeability, LDL transcytosis, and macrophage efferocytosis.

### Example 5: In Vivo Rescue of DNA Damage by Nicotinamide Riboside (NR) Attenuates SNHG12-Deficient Progression of Atherosclerosis in LDLR^{-/-} Mice

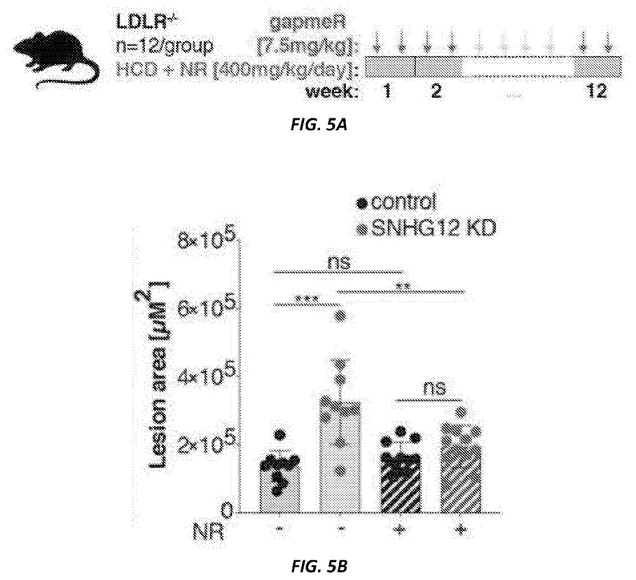

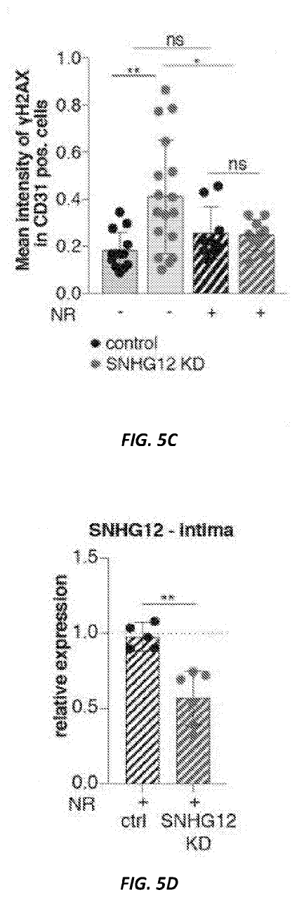

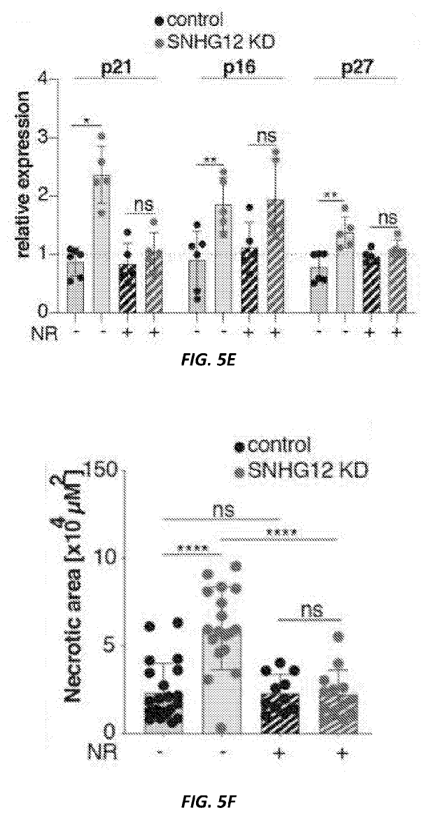

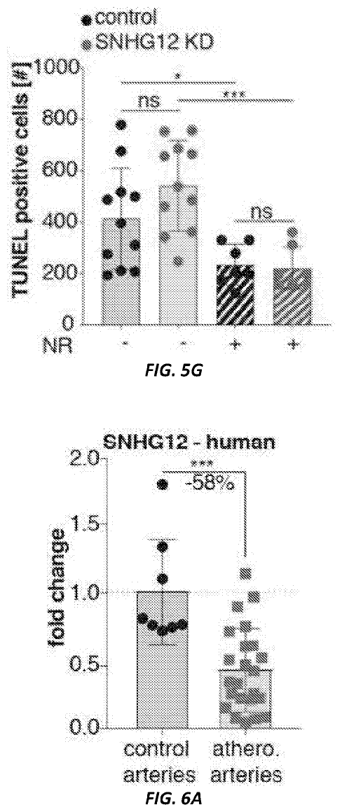

Recent work has demonstrated that ROS-induced DNA damage in tissues relate to impaired NAD⁺ metabolism (30). Furthermore, administration of the NAD⁺ precursor NR can limit DNA damage. Repletion of NAD⁺ by NR also prolonged the life span in mice by rescuing DDR and senescence (30). To assess whether SNHG12-deficient lesion progression involves increased DNA damage, SNHG12-gapmeR was delivered i.v. as described in (FIG. 1F) and the HCD was supplemented with NR (400 mg/kg/day) (FIG. 5A). Analysis of atherosclerotic lesion formation after 12 weeks on HCD and NR showed no significant (P=0.1729) increase in lesion areas by ORO staining between SNHG12-gapmeR and control-gapmeR groups (FIG. 5B). Moreover, NR administration abrogated the SNHG12-gapmeR mediated effect on increased γH2AX foci in lesional ECs in the aortic arch (FIG. 5C). Consistent with less DNA damage, the induction of the senescence markers p16, p21, and p27 fell in the presence of NR compared to LDLR^{-/-} mice treated with SNHG12-gapmeRs without NR (FIGS. 5D, 5E). In addition, NR treatment eliminated differences in plaque necrosis (FIG. 5F), but reduced TUNEL positive cells compared to groups without NR (FIG. 5G). Taken together, the rescue effects mediated by NR strongly support the findings that the lncRNA SNHG12 regulates the DDR both in vitro and in vivo.

### Example 6: Mitochondrial Stress as a Consequence of Accumulating DNA Damage

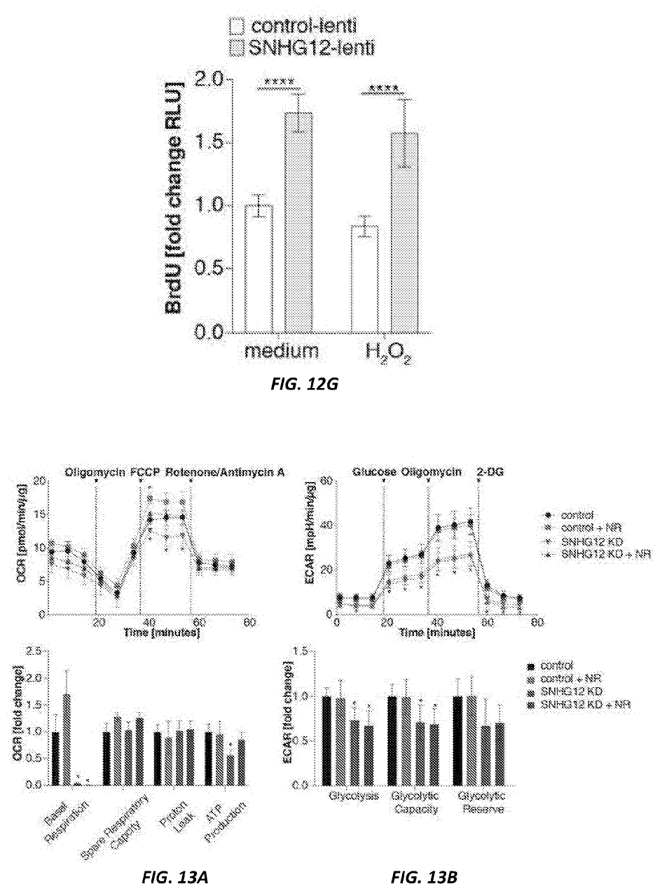

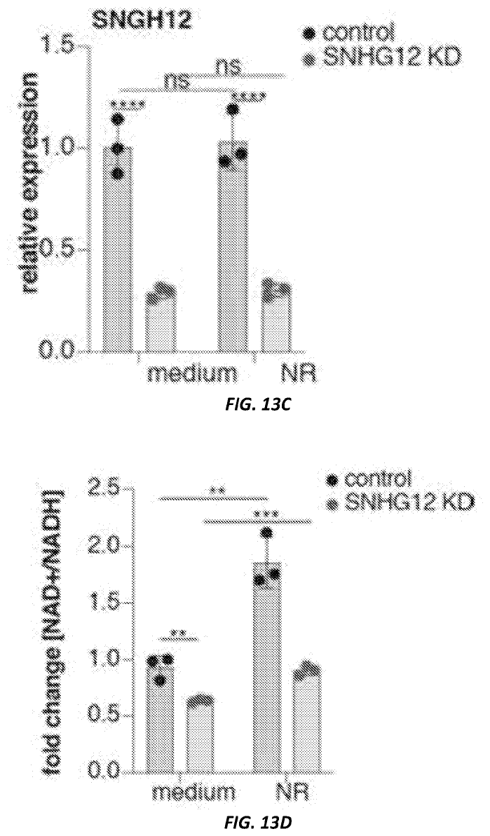

Mitochondrial dysfunction is a hallmark of senescence and reparative responses during oxidative insults requires effective energy metabolism. Oxidative stress-induced DNA damage activates NAD+ consumption pathways (31). Examination of the effect of SNHG12 knockdown on key mitochondrial activities such as mitochondrial respiration and glycolysis in ECs used Seahorse analyses. Loss of SNHG12 decreased the extracellular acidification rate (ECAR) and oxygen consumption rate (OCR) of live cells (FIGS. 13A, 13B). These alteration yielded a reduction in total ATP production (FIG. 13A), likely due to cumulative DNA damage. NR itself did not affect SNHG12 expression (FIG. 13C). However, knockdown of SNHG12 decreased the NAD+/NADH ratio and NR partially rescued this reduction (FIG. 13D). Collectively, these findings support the premise that deficiency of SNHG12 increased DNA damage leading to higher NAD+ consumption, increased mitochondrial stress, and less ATP production.

### Example 7: SNHG12 is a Highly Conserved lncRNA that Inversely Correlates with DNA damage and senescent markers in human, mouse, and pig atherosclerotic specimens

SNHG12 is conserved across mouse, human, and pig (FIG. 1D). To assess the translational relevance of SNHG12, we isolated RNA and protein from human non-diseased control carotid arteries and atherosclerotic carotid arteries. Atherosclerotic arteries (n=23) display significantly reduced expression of SNHG12 (by 58%, P=0.002) compared to control arteries (n=8) (FIG. 6A). Emerging studies demonstrate that DNA damage increases with progression of atherosclerosis (10). Furthermore, in vivo elimination of p16⁺ cells in lesions markedly reduced progression of atherosclerosis, suggesting the importance of senescence for the development of atherosclerosis (13, 32). Consistent with these studies, we found that the expression of γH2AX, p16, and p21 increased markedly (10-fold, 23-fold, and 6.5-fold, respectively) in atherosclerotic carotid arteries compared to control arteries (FIG. 6B). From an independent study, we analyzed specimen samples and RNA-Seq data from Yorkshire pigs that were placed for up to 60 weeks on an HCD and developed atherosclerosis. To this end, carotid cross sections were based on histopathological markers (i.e. ORO, Intima/Media-ratio, CD45, Plaque-internal elastic lamina, elastin) separated into mild, intermediate, and severe groups for progression of atherosclerosis (FIG. 6C). RT-qPCR expression analyses from these groups revealed that SNHG12 also fell ˜50% with progression of disease as previously shown for mouse and human (FIG. 6D). Conversely, the senescence markers p21 and p16 increased in intermediate and severe compared to mild lesions based on RNA-Seq expression data (FIG. 6E). In summary, these results demonstrated not only evolutionary conservation of SNHG12 lncRNA, but its reduced expression with progression of atherosclerosis and a consistent and inverse correlation with DNA damage and senescence across mouse, human, and pig atherosclerotic lesions (FIG. 6F).

### REFERENCES

1. P. Libby, P. M. Ridker, G. K. Hansson, Progress and challenges in translating the biology of atherosclerosis, Nature 473, 317-325 (2011).
2. P. Libby, Inflammation in Atherosclerosis, Arterioscler. Thromb. Vasc. Biol. 32, 2045-2051 (2012).
3. A. Uryga, K. Gray, M. Bennett, DNA Damage and Repair in Vascular Disease, Annu. Rev. Physiol. 78, 45-66 (2016).
4. J. C. Wang, M. Bennett, Aging and atherosclerosis: mechanisms, functional consequences, and potential therapeutics for cellular senescence, Circ. Res. 111, 245-259 (2012).
5. D. Muñoz-Espin, M. Serrano, Cellular senescence: from physiology to pathology, Nature Reviews Molecular Cell Biology 15, 482-496 (2014).
6. F. Violi, R. Carnevale, L. Loffredo, P. Pignatelli, J. I. Gallin, NADPH Oxidase-2 and Atherothrombosis: Insight From Chronic Granulomatous Disease, Arterioscler. Thromb. Vasc. Biol. 37, 218-225 (2017).
7. Y. Oishi, I. Manabe, Macrophages in age-related chronic inflammatory diseases, npj Aging and Mechanisms of Disease 2016 2 2, 16018 (2016).
8. C. Kunsch, R. M. Medford, Oxidative Stress as a Regulator of Gene Expression in the Vasculature, Circ. Res. 85, 753-766 (1999).
9. K. Irani, Oxidant Signaling in Vascular Cell Growth, Death, and Survival: A Review of the Roles of Reactive Oxygen Species in Smooth Muscle and Endothelial Cell Mitogenic and Apoptotic Signaling, Circ. Res. 87, 179-183 (2000).
10. T. Cervelli, A. Borghini, A. Galli, M. G. Andreassi, DNA damage and repair in atherosclerosis: current insights and future perspectives, Int J Mol Sci 13, 16929-16944 (2012).
11. F. D. di Fagagna, P. M. Reaper, L. Clay-Farrace, H. Fiegler, P. Carr, T. von Zglinicki, G. Saretzki, N. P. Carter, S. P. Jackson, A DNA damage checkpoint response in telomere-initiated senescence, Nature 426, 194-198 (2003).
12. T. Minamino, H. Miyauchi, T. Yoshida, Y. Ishida, H. Yoshida, I. Komuro, Endothelial Cell Senescence in Human Atherosclerosis: Role of Telomere in Endothelial Dysfunction, Circulation 105, 1541-1544 (2002).
13. B. G. Childs, D. J. Baker, T. Wijshake, C. A. Conover, J. Campisi, J. M. van Deursen, Senescent intimal foam cells are deleterious at all stages of atherosclerosis, Science (New York, N.Y.) 354, 472-477 (2016).
14. P. Han, C.-P. Chang, Long non-coding RNA and chromatin remodeling, RNA Biol 12, 1094-1098 (2015).
15. I. Ulitsky, A. Shkumatava, C. H. Jan, H. Sive, D. P. Bartel, Conserved Function of lincRNAs in Vertebrate Embryonic Development despite Rapid Sequence Evolution, Cell 147, 1537-1550 (2011).
16. S. Washietl, M. Kellis, M. Garber, Evolutionary dynamics and tissue specificity of human long noncoding RNAs in six mammals, Genome Res. 24, 616-628 (2014).
17. S. Haemmig, M. W. Feinberg, Targeting LncRNAs in Cardiovascular Disease: Options and Expeditions, Circ. Res. 120, 620-623 (2017).
18. T. M. Gottlieb, S. P. Jackson, The DNA-dependent protein kinase: requirement for DNA ends and association with Ku antigen, Cell 72, 131-142 (1993).
19. A. N. Blackford, S. P. Jackson, ATM, ATR, and DNA-PK: The Trinity at the Heart of the DNA Damage Response, Molecular cell 66, 801-817 (2017).
20. J. J. J. Leahy, B. T. Golding, R. J. Griffin, I. R. Hardcastle, C. Richardson, L. Rigoreau, G. C. M. Smith, Identification of a highly potent and selective DNA-dependent protein kinase (DNA-PK) inhibitor (NU7441) by screening of chromenone libraries, Bioorg. Med. Chem. Lett. 14, 6083-6087 (2004).
21. W. G. Nelson, M. B. Kastan, DNA strand breaks: the DNA template alterations that trigger p53-dependent DNA damage response pathways, Molecular and cellular biology 14, 1815-1823 (1994).
22. N. Arnoult, A. Correia, J. Ma, A. Merlo, S. Garcia-Gomez, M. Maric, M. Tognetti, C. W. Benner, S. J. Boulton, A. Saghatelian, J. Karlseder, Regulation of DNA repair pathway choice in S and G2 phases by the NHEJ inhibitor CYREN, Nature 549, 548-552 (2017).
23. J. Stewart-Ornstein, G. Lahav, p53 dynamics in response to DNA damage vary across cell lines and are shaped by efficiency of DNA repair and activity of the kinase ATM, Sci Signal 10, eaah6671 (2017).
24. M. Serrano, A. W. Lin, M. E. McCurrach, D. Beach, S. W. Lowe, Oncogenic ras Provokes Premature Cell Senescence Associated with Accumulation of p53 and p16INK4a, Cell 88, 593-602 (1997).
25. A. Azad, S. Jackson, C. Cullinane, A. Natoli, P. M. Neilsen, D. F. Callen, S.-M. Maira, W. Hackl, G. A. McArthur, B. Solomon, Inhibition of DNA-Dependent Protein Kinase Induces Accelerated Senescence in Irradiated Human Cancer Cells, Mol Cancer Res 9, 1696-1707 (2011).
26. S. M. Armstrong, M. G. Sugiyama, K. Y. Y. Fung, Y. Gao, C. Wang, A. S. Levy, P. Azizi, M. Roufaiel, S.-N. Zhu, D. Neculai, C. Yin, S.-S. Bolz, N. G. Seidah, M. I. Cybulsky, B. Heit, W. L. Lee, A novel assay uncovers an unexpected role for SR-BI in LDL transcytosis, Cardiovasc. Res. 108, 268-277 (2015).
27. G. Franck, T. Mawson, G. Sausen, M. Salinas, G. S. Masson, A. Cole, M. Beltrami-Moreira, Y. Chatzizisis, T. Quillard, Y. Tesmenitsky, E. Shvartz, G. K. Sukhova, F. K. Swirski, M. Nahrendorf, E. Aikawa, K. J. Croce, P. Libby, Flow Perturbation Mediates Neutrophil Recruitment and Potentiates Endothelial Injury via TLR2 in Mice: Implications for Superficial Erosion, Circ. Res. 121, 31-42 (2017).
28. X. Liao, J. C. Sluimer, Y. Wang, M. Subramanian, K. Brown, J. S. Pattison, J. Robbins, J. Martinez, I. Tabas, Macrophage Autophagy Plays a Protective Role in Advanced Atherosclerosis, Cell Metab. 15, 545-553 (2012).
29. Y. Wang, M. Subramanian, A. Yurdagul Jr., V. C. Barbosa-Lorenzi, B. Cai, J. de Juan-Sanz, T. A. Ryan, M. Nomura, F. R. Maxfield, I. Tabas, Mitochondrial Fission Promotes the Continued Clearance of Apoptotic Cells by Macrophages, Cell 171, 331-345.e22 (2017).
30. H. Zhang, D. Ryu, Y. Wu, K. Gariani, X. Wang, P. Luan, D. D'Amico, E. R. Ropelle, M. P. Lutolf, R. Aebersold, K. Schoonjans, K. J. Menzies, J. Auwerx, NAD repletion improves mitochondrial and stem cell function and enhances life span in mice, Science (New York, N.Y.) 352, 1436-1443 (2016).
31. H. Massudi, R. Grant, N. Braidy, J. Guest, B. Farnsworth, G. J. Guillemin, M. Polymenis, Ed. Age-Associated Changes In Oxidative Stress and NAD+ Metabolism In Human Tissue, PLoS ONE 7, e42357 (2012).
32. P. Libby, Assisted Living in the Atheroma: Elderly Macrophages Promote Plaques, Cell Metab. 24, 779-781 (2016).
33. L. J. Kienker, E. K. Shin, K. M. N. A. research, 2000, Both V(D)J recombination and radioresistance require DNA-PK kinase activity, though minimal levels suffice for V(D)J recombination. Nucleic Acids Research 28, 2752-2761 (2000).
34. W. Martinet, M. W. M. Knaapen, G. R. Y. De Meyer, A. G. Herman, M. M. Kockx, Elevated levels of oxidative DNA damage and DNA repair enzymes in human atherosclerotic plaques, Circulation 106, 927-932 (2002).
35. N. Botto, A. Rizza, M. G. Colombo, A. M. Mazzone, S. Manfredi, S. Masetti, A. Clerico, A. Biagini, M. G. Andreassi, Evidence for DNA damage in patients with coronary artery disease, Mutation Research/Genetic Toxicology and Environmental Mutagenesis 493, 23-30 (2001).
36. W. Martinet, M. W. Knaapen, G. R. De Meyer, A. G. Herman, M. M. Kockx, Oxidative DNA damage and repair in experimental atherosclerosis are reversed by dietary lipid lowering, Circ. Res. 88, 733-739 (2001).
37. J. G. Schneider, B. N. Finck, J. Ren, K. N. Standley, M. Takagi, K. H. Maclean, C. Bernal-Mizrachi, A. J. Muslin, M. B. Kastan, C. F. Semenkovich, ATM-dependent suppression of stress signaling reduces vascular disease in metabolic syndrome, Cell Metab. 4, 377-389 (2006).
38. D. Hanahan, R. A. Weinberg, Hallmarks of Cancer: The Next Generation, Cell 144, 646-674 (2011).
39. J. Song, X. Wu, R. Ma, L. Miao, L. Xiong, W. Zhao, Long noncoding RNA SNHG12 promotes cell proliferation and activates Wnt/β-catenin signaling in prostate cancer through sponging microRNA-195, Journal of cellular biochemistry 120, 13066-13075 (2019).
40. B. F. Yang, W. Cai, B. C. E. R. M. P. Sci, 2018, LncRNA SNHG12 regulated the proliferation of gastric carcinoma cell BGC-823 by targeting microRNA-199a/b-5p, Eur Rev Med Pharmacol Sci 22, 1297-1306 (2018).
41. O. Wang, F. Yang, Y. Liu, L. Lv, R. Ma, C. Chen, J. Wang, Q. Tan, Y. Cheng, E. Xia, Y. Chen, X. Zhang, C-MYC-induced upregulation of lncRNA SNHG12 regulates cell proliferation, apoptosis and migration in triple-negative breast cancer, American Journal of Translational Research 9, 533-545 (2017).
42. Z.-B. Liu, C. Tang, X. Jin, S.-H. Liu, W. Pi, Increased expression of lncRNA SNHG12 predicts a poor prognosis of nasopharyngeal carcinoma and regulates cell proliferation and metastasis by modulating Notch signal pathway, Cancer Biomarkers 23, 603-613 (2018).
43. K. Gray, S. Kumar, N. Figg, J. Harrison, L. Baker, J. Mercer, T. Littlewood, M. Bennett, Effects of DNA damage in smooth muscle cells in atherosclerosis, Circ. Res. 116, 816-826 (2015).
44. J. D. Lambeth, NOX enzymes and the biology of reactive oxygen, Nat. Rev. Immunol. 4, 181-189 (2004).
45. R. S. Balaban, S. Nemoto, T. Finkel, Mitochondria, Oxidants, and Aging, Cell 120, 483-495 (2005).
46. C. Cantó, R. H. Houtkooper, E. Pirinen, D. Y. Youn, M. H. Oosterveer, Y. Cen, P. J. Fernandez-Marcos, H. Yamamoto, P. A. Andreux, P. Cettour-Rose, K. Gademann, C. Rinsch, K. Schoonjans, A. A. Sauve, J. Auwerx, The NAD+ Precursor Nicotinamide Riboside Enhances Oxidative Metabolism and Protects against High-Fat Diet-Induced Obesity, Cell Metab. 15, 838-847 (2012).
47. P. Tontonoz, X. Wu, M. Jones, Z. Zhang, D. Salisbury, T. Sallam, Long Noncoding RNA Facilitated Gene Therapy Reduces Atherosclerosis in a Murine Model of Familial Hypercholesterolemia, Circulation 136, 776-778 (2017).
48. T. Sallam, M. Jones, B. J. Thomas, X. Wu, T. Gilliland, K. Qian, A. Eskin, D. Casero, Z. Zhang, J. Sandhu, D. Salisbury, P. Rajbhandari, M. Civelek, C. Hong, A. Ito, X. Liu, B. Daniel, A. J. Lusis, J. Whitelegge, L. Nagy, A. Castrillo, S. Smale, P. Tontonoz, Transcriptional regulation of macrophage cholesterol efflux and atherogenesis by a long noncoding RNA, Nat Med 24, 304-312 (2018).
49. S. Cremer, K. M. Michalik, A. Fischer, L. Pfisterer, N. Jae, C. Winter, R. A. Boon, M. Muhly-Reinholz, D. John, S. Uchida, C. Weber, W. Poller, S. Gunther, T. Braun, D. Y. Li, L. Maegdefessel, L. Perisic Matic, U. Hedin, O. Soehnlein, A. Zeiher, S. Dimmeler, Hematopoietic Deficiency of the Long Noncoding RNA MALAT1 Promotes Atherosclerosis and Plaque Inflammation, Circulation 139, 1320-1334 (2019).
50. E. Thorp, G. Li, T. A. Seimon, G. Kuriakose, D. Ron, I. Tabas, Reduced apoptosis and plaque necrosis in advanced atherosclerotic lesions of Apoe−/− and Ldlr−/− mice lacking CHOP, Cell Metab. 9, 474-481 (2009).
51. D. Won, S.-N. Zhu, M. Chen, A.-M. Teichert, J. E. Fish, C. C. Matouk, M. Bonert, M. Ojha, P. A. Marsden, M. I. Cybulsky, Relative Reduction of Endothelial Nitric-Oxide Synthase Expression and Transcription in Atherosclerosis-Prone Regions of the Mouse Aorta and in an in Vitro Model of Disturbed Flow, The American journal of pathology 171, 1691-1704 (2007).
52. P. F. Davies, M. Civelek, Y. F. Cardiovascular, 2013, The atherosusceptible endothelium: endothelial phenotypes in complex haemodynamic shear stress regions in vivo, Cardiovasc Res 99, 315-327 (2013).
53. Z. Cao, A. K. Wara, B. Icli, X. Sun, R. R. S. Packard, F. Esen, C. J. Stapleton, M. Subramaniam, K. Kretschmer, I. Apostolou, H. von Boehmer, G. K. Hansson, T. C. Spelsberg, P. Libby, M. W. Feinberg, Kruppel-like factor KLF10 targets transforming growth factor-beta1 to regulate CD4(+)CD25(−) T cells and T regulatory cells, J. Biol. Chem. 284, 24914-24924 (2009).
54. X. Sun, S. He, A. K. M. Wara, B. Icli, E. Shvartz, Y. Tesmenitsky, N. Belkin, D. Li, T. S. Blackwell, G. K. Sukhova, K. Croce, M. W. Feinberg, Systemic delivery of microRNA-181b inhibits nuclear factor-KB activation, vascular inflammation, and atherosclerosis in apolipoprotein E-deficient mice, Circ. Res. 114, 32-40 (2014).
55. X. Sun, B. Icli, A. K. Wara, N. Belkin, S. He, L. Kobzik, G. M. Hunninghake, M. P. Vera, MICU Registry, T. S. Blackwell, R. M. Baron, M. W. Feinberg, MicroRNA-181b regulates NF-κB-mediated vascular inflammation, J. Clin. Invest. 122, 1973-1990 (2012).
56. S. Ghaffari, F. N. Nabi, M. G. Sugiyama, W. L. Lee, Estrogen Inhibits LDL (Low-Density Lipoprotein) Transcytosis by Human Coronary Artery Endothelial Cells via GPER (G-Protein-Coupled Estrogen Receptor) and SR-BI (Scavenger Receptor Class B Type 1), Arterioscler. Thromb. Vasc. Biol. 38, 2276-2277 (2018).
57. S. B. Ficarro, Y. Zhang, Y. Lu, A. R. Moghimi, M. Askenazi, E. Hyatt, E. D. Smith, L. Boyer, T. M. Schlaeger, C. J. Luckey, J. A. Marto, Improved electrospray ionization efficiency compensates for diminished chromatographic resolution and enables proteomics analysis of tyrosine signaling in embryonic stem cells, Anal. Chem. 81, 3440-3447 (2009).
58. K. C. Koskinas, Y. S. Chatzizisis, M. I. Papafaklis, A. U. Coskun, A. B. Baker, P. Jarolim, A. Antoniadis, E. R. Edelman, P. H. Stone, C. L. Feldman, Synergistic effect of local endothelial shear stress and systemic hypercholesterolemia on coronary atherosclerotic plaque progression and composition in pigs, Int. J. Cardiol. 169, 394-401 (2013).
59. L. Wang, H. J. Park, S. Dasari, S. Wang, J.-P. Kocher, W. Li, CPAT: Coding-Potential Assessment Tool using an alignment-free logistic regression model, Nucleic Acids Res. 41, e74-e74 (2013).
60. A. Dobin, C. A. Davis, F. Schlesinger, J. Drenkow, C. Zaleski, S. Jha, P. Batut, M. Chaisson, T. R. Gingeras, STAR: ultrafast universal RNA-seq aligner, Bioinformatics 29, 15-21 (2013).
61. F. Garcia-Alcalde, K. Okonechnikov, J. Carbonell, L. M. Cruz, S. Gotz, S. Tarazona, J. Dopazo, T. F. Meyer, A. Conesa, Qualimap: evaluating next-generation sequencing alignment data, Bioinformatics 28, 2678-2679 (2012).
62. Y. Liao, G. K. Smyth, W. Shi, featureCounts: an efficient general purpose program for assigning sequence reads to genomic features, Bioinformatics 30, 923-930 (2014).
63. M. I. Love, W. Huber, S. Anders, Moderated estimation of fold change and dispersion for RNA-seq data with DESeq2, Genome Biology 15, 550 (2014).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject who has a disease associated with a malfunction in DNA repair response, the method comprising: administering to the subject a therapeutically effective dose of a pharmaceutical composition that increases expression of Small Nucleolar Host Gene-12 (SNHG12) long non-coding RNA in a cell of the subject in need thereof.

2. The method of claim 1, wherein the pharmaceutical composition comprises a nucleic acid molecule comprising (i) all or part of the SNHG12 long-coding RNA sequence, or (ii) a sequence, optionally in an expression vector, encoding all or part of the SNHG12 long-coding RNA.

3. The method of claim 3, wherein the expression vector comprises an adeno-associated virus (AAV), adenovirus, lentivirus, or a DNA plasmid.

4. The method of claim 1, wherein the nucleic acid molecule is an RNA molecule comprising all or part of SEQ ID NO: 1 or 2.

5. The method of claim 1, wherein the nucleic acid molecule comprises SEQ ID NO: 1 or 2.

6. The method of claim 4, wherein the nucleic acid molecule has at least 90% of sequence identity with SEQ ID NO: 1 or 2.

7. The method of claim 1, wherein the composition is administered to the subject parenterally, intramuscularly, intravitreally, subcutaneously, arterially, intravenously, topically, orally, or by local administration, such as by aerosol or transdermally.

8. The method of claim 2, wherein the nucleic acid molecule comprises a chemical modification that improves one or more, or all, of nuclease stability, decreased likelihood of triggering an innate immune response, lowering incidence of off-target effects, and improved pharmacodynamics relative to a non-modified nucleic acid.

9. The method of claim 9, wherein the at least one chemical modification comprises a modification selected from phosphorothioate, boranophosphate, 4′-thio-ribose, locked nucleic acid, 2′-O-(2′-methoxyethyl), 2′-O-alkyl, 2′-O-alkyl-O-alkyl, 2′-O-methyl, 2′-fluoro, 2′-amino, or 2′-deoxy-2′-fluoro-b-D-arabinonucleic acid.

10. The method of claim 9, wherein the at least one chemical modification comprises a 5′ cap.

11. The method of claim 1, wherein the composition is administered to the tunica intima of the subject.

12. The method of claim 1, wherein the disease is atherosclerosis, heart failure, diabetes, hypertension, neurodegenerative disease, autoimmune disease, ataxia telangiectasia, aging, Bloom's Syndrome, immunodeficiency, Cockayne syndrome, Nijmegen breakage syndrome, Trichothiodystrophy, Fanconi Anaemia, Werner Syndrome, Li-Fraumeni syndrome, xeroderma pigmentosum, senescence, Hutchinson-Gilford progeria syndrome, or cancer.

13. A pharmaceutical composition for use in treating a subject suffering from a disease associated with a malfunction in DNA repair response, comprising a nucleic acid molecule comprising (i) all or part of the SNHG12 long-coding RNA sequence, or (ii) a sequence encoding all or part of the SNHG12 long-coding RNA, optionally in an expression vector.

14. The pharmaceutical composition for the use of claim 14, wherein the expression vector comprises an adeno-associated virus (AAV), adenovirus, lentivirus, or a DNA plasmid.

15. The pharmaceutical composition for the use of claim 14, wherein the nucleic acid comprises all or part of SEQ ID NO: 1 or 2.

16. The pharmaceutical composition for the use of claim 16, wherein the nucleic acid has at least 80% sequence identity with SEQ ID NO: 1 or 2 and is capable of increasing the expression of Small Nucleolar Host Gene-12 (SNHG12) long non-coding RNA.

17. The pharmaceutical composition for the use of claim 16, wherein the nucleic acid is SEQ ID NO: 1 or 2.

18. The pharmaceutical composition for the use of claim 16, wherein the nucleic acid has at least 90% sequence identity with SEQ ID NO: 1 or 2.

19. The pharmaceutical composition for the use of claim 14, wherein the nucleic acid comprises a chemical modification that improves one or more or all of nuclease stability, decreased likelihood of triggering an innate immune response, lowering incidence of off-target effects, and improved pharmacodynamics relative to a non-modified nucleic acid.

20-21. (canceled)

22. The pharmaceutical composition for the use of claim 14, wherein the at least one chemical modification is selected from phosphorothioate, boranophosphate, 4′-thio-ribose, locked nucleic acid, 2′-O-(2′-methoxyethyl), 2′-O-alkyl, 2′-O-alkyl-O-alkyl, 2′-O-methyl, 2′-fluoro, 2′-amino, or 2′-deoxy-2′-fluoro-b-D-arabinonucleic acid.
